SEQUENCE LISTING (4 sequences)

SEQ ID NO: 1 - RNA, 21 nt, synthetic construct
  source 1..21: mol_type = other RNA
taaagtgctgacagtgcagat

SEQ ID NO: 2 - RNA, 23 nt, synthetic construct
  source 1..23: mol_type = other RNA
ttattgcttaagaatacgcgtag

SEQ ID NO: 3 - skipped

SEQ ID NO: 4 - protein, 5 aa, synthetic construct
FLFLF

(19) United States
(12) Patent Application Publication
(10) Pub. No.: US 2023/0248751 A1
(43) Pub. Date: Aug. 10, 2023

(54) NOVEL BIOENERGETIC/ANTI INFLAMMATORY THERAPY

(71) Applicant: Nour Heart, Inc., Vienna, VA (US)

(72) Inventor: Salwa A. Elgebaly, Vienna, VA (US)

(21) Appl. No.: 18/190,814

(22) Filed: Mar. 27, 2023

Related U.S. Application Data
(63) Continuation-in-part of application No. 16/948,235, filed on Sep. 9, 2020, now Pat. No. 11,654,134.
Application No. 16/948,235 is a continuation-in-part of application No. 16/719,723, filed on Dec. 18, 2019, now Pat. No. 10,859,573.
Application No. 16/719,723 is a continuation-in-part of application No. 16/252,402, filed on Jan. 18, 2019, now Pat. No. 10,895,572.
(60) Provisional application No. 62/686,184, filed on Jun. 18, 2018.
(60) Provisional application No. 62/686,184, filed on Jun. 18, 2018.
(60) Provisional application No. 63/002,179, filed on Mar. 30, 2020.

(51) Int. Cl. A61K 31/675; A61B 5/00; A61K 31/4168; A61P 9/10

(52) U.S. Cl. CPC A61K 31/675; A61B 5/4848; A61K 31/4168; A61P 9/10

(57) ABSTRACT

The present invention provides a novel mechanism based on a new paradigm involving up-regulation of Nourin protein and its downstream regulatory network discovered in cardiac systems for its high energy demand and targeting such ischemic events and ischemia-induced disease in another high energy and ATP requiring organ system, that of brain and neuro system and discloses a method for prevention and treatment of neuro diseases and neurological surgical procedures using the bioenergetic drugs, Cyclocreatine (CCr) and Cyclocreatine Phosphate (CCrP). Bioenergetic drugs of the present invention are used for their targeting of depletion of ATP, anti-inflammatory, and anti-apoptotic functions against downstream ischemia-induced injury and tissue deterioration events including, inflammation, apoptosis, necrosis, organ failure, and loss of organ function. The presently disclosed mechanism and methods of prevention and treatment work as potent cardioprotective drugs by preserving cellular energy source, preventing ischemic injury, rejuvenating organ function, thus, maintaining normal physical activity. The present invention provides CCr and CCrP as a new preventative and therapeutic approach to prevent and treat disease development and progression in subjects with neuro ischemic diseases and disorders involving ischemia-induced injury, including aging-related disorders, such as brain ischemia (stroke), Alzheimer’s disease, ocular ischemia-induced injury and diseases, optic nerve ischemia-induced injury and diseases, and retinal ischemia-induced injury and diseases, as well as for neurologic surgical procedures. Thus, the present disclosure provides a Nourin targeting therapy for both cardiovascular and neuro systems where with CCrP’s anti-inflammatory and anti-apoptotic activities, it will prevent ischemic injury restoring organ function without affecting the host defense immunity without subjecting the subjects to immunosuppression as seen with traditional therapies such as steroid treatments.

### CROSS REFERENCE TO RELATED APPLICATIONS

The current application is a Patent Cooperation Treaty (PCT) application and claims benefit of U.S. Pat. Application 16/948,235 filed Sep. 9, 2020. The U.S. Pat. Application 16/948,235 is a continuation-in-part of the U.S. Pat. Application 16/252,402 filed Jan. 18, 2019 which claims benefit of the U.S. Provisional Pat. Application 62/686,184 filed Jun. 18, 2018. The current application is a continuation-in-part of the U.S. Pat. Application 16/719,723 filed Dec. 18, 2019 which is a continuation-in-part of the U.S. Pat. Application 16/252,402 filed Jan. 18, 2019, wherein U.S. Pat. Application 16/252,402 claims benefit of U.S. Provisional Pat. Application 62/686,184 filed Jun. 18, 2018. The current application claims benefit of U.S. Provisional Pat. Application 63/002,179 filed Mar. 30, 2020.

### SEQUENCE LISTING

The instant application contains a Sequence Listing which has been submitted electronically in ASCII (XML) format and is hereby incorporated by reference in its entirety. Said ASCII copy, created on Mar. 15, 2023, is named TUP72779_Seq listing.XML and is 3,897 bytes in size generated in ST26 format.

### FIELD OF THE INVENTION

The present invention generally relates to the fields of medicine, physiology, biochemistry, molecular, prevention, medical treatments, procedures and surgeries. The present invention particularly relates to the use of the novel bioenergetic drug, Cyclocreatine Phosphate (CCrP) as a potent cardioprotective drug by preserving cellular ATP energy source, preventing ischemic injury, rejuvenating organ function, thus, maintaining normal physical activity. Cyclocreatine Phosphate can be used as a new therapeutic approach to prevent disease development and progression, and to treat ischemia-induced, as well as aging-related cardiovascular and neurodegenerative diseases. More specifically, the present invention provides methods to prevent and treat patients with ischemia-induced and aging-related diseases, including stroke. Alzheimer’s disease, ocular ischemia-induced injury and diseases, optic nerve ischemia-induced injury and diseases, and retinal ischemia-induced injury and diseases as well as methods to prevent and treat ischemic events and ischemia-induced disease associated with neurological surgical procedures.

### BACKGROUND OF THE INVENTION

Heart failure (HF) is a clinical diagnosis when the heart fails to provide sufficient circulatory force to meet the body’s metabolic requirements. It is one of the major causes of mortality in the United States, responsible for ~30% of patient deaths annually. HF is the final manifestation of cardiovascular disease (CVD) and cardiac injury. The lifetime risk for HF is substantial. It is strongly age dependent, with incidence rates of <1% below the age of 50 years and up to 30% at advanced ages (>80 years). According to the American Heart Association’s Heart and Stroke Facts, the prevalence of HF will increase ≈50% between 2012 and 2030. resulting in >8 million people at ≥18 years of age with HF. This daunting future reflects the increased prevalence of HF as the population ages, AMI survival improves, and HF survival itself increases at rates that exceed the scientific and medical impact to prevent the development of HF. HF is becoming an increasing concern to healthcare worldwide because of the increasing disease burden and economic impact. It is the only cardiovascular disorder that continues to increase in both prevalence and incidence, and as the population continues to age, it is expected that the prevalence of this disease will continue to rise. Admissions for acute heart failure continue to increase but, to date, no new therapies have improved clinical outcomes.

HF has primarily been recognized as a disease of the elderly population (>60 years) and is reported to affect about 2% to 3% of people in the United States. Of these include 10% of males and 8% of females. Unfortunately, these numbers are on a gradual increase due to the on-going prevalence of HF with increasing age. In the United States itself, about more than three million physician visits per year have been accounted for patients with HF as the primary health issue. In 2013, the total number of HF patients were 5.1 million, and direct costs were equal to $32 billion; and this cost is being projected to increase by about three-fold by 2030. As of 2011, the estimated lifetime cost of HF per individual patient was $110,000/year, with more than three-fourths of this cost consumed by ‘in-hospital care’. It is predicted that as the population ages, the direct medical costs of all cardiovascular diseases (including hypertension, coronary heart disease, stroke, and heart failure) will triple, reaching $818 billion in 2030.

Natriuretic peptide (NP; B-type NP (BNP), N-terminal proBNP (NT-proBNP), and mid regional proANP (MR-proANP)] concentrations are the known quantitative plasma biomarkers for the presence and severity of hemodynamic cardiac stress and HF. NPs are used in conjunction with all other clinical information and they are surrogates for intracardiac volumes and filling pressures. NPs are measured in patients presenting with symptoms suggestive of HF such as dyspnea (difficulty breathing) and/or fatigue, as their use facilitates the early diagnosis and risk stratification of HF and they have very high diagnostic accuracy in discriminating HF from other causes of dyspnea: the higher the NP, the higher the likelihood that dyspnea is caused by HF.

However, limitations of Natriuretic peptides, include:
1) The grey zone levels: BNP is indicative of only stress and muscle stretch, but not cell injury, thus, it needs to be combined with concomitant clinical features: such as a history of HF, jugular venous pressure and prior diuretic use.
2) Patients with acute and chronic ischemia: Natriuretic peptides do not seem to provide added diagnostic information on top of clinical judgement and/or Troponin measurements in the detection of inducible myocardial ischemia.
3) Renal impairment: it affects NPs level.

Thus, there is still an unmet clinical need related to HF patients since physicians’ face challenges in identification and treatment of patients with myocardial ischemia who may be exposed to a higher risk of adverse outcomes such as re-infarction, early cardiac dysfunction, HF, and death. The current approaches for stratifying the risk of cardiac dysfunction in patients with AMI is based on clinical judgment, echocardiographic findings, and measurement of some selected biomarkers, namely cardio-specific Troponins and natriuretic peptides. Diagnostic imaging of the heart by means of echocardiography or magnetic resonance imaging (MRI) is commonplace, but only allows a physician to capture a sporadic and virtually instantaneous picture of cardiac function that, without serial monitoring, does not provide an absolute perception of the future risk for developing HF.

Heart and brain are among parts of the body requiring the greatest amounts of energy and they are the most affected during failures of the mitochondria to generate sufficient cellular adenosine triphosphate (ATP) during hypoxia and ischemia. Both organs are sensitive to changes in oxygen supply and ischemia, and inflammation play a key role in acute stroke and acute myocardial infarction. Similarly, spinal cord ischemia is associated with a loss of neuromuscular function, and retinal and optic nerve ischemia results in vision loss. ATP depletion in ischemic heart and similarly in the brain/spinal cord, is a critical contributor to the pathogenic event that triggers cell injury, inflammation/fibrosis, apoptosis, leading to organ dysfunction. With aging, a decline in mitochondrial function and a reduction of oxygen supply associated with vascular dysfunction, increases the incidence of heart failure (HF), stroke, and Alzheimer Disease (AD). Although the cause of AD remains uncertain until now, it is defined pathologically by mitochondrial dysfunction and loss of ATP, as well as loss of synaptic and neuronal function due to the downstream accumulation of amyloid-beta peptide (Aβ) plaques and tau protein triggered by oxidative stress and inflammation. Additionally, while no effective treatment exists, current therapy provides symptomatic relief without being able to prevent, stop, or reverse the pathologic process of AD. Currently, no effective treatment exists for AD patients, but available therapies provide symptomatic relief without being able to prevent, stop, slow, or reverse the disease. No prior studies have addressed the problem of inefficient production of ATP within the neural tissue by directly increasing ATP supply via the phosphocreatine system. Nourin is a 3 KDa N-formyl peptide and the formylated peptide Nourin is a potent inflammatory mediator which stimulates leukocyte chemotaxis, adhesion and activation to release a number of cytokine and chemokine mediators, adhesion molecules, digestive enzymes and free radicals. In vivo, the injection of human cardiac Nourin into rabbit skin resulted in an acute inflammatory response within the first 30 minutes characterized by a significant neutrophil infiltration. Nourin can, thus, be characterized as an Alarmin that promotes the innate immune response since it is rapidly released by local myocardial tissues following ischemia and contributes to the initiation and amplification of post-reperfusion myocardial inflammation. As such, Nourin can be an important diagnostic and therapeutic target. Nourin works as a ligand on leukocyte formyl peptide receptors (FPR) that are important potential therapeutic targets to control early and late post-reperfusion inflammation and injury.

Nourin can identify patients at increased risk of developing HF due to ischemia, and, therefore, will permit early crucial therapeutic strategy to alleviate ischemic injury. Targeted therapy would include revascularization (e.g., bypass surgery) and/or medical treatment such as the bioenergetic drugs, 1-carboxymethyl-2-iminoimidazolidine referred to as Cyclocreatine (CCr) and l-carboxymethyl-2-imino-3-phosphonoimidazolidine referred to as Cyclocreatine Phosphate (CCrP).

Wozincki D. T., et al., has previously disclosed that the ability of 1-carboxymethyl-2-imino-3-phosphonoimidazolidine (cyclocreatine-P), accumulated by a simple brain model, to function as a supplemental synthetic phosphagen and respond to the decreases in cytosolic ATP/free ADP ratios that occur during prolonged stimulation by various excitatory amino acids was investigated. Suspensions of chopped whole brain from 11- to 14-day-old chick embryos were incubated with 30 mM cyclocreatine for 90 min, resulting in accumulation of 100 mumol/g dry weight of cyclocreatine-P, and then incubated for up to 1 h with a series of excitatory amino acids of widely differing potencies. Under these conditions net utilization of cyclocreatine-P was detected in response to stimulation by the following neuroexcitatory compounds at the indicated threshold concentrations: 4ainite (20 microM), N-methyl-DL-aspartate (20 microM), L-homocysteate (20 microM), L-glutamate (200 microM), D-glutamate (200 microM), L-aspartate (2 mM), DL-2-amino-3-phosphonopropionate (2 mM), and DL-2-amino-4-phosphonobutyrate (2 mM). Significant increases in water content of chick embryo brain minces accompanied stimulation by excitatory amino acids. It is suggested that changes in water content or cyclocreatine-P levels in this sensitive brain model might be utilized in automatable screening procedures for detecting novel antagonists and/or new agonists of excitatory amino acids.

Matthews R.T., et al., has previously disclosed that the gene defect in Huntington’s disease (HD) may result in an impairment of energy metabolism. Malonate and 3-nitropropionic acid (3-NP) are inhibitors of succinate dehydrogenase that produce energy depletion and lesions that closely resemble those of HD. Oral supplementation with creatine or cyclocreatine, which are substrates for the enzyme creatine kinase, may increase phosphocreatine (PCr) or phosphocyclocreatine (PCCr) levels and ATP generation and thereby may exert neuroprotective effects. It was found that oral supplementation with either creatine or cyclocreatine produced significant protection against malonate lesions, and that creatine but not cyclocreatine supplementation significantly protected against 3-NP neurotoxicity. Creatine and cyclocreatine increased brain concentrations of PCr and PCCr, respectively, and creatine protected against depletions of PCr and ATP produced by 3-NP. Creatine supplementation protected against 3-NP induced increases in striatal lactate concentrations in vivo as assessed by 1H magnetic resonance spectroscopy. Creatine and cyclocreatine protected against malonate-induced increases in the conversion of salicylate to 2,3- and 2,5-dihydroxybenzoic acid, biochemical markers of hydroxyl radical generation. Creatine administration protected against 3-NP-induced increases in 3-nitrotyrosine concentrations, a marker of peroxynitrite-mediated oxidative injury. Oral supplementation with creatine or cyclocreatine results in neuroprotective effects in vivo, which may represent a novel therapeutic strategy for HD and other neurodegenerative diseases. Roberts, J.J. et al., has previously disclosed that a new creatine analog, 1-carboxyethyl-2-iminoimidazolidine (homocyclocreatine), has been synthesized and compared with other synthetic analogs of creatine as a substrate for creatine kinase under both in vitro and in vivo conditions. Reactivity with rabbit muscle creatine kinase at 2 mM and pH 7.0 occurred in the order: creatine greater than cyclocreatine (1-carboxymethyl-2-iminoimidazolidine) greater than N-ethylguanidinoacetate greater than N-propylguanidinoacetate greater than guanidinoacetate greater than N-methyl-3-guanidinopropionate greater than 3-guanidinopropionate greater than homocyclocreatine. Homocyclocreatine was 10,000-fold less active than creatine. In the reverse direction at 0.2 mM and pH 7.0: creatine-P greater than N-ethylguanidinoacetate-P greater than cyclocreatine-P much greater than homocyclocreatine-P. Homocyclocreatine-P was 200,000-fold less active than creatine-P. The phosphoryl group transfer potential of homocyclocreatine-P was estimated to be 2 kcal/mol lower than that of creatine-P. Chicks fed 5% homocyclocreatine for 16 days synthesized and accumulated homocyclocreatine-P in breast muscle (32 µmol/g wet wt), leg muscle (24 µmol/g), heart (7 µmol/g), intestine (8.5 µmol/g), and brain (2.4 µmol/g). During ischemia homocyclocreatine-P was utilized by muscle much more slowly for the regeneration of ATP than was creatine-P or cyclocreatine-P. Our results suggest that in tissues of homocyclocreatine-fed animals subjected to a sudden large increase in work load or to ischemia, the residual creatine-P system would rapidly equilibrate with the adenylate system at the new lower cytosolic phosphorylation potential, whereas in the same cytosol the (homocyclocreatine-P)/(homocyclocreatine) ratio would exhibit a hysteresis or memory effect and reflect for a considerable period of time the earlier higher (ATP)/(free ADP) ratio rather than the actual lower (ATP)/(free ADP) ratio.

To address the above-discussed need for effective preventative and therapeutic treatments against neuro diseases involving aging and/or ischemic injury such as Alzheimer’s disease, stroke, spinal cord, and ocular, etc., and given that the neuro system (brain, spinal cord, and ocular retina/optic nerve) is very similar to the cardiovascular system (heart and vessels), the present disclosure investigates and provides, based on the heart studies and results on protection, prevention and treatment against ischemia injury in cardiac systems using CCr and CCrP and provide a similar Nourin targeting therapy for neuro system.

### SUMMARY OF THE INVENTION

In an ischemia-induced and often times, aging-related disorder of the heart, that of heart failure (HP), the bioenergetic drug of the present disclosure, 1-carboxymethyl-2-imino-3-phosphonoimidazolidine referred to as Cyclocreatine Phosphate (CCrP) when administered in an ISO rat model of HF has shown to prevent the development of HF by:
1. preventing ischemic injury as indicated by normal level of the cardiac biomarker CK-MB after 24 hours;
2. preventing cardiac remodeling by reducing fibrosis and collagen deposition;
3. preventing gain in heart weight; and
4. restoring normal left ventricular ejection fraction and cardiac function, thus, restored high physical activity.

Briefly, CCrP not only prevented ischemia-induced myocardial injury by 24 hours after ISO administration, but also protected cardiac tissue from remodeling and prevented the “progression” of myocardial injury to acute heart failure at day 14. Thus, the bioenergetic CCrP is a promising first-in-class novel mechanism of cardioprotection that prevents ischemic injury, prevents development and progression of heart failure, resulting in rejuvenation of cardiac function and restoration of normal physical activity.

Since, brain and neuro system is very similar in its demand for energy and ATP as the above-discussed heart and cardiac system, where a new paradigm based on the Nourin protein upregulation associated with ischemic events in the heart and cardiac system emerged and is disclosed herein, in which when targeted by the bioenergetic drugs of the present disclosure, namely, 1-carboxy methyl-2-iminoimidazolidine referred to as Cyclocreatine (CCr) and 1-carboxymethyl-2-imino-3-phosphonoimidazolidine referred to as Cyclocreatine Phosphate (CCrP) to prevent and treat such ischemia-induced diseases in the cardiac system, thus, the present disclosure explored, developed and disclose methods for prevention and treatment of ischemia-induced neuro injury and diseases.

Consequently, an aspect of the present disclosure discloses a method for preventative and therapeutic treatment of neuro ischemic diseases and disorders involving ischemia-induced injury, which include aging-related disorders the method comprising the steps of: (a) recruiting a subject; (b) monitoring the subject for presence of ischemic events, ischemia-induced injury, and tissue deterioration by assessing the levels of external markers for the ischemic events, ischemia-induced injury, and tissue deterioration known for characterizing neuro ischemic diseases and disorders involving ischemia-induced injury; (c) collecting a first set of samples from the subject; (d) analyzing the first set of samples and assessing for the expression and release of neutrophil chemotactic factor referred to as Nourin protein levels, for ATP levels, and for the levels of internal markers of the downstream determiners for the ischemic events, ischemia-induced injury, and tissue deterioration selected from inflammation, apoptosis, necrosis, organ failure, and loss of organ function markers known for characterizing neuro ischemic diseases and disorders involving ischemia-induced injury; (e) classifying the monitored subject in terms of the levels of said external markers of step (b), and the levels of said internal markers of step (d) to determine the stage and progress of the ischemic events, ischemia-induced injury, and tissue deterioration in the subject to understand the severity of the neuro ischemic diseases and disorders involving ischemia-induced injury and to calculate a therapeutically effective amount of a bioenergetic agent to be administered to the subject; (f) administrating to the subject the therapeutically effective amount of the bioenergetic agent as calculated in step (e); (g) monitoring the subject again at various time-intervals after said administrating of the bioenergetic agent by assessing the levels of external markers for the ischemic events, ischemia-induced injury, and tissue deterioration known for characterizing neuro ischemic diseases and disorders involving ischemia-induced injury; (h) collecting from the subject a second set of samples after administrating the bioenergetic agent and subsequent sets of samples at various time-intervals after said administrating; (i) analyzing the second set of samples and the subsequent sets of samples of step (h) and assessing for the expression and release of neutrophil chemotactic factor referred to as Nourin protein levels, for ATP levels, and for the levels of internal markers of the downstream determiners for the ischemic events, ischemia-induced injury, and tissue deterioration selected from inflammation, apoptosis, necrosis, organ failure, and loss of organ function markers known for characterizing neuro ischemic diseases and disorders involving ischemia-induced injury; (j) calculating the effectiveness of the bioenergetic agent in terms of the expression and release of neutrophil chemotactic factor referred to as Nourin protein, and the ATP levels by comparing their levels in the first set of samples as analyzed in step (d) to the second set of samples and subsequent sets of samples after said administrating as analyzed in step (i): (k) calculating the presence, progress, and stage of the ischemic events, ischemia-induced injury, and tissue deterioration after said administrating of the bioenergetic agent in terms of the levels of external markers for the ischemic events, ischemia-induced injury, and tissue deterioration as assessed in step (b) in comparison to as assessed after said administrating in step (g), and the levels of internal markers of the downstream determiners for the ischemic events, ischemia-induced injury, and tissue deterioration selected from inflammation, apoptosis, necrosis, organ failure, and loss of organ function markers by comparing their levels in the first set of samples as analyzed in step (d) to the second set of samples and subsequent sets of samples after said administrating as analyzed in step (i) to check the effectivity of the bioenergetic agent in halting or reversing progress, prevention, or treatment of neuro ischemic diseases and disorders involving ischemia-induced injury, wherein the bioenergetic agent is a synthetic analogue that maintains and restores mitochondrial bioenergetics associated with ATP generation disrupted in ischemic events and ischemia-induced injury associated with neuro ischemic diseases and disorders involving ischemia-induced injury, wherein the ischemia-induced injury comprises a tissue in an organ of the subject exposed to injury, hypoxia, or ischemia associated with neuro ischemic diseases and disorders involving ischemia-induced injury, and wherein the ischemia comprises warm, cold, or demand ischemia, wherein the bioenergetic agent preserves mitochondrial biogenesis, prevents cell injury, prevents disease development rejuvenates organ function, and restores normal physical activity, and wherein the bioenergetic agent is selected from 1-carboxymethyl-2-iminoimidazolidine referred to as Cyclocreatine (CCr), and 1-carboxymethyl-2-imino-3-phosphonoimidazolidine referred to as Cyclocreatine phosphate (CCrP).

In another aspect of the present disclosure, it discloses a method for prevention and treatment of ischemic events and ischemia-induced injury and tissue deterioration associated with neurologic surgical procedures, the method comprising the steps of: (i) recruiting a subject set to undergo a neurologic surgical procedure; (ii) monitoring the subject for presence of ischemic events, ischemia-induced injury and tissue deterioration by assessing the levels of external markers for the presence of ischemic events, ischemia-induced injury and tissue deterioration known to be associated with neurologic surgical procedures; (iii) collecting a first set of samples from the subject before the performance of the neurologic surgical procedure that the subject is set to undergo; (iv) analyzing the first set of samples and assessing for the expression and release of neutrophil chemotactic factor referred to as Nourin protein levels, for ATP levels, and for the levels of internal markers of the downstream determiners for the ischemic events, ischemia-induced injury and tissue deterioration selected from inflammation, apoptosis, necrosis, organ failure, and loss of organ function markers known to be associated with neurologic surgical procedures; (v) classifying the monitored subject in terms of the levels of said external markers of step (ii), and the levels of said internal markers of step (iv) to determine the stage and progress of the ischemic events, ischemia-induced injury and tissue deterioration in the subject to understand the condition of the subject set to undergo the neurologic surgical procedure and to calculate a therapeutically effective amount of a bioenergetic agent to be administered to the subject; (vi) administrating to the subject the therapeutically effective amount of the bioenergetic agent as calculated in step (v); (vii) monitoring the subject again at various time-intervals after said administrating of the bioenergetic agent by assessing the levels of external markers for the ischemic events, ischemia-induced injury and tissue deterioration known to be associated with neurologic surgical procedures; (viii) collecting from the subject a second set of samples after administrating the bioenergetic agent and subsequent sets of samples at various time-intervals after said administrating; (ix) analyzing the second set of samples and the subsequent sets of samples of step (viii) and assessing for the expression and release of neutrophil chemotactic factor referred to as Nourin protein levels, for ATP levels, and for the levels of internal markers of the downstream determiners for the ischemic events, ischemia-induced injury and tissue deterioration selected from inflammation, apoptosis, necrosis, organ failure, and loss of organ function markers known to be associated with neurologic surgical procedures; (x) calculating the effectiveness of the bioenergetic agent in terms of the expression and release of neutrophil chemotactic factor referred to as Nourin protein, and the ATP levels by comparing their levels in the first set of samples as analyzed in step (iv) to the second set of samples and subsequent sets of samples after said administrating as analyzed in step (ix), (xi) calculating the presence, progress, and stage of the ischemic events, ischemia-induced injury and tissue deterioration after said administrating of the bioenergetic agent in terms of the levels of external markers as assessed in step (ii) in comparison to as assessed after said administrating in step (vii), and the levels of internal markers as analyzed in step (iv) in comparison to the as assessed after said administrating as analyzed in step (ix) to check the effectivity of the bioenergetic agent in halting or reversing progress, prevention, or treatment of ischemic events, ischemia-induced injury and tissue deterioration associated with neurologic surgical procedures, wherein the bioenergetic agent is a synthetic analogue that maintains and restores mitochondrial bioenergetics associated with ATP generation disrupted in ischemic events, ischemia-induced injury and tissue deterioration associated with neurologic surgical procedures, wherein the ischemia-induced injury comprises a tissue in an organ of the subject exposed to injury, hypoxia, or ischemia associated with neurologic surgical procedures involving ischemia-induced injury, and wherein the ischemia comprises warm, cold, or demand ischemia, wherein the bioenergetic agent preserves mitochondrial biogenesis, prevents cell injury, prevents disease development, rejuvenates organ function, and restores normal physical activity, and wherein the bioenergetic agent is selected from 1-carboxy),methyl-2-iminoimidazolidine referred to as Cyclocreatine (CCr), and 1-carboxymethyl-2-imino-3-phosphonoimidazolidine referred to as Cyclocreatine phosphate (CCrP).

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWING

The accompanying drawings are included to provide a further understanding of the invention, and are incorporated in and constitute a part of the present invention and, together with the description, serve to explain the principle of the invention.

In the drawings,

FIG. 1 is a representation of the proposed mechanism of action of the cardioprotective benefits of Cyclocreatine (CCr) and Cyclocreatine Phosphate (CCrP).

FIG. 2A to FIG. 2B indicate: (A): the chemical structure of Phosphocreatine (CrP) and Cyclocreatine Phosphate (CCrP); and (B): how Cyclocreatine significantly reduced my ocardial cell injury in the intact AMI dog model of LAD occlusion for 1 hour followed by reperfusion for 2 hours.

FIG. 3A to FIG. 3B indicate: (A): Cyclocreatine inhibits levels of Nourin protein in plasma samples in the intact AMI dog model of LAD occlusion for 1 hour followed by reperfusion for 2 hours, as well as reduces neutrophil accumulation into the myocardium after reperfusion for 2 hours: and (B): Cyclocreatine B immediately restores heart contractile function during reperfusion compared to control saline hearts A which never recovered in the intact AMI dog model of LAD occlusion for 1 hour followed by reperfusion for 2 hours.

FIG. 4A to FIG. 4B indicate: (A): much higher neutrophil accumulation after reperfusion in the right and left atria (+2-3) compared to the right and left ventricular (+1) in the intact canine model of cold cardioplegic arrest and aortic cross-clamping for 1 hour followed by reperfusion on bypass for 45 min and then off bypass for 4 hours: and (B): post-bypass cardiac output was significantly better in CCr-treated hearts compared to that of controls, where the CCr-treated hearts achieved over 90% of the baseline function throughout the 4 hours of reperfusion, while control hearts achieved only 60% of the baseline function. Dogs were injected intravenously with saline or CCr (500 mg/kg) for 1 hour before initiating the experiment. Although all control saline-treated dogs required defibrillation to resume cardiac contractility, CCr-treated dogs resumed immediate contractility during reperfusion without the need for defibrillation.

FIG. 5A to FIG. 5B indicate: (A): Cyclocreatine reduces apoptotic enzyme activity in the non-heart-beating dog model of heart transplantation. Dog hearts underwent 1 hour of global warm ischemic arrest then hearts were explanted and perfused Ex vivo for an Additional 4 hours with a cold lactated ringers solution containing Cyclocreatine, while control hearts received cold lactated ringers’ solution alone, and (B): Cyclocreatine Phosphate (CCrP) reduces heart weight after 6 hours of cold storage in HH solution (UW + CCrP) compared to control (UW).

FIG. 6 provides that Cyclocreatine Phosphate protects rat donor hearts against ischemic injury during harvesting and prolonged cold storage for 22 Hours and 24 Hours, as well as after grafting the hearts for 3 days (Saline=5 and CCrP=6). CCrP protection was evident in CCrP grafted hearts at day 3 where the myocardial color and the consistency of the degree of contractility were almost the same as day zero after transplantation. Additionally, the day 3 ECHO showed the continued preservation of the myocardial wall thickness and mass which are the main criteria that determine the degree of myocardial ischemia over a period of time. Most the control grafted hearts, on the other hand, continued to show evidence of ischemia, as well as loss of the wall thickness and the cardiac mass by day 0 and day 3.

FIG. 7A to FIG. 7D present the gene expression level of Nourin RNA network composed of miR-137/mRNA-FTHL-17/ IncR-CTB89H12.4 in the standard isoproterenol (ISO) model of HF and demonstrate how the administration of Cyclocreatine Phosphate (CCrP) inhibited gene expression of Nourin RNA network. (A) and (B): indicate the significantly high gene expression level of miR-137 in serum samples collected at day 14 from ISO/saline rats compared to normal rats. The ISO/saline rats had upregulation of miR-137 by 8.91-fold (Mean=10.25) compared to healthy rats received saline (1.15) (p<0.0001), where there was none to a minimal gene expression of miR-137 in normal non-stressed rats. CCrP treatment significantly (p<0.0001) reduced miR-137 gene expression at doses of 0.4 g/kg, 0.8 g/kg and 1.2 g/kg by 33%, 75% and 68%, respectively. Additionally, CCrP administration to healthy rats at 0.8 g/kg did not increase miR-137 gene expression (Mean=1.60) and it was comparable to the level expressed in saline-treated healthy rats (1.15); (C): The ISO/saline rats had upregulation of mRNA-FTHL-17 by 8.17-fold (Mean=8.26) compared to healthy rats received saline (1.01) (p=0.0002). CCrP treatment significantly (p=0.04) reduced mRNA-FTHL-17 gene expression at doses of 0.4 g/kg, 0.8 g/kg and 1.2 g/kg by 16%, 30% and 75%, respectively. Additionally, CCrP administration to healthy rats at 0.8 g/kg did not increase mRNA-FTHL-17 gene expression (Mean=0.67) and it was comparable to the level expressed in saline-treated healthy rats (1.01); and (D): The ISO/saline rats had downregulation of IncR-CTB89H12.4 (Mean=0.3) compared to healthy rats received saline (1.1) (p=0.002). CCrP treatment significantly (p=0.002) increased IncR-CTB89H12.4 gene expression at doses of 0.4 g/kg. 0.8 g/kg and 1.2 g/kg by 1.33-fold. 7.66-fold and 14.33-fold, respectively. Additionally, CCrP administration to healthy rats at 0.8 g/kg had a comparable IncR-CTB89H12.4 gene expression level (Mean=1.3) as the saline-treated healthy rats (1.1). Results suggest lack of cardiac toxicity by CCrP.

FIG. 8 is a representation of the correlation analysis was conducted between miR-137/mRNA-FTHL-17/IncRNA-CTB89H12.4 in the ISO/saline rats treated with ISO/CCrP at 0.8 g/kg. The only significant correlation was found between miR-137 and IncR-CTB89H12.4 (p=0.04) in ISO/CCrP group. No significant correlation was detected between miR-137/ mRNA-FTHL-17/ lncR-CTB89H12.4 in the ISO group (p>0.05).

FIG. 9A to FIG. 9D present the gene expression level of Nourin RNA network composed of miR-106b/mRNA-ANAPC11/ lncR-CTB89H12.4 in the standard isoproterenol (ISO) rat model of HF and how the administration of Cyclocreatine Phosphate (CCrP) inhibited gene expression of Nourin RNA network. (A) and (B): indicate the significantly high gene expression level of miR-106b in serum samples collected at day 14 from ISO/saline rats compared to normal rats. The ISO/saline rats had upregulation by 8.74-fold (Mean=40.38) compared to healthy rats received saline (4.62) (p<0.0001). CCrP treatment significantly (p<0.001) reduced miR-106b gene expression at doses of 0.4 g/kg, 0.8 g/kg and 1.2 g/kg by 18%, 44% and 72%, respectively. Additionally, CCrP administration to healthy rats at 0.8 g/kg did not increase miR-106b gene expression (Mean=5.62) and it was comparable to the level expressed in saline-treated healthy rats (4.62); (C): ISO/saline rats had upregulation of mRNA-ANAPC11 by 101.4-fold (Mean=101.4) compared to healthy rats received saline (1.0) (p=0.0002). CCrP treatment significantly (p=0.04) reduced mRNA-ANAPC11 gene expression at doses of 0.4 g/kg, 0.8 g/kg and 1.2 g/kg by 18%, 31% and 70%, respectively. Additionally, CCrP administration to healthy rats at 0.8 g/kg did not increase mRNA-ANAPC11 gene expression (Mean=0.9) and it was comparable to the level expressed in saline-treated healthy rats (1.0): and (D): The ISO/saline rats had downregulation of lncR-CTB89H12.4 (Mean =0.3) compared to healthy rats received saline (1.1) (p=0.002). CCrP treatment signicantly (p=0.002) increased IncR-CTB89H12.4 gene expression at doses of 0.4 g/kg. 0.8 g/kg and 1.2 g/kg by 1.33-fold, 7.66-fold and 14.33-fold, respectively. Additionally, CCrP administration to healthy rats at 0.8 g/kg had IncR-CTB89H12.4 gene expression (Mean=1.3) had a comparable level of expression as the saline-treated healthy rats (1.1). No significant correlation was detected between miR-106b/ mRNA-ANAPC11/ lncR-CTB89H12.4 in the ISO/saline group (p>0.05). Similarly, no significant correlation was detected between miR-106b/ mRNA-ANAPC11/ lncR-CTB89H12.4 in the ISO/CCrP group (0.8 g/kg) (p>0.05).

FIG. 10A to FIG. 10D present the cardioprotective benefits of CCrP administration in the standard isoproterenol (ISO) rat model of HF by preventing the development of HF and restoring normal cardiac function of ejection fraction (EF%) measured by ECHO analysis. EF is an important measurement of how well the heart is pumping, and it is used to help classify heart failure and guide treatment. FIG. 10 indicates: (A): ejection fraction; (B): cardiac biomarker CK-MB; (C): collagen deposition; and (D): heart weight.

FIG. 11 indicates the safety of CCrP at a dose of 0.8 g/kg, injected IP daily to healthy rats for 14 days and showed no toxicity in liver and renal function. There was no significant difference between normal rats treated with saline or CCrP for the levels of liver enzyme ALT. kidney Creatinine and Urea Similarly, the expression level of Nourin RNA network (miR-137, miRNA-106b, mRNA-FTHL-17, mRNA-ANAPC11, and lncR-CTB89H12.4) was comparable in healthy rats treated with saline or CCrP (FIG. 7A - FIG. 7D and FIG. 9A - FIG. 9B). These results suggest lack of toxicity by CCrP.

FIG. 12A and FIG. 12B are photos of a representative rat from: (A) ISO/saline group (n=6) with a “low physical activity” at day 14 before sacrifice where rats primarily stayed in place (FIGS. 12A); and (B): ISO/CCrP group (n=5) showed “high physical activity” at day 14 before sacrifice, which is comparable to normal healthy control “saline” rats (FIG. 12B). These results indicate that treating ISO rats with CCrP prevented the development of heart failure and restored normal heart function and physical activity.

FIG. 13 indicates a summary flow diagram at day 14 illustrating that ischemia-induced ISO rats which developed HF show upregulation of Nourin-associated miR-137 (marker of cell damage) and miR-106b-5p (marker of inflammation) with a likely increase in translation and production of the Nourin protein. The diagram also indicates amelioration of myocardial ischemic injury and the molecular regulation of the Nourin protein when rats were treated with the cardioprotective compound, CCrP, in rats that did not develop HF. ISO: isoproterenol, CCrP: cyclocreatine phosphate.

FIG. 14 indicates a summary flow diagram illustrating that the administration of CCrP prevented ischemic injury and resuscitated poorly functioning hearts at the early acute phase of 24 hours after the second ISO injection, as shown by normal ECG/ST and CK-MB levels. After 14 days, CCrP prevented the development of HF as indicated by a reduction of apoptosis, inflammation, biomarkers, cardiac remodeling (fibrosis/collagen deposition), and heart weight, with the restoration of normal ejection fraction, cardiac function, and physical activity in ISO/CCrP rats. ISO: isoproterenol; CCrP: Cyclocreatine phosphate.

FIG. 15A and FIG. 15B indicate: FIG. 15(A) shows that CCrP prophylactically-administered in rats before the first ISO injection, prevents myocardial ischemic injury and cardiac dysfunction measured 24 hours after the second ISO injection. FIG. 15(B) shows that CCrP therapeutically administered in rats after the second ISO injection, salvages poorly functioning hearts. Data are presented in mean ± S.E.M. *: statistical significance (p<0.05) compared to the saline/control group. #: statistical significance (p<0.05) compared to the ISO+saline group.

FIG. 16A and FIG. 16B indicate: FIG. 16A shows that CCrP prophylactically administered before the first ISO injection continued to prevent myocardial injury and the development of cardiac dysfunction in terms of ejection fraction percent (EF%) and FIG. 16B in terms of the heart weight in terms of heart weight index (HWI) at the end of the 14-day treatment. Data are presented in mean ± S.E.M. *: statistical significance (p<0.05) compared to the saline/control group. #: statistical significance (p<0.05) compared to the ISO+saline group.

FIG. 17 indicates ECHO images demonstrate the effect of CCrP on ISO-induced changes in M-mode in LVEDD (yellow arrow) and LVESD (green arrow). Groups include saline/control (panel A), CCrP/control (panel B), ISO/Saline (panel C), and ISO+CCrP (panel D). CCrP at a dose of 0.8 g/kg/day.

FIG. 18A and FIG. 18B indicate that CCrP reduced cardiac inflammation and remodeling evaluated in terms of Fibrin and Collagen deposition. FIG. 18A indicates that histopathological analysis of H&E (cardiac inflammation) and Masson’s trichrome stained (cardiac fibrin and collagen deposition) of the effect of different doses of CCrP on myocardial fibrosis associated with ISO administration. Specimens stained with Masson’s trichrome for estimation of myocardial fibrosis (blue color) in saline/control, CCrP/control, ISO/saline, and ISO/CCrP at a dose of 0.8 g/kg/day. FIG. 18B indicates that ISO/CCrP at a dose of 0.8 g/kg showed an 83% reduction in collagen% compared to levels detected in the ISO/saline rats. Data are presented in mean ± S.E.M. *: statistical significance (p<0.05) compared to the saline/control group. #: statistical significance (p<0.05) compared to the ISO+saline group.

FIG. 19A to FIG. 19F indicate cardiac biomarkers and protein expression showing that CCrP restores cardiac biomarkers and protein expression. FIG. 19A indicates for TNF-a: ISO/CCrP at a dose of 0.8 g/kg/day showed a significant reduction of TNF-α compared to ISO/saline rats; FIG. 19B indicates for TGF-β: ISO/CCrP showed a significant reduction of TGF-β compared to ISO/saline rats: FIG. 19C indicates for Tn I: ISO/CCrP showed a significant reduction in Tn I compared to ISO/saline rats: FIG. 19D indicates for Caspase-3: ISO/CCrP showed a significant reduction in Caspase-3 contents compared to ISO/saline rats; FIG. 19E indicates for eNOS: ISO/CCrP showed a significant increase in eNOS contents compared to ISO/saline rats: and FIG. 19F indicates for connexin-43 β-actin: ISO/CCrP showed a significant increase in connexin-43 β-actin contents compared to ISO/saline rats. Data are presented in mean ± S.E.M. *: statistical significance (p<0.05) compared to the saline/control group. #: statistical significance (p<0.05) compared to the ISO+saline group.

FIG. 20A to FIG. 20C indicate that therapeutically-administered CCrP salvages poor heart function and sustains normal cardiac function. FIG. 20A: ISO/saline showed a significant reduction of EF%, which was restored to normal levels by the therapeutic treatment of CCrP at the dose of 0.8 g/kg/day; FIG. 20B: CCrP treatment also significantly reduced levels of Tnl; and FIG. 20C: BNP. Data are presented in mean ± S.E.M. *: statistical significance (p<0.05) compared to the saline control group. #: statistical significance (p<0.05) compared to the ISO+saline group.

FIG. 21A and FIG. 21B indicate that the treatment of CCrP at the dose of 0.8 g/kg/day restores normal physical activity in both prophylactic (a) and therapeutic (b) regimens. Data are presented in mean ± S.E.M. *: statistical significance (p<0.05) compared to the control/saline group, #: statistical significance (p<0.05) compared to the ISO + saline group.

FIG. 22 indicates cardiac Nourin protein expression and provides that high elevated level of Nourin protein expression was observed due to ischemic injury treated with creatine or buffer, which was remarkably reduced by the treatment with Cyclocreatine (CCr), whether perfused or incubated suggesting inhibition of cardiac Nourin by said CCr treatment

FIG. 23 indicates the release of Nourin protein from isolated bovine coronary artery segments.

FIG. 24 indicates the release of Nourin protein from isolated human vein grafts.

FIG. 25A to FIG. 25C indicate administration of Cyclocreatine (CCr) shortly prior to the induction of ischemia via aortic cross-clamping effective in restoring post-ischemic nerve function. Here. CCr function was evaluated 24 hours post-operatively using standard Tarlov Scale, which is illustrated graphically for 3 days prior-treatment before ischemic condition in FIG. 25A, 4 days prior-treatment before ischemic condition in FIG. 25B. and combined result in FIG. 25C.

FIG. 26 indicates the release of rapid release of Nourin protein by ischemic pig spinal cord in response to the ischemic injury.

FIG. 27 indicates the release of rapid release of Nourin protein by pig brain in response to ischemic injury.

FIG. 28 indicates high levels of the retinal chemotactic factor Nourin being released after 1 hour and 6 hours in response to ischemic and oxidative injury in levels higher than the positive control f-MLP.

FIG. 29 indicates a schematic representation of mechanism of ischemic injury produced endpoints of ischemia including, inflammation, apoptosis, necrosis, organ failure and loss of function in the organ in ischemic diseases and aging related disorders involving ischemia injury, where Nourin protein is upregulated in response to said ischemic injury and a new paradigm based on Nourin protein up-regulation in response to ischemic injury in high energy or ATP requiring organs particularly the heart or cardiac system and the brain or neuro system and in aging-related disorders involving ischemia injury. Further, it represents targeting the same with the use of cyclocreatine (CCr) and/or cyclocreatine phosphate (CCrP), where targeting the same with the use of cyclocreatine (CCr) and/or Cyclocreatine phosphate (CCrP) owing to its anti-inflammatory and anti-apoptotic functions is key in ultimately protecting against the aforementioned effects leading to loss of organ function.

FIG. 30 indicates a schematic representation of a new paradigm based on Nourin protein up-regulation in response to ischemic injury in high energy or ATP requiring organs particularly the heart or cardiac system and the brain/spinal cord/ocular or neuro system and in aging-related disorders involving ischemia injury and targeting the same with the use of cyclocreatine (CCr) and/or cyclocreatine phosphate (CCrP) to counter the depletion of ATP in such organs to combat and prevent consequent cell injury, inflammation, apoptosis, and ultimate loss of organ function.

FIG. 31 indicates a schematic representation showing the molecular mechanisms underlying (oxidative stress: reactive oxygen species (ROS))-, (inflammation)-, and (dysfunctional mitochondrial bioenergetics associated with ATP generation disruption)- mediated deregulation in neuro ischemic diseases and disorders involving ischemia-induced injury of central nervous system (CNS) as disclosed in the present disclosure based on the paradigm developed and presently disclosed in the cardiovascular disease (CVD) system.

### DETAILED DESCRIPTION OF THE INVENTION

The present invention may be understood more readily by reference to the following detailed description of the invention taken in connection with the accompanying drawing figures, which forms a part of this disclosure. It is to be understood that this invention is not limited to the specific devices, medicines, systems, conditions or parameters described and/or shown herein and that the terminology used herein is for the example only, and is not intended to be limiting of the claimed invention. Also, as used in the specification including the appended claims, the singular forms ‘a’, ‘an’, and ‘the’ include the plural, and references to a particular numerical value includes at least that particular value unless the content clearly directs otherwise . Ranges may be expressed herein as from ‘about’ or ‘approximately’ another particular value. When such a range is expressed, it is another embodiment. Also, it will be understood that unless otherwise indicated, dimensions and material characteristics stated herein are by way of example rather than limitation, and are for better understanding of sample embodiment of suitable utility, and variations outside of the stated values may also be within the scope of the invention depending upon the particular application.

Embodiments will now be described in details with reference to the accompanying drawings. To avoid unnecessarily obscuring in the present disclosure, well-known features may not be described, or substantially the same elements may not be redundantly described, for example. This is for ease of understanding. The drawings and the following description are provided to enable those skilled in the art to fully understand the present disclosure and are in no way intended to limit the scope of the present disclosure as set forth in the appended claims.

Tissue-Derived Nourin: The host-derived formyl peptide Nourin is a 3 KDa proinflammatory protein belongs to a family of N-formyl methionyl chemotactic peptides with a common motif of formyl-methionyl at the N-terminus. Nourin acts through the formyl peptide receptor (FPR) on leukocytes. Nourin is a potent “EARLY” formyl peptide inflammatory mediator released by local tissues in response to “reversible” and “irreversible” ischemic injury, as well as infections (1-40). Nourin expression also continues as injury is sustained.

The Nourin family represents the “initial signal” in the cascade of events that lead to acute and chronic inflammation and tissue injury. Specifically. Nourin “recruits” leukocytes (neutrophils, monocytes) to injured sites and “activates” the cells to secrete adhesion molecules, chemokines, cytokines (ICAM-1, ELAM-1, LECAM, IL-8, IL-1β, and TNF-α), and toxic substances such as oxidants and digestive enzymes. Nourin also stimulates the recruitment and activation of vascular endothelial cells. Since Nourin is released by local tissues following injury and it contributes to the induction of inflammatory responses, it can be characterized as an Alarmin and used as a crucial therapeutic target.

Chemotaxis Assay: The chemotactic activity of Nourin protein in different samples which was described in the below examples was tested using standard chemotaxis assay [A, B]. Briefly, neutrophils were isolated from human peripheral blood and labeled by fluorescence dye. These labeled neutrophils were used as migratory cells and placed on the top chamber of 96 well chemotaxis plates. Samples were placed at the bottom wells of the plate . A filter was placed in between and the ability of the tested samples to stimulate the migration of neutrophils across the membrane was determined by incubating the chamber at 37° C. for 1 hour. The standard synthetic chemoattractant f-Met-Leu-Phe (f-MILF (SEO ID NO: 1)) was used as the positive control for 100% chemotactic response. Hank’s Balanced Salt Solution (HBSS) was the negative control for random migration. Neutrophil migration was reported as the number of label 5 filter in response to the test solutions. The samples were tested neutrophils detected at the bottom wells which crossed the membrane in triplicate wells.

As discussed in the background above, currently there are no effective preventative or therapeutic treatment options for ischemic diseases and disorders involving ischemia-induced injury of the neuro system, the present invention explored addressing the same. Heart and brain are among parts of the body requiring the greatest amounts of energy and they are the most affected during failures of the mitochondria to generate sufficient cellular adenosine triphosphate (ATP) during hypoxia and ischemia. Both organs are sensitive to changes in oxygen supply and ischemia and inflammation play a key role in acute stroke and acute myocardial infarction. Similarly, spinal cord ischemia is associated with a loss of neuromuscular function, and retinal and optic nerve ischemia results in vision loss.

ATP depletion in ischemic heart and similarly in the brain, is a critical contributor to the pathogenic event that triggers cell injury, inflammation/fibrosis, apoptosis, leading to organ dysfunction. With aging, a decline in mitochondrial function and a reduction of oxygen supply associated with vascular dysfunction, increases the incidence of heart failure (HF), stroke, and Alzheimer Disease (AD). Although the cause of AD remains uncertain until now, it is defined pathologically by mitochondrial dysfunction and loss of ATP, as well as by loss of synaptic and neuronal function due to the downstream accumulation of amyloid-beta peptide (Aβ) plaques and tau protein triggered by oxidative stress and inflammation. Additionally, while no effective treatment exists, current therapy provides symptomatic relief without being able to prevent, stop, or reverse the pathologic process of AD. Currently, no effective treatment exists for AD patients, but available therapies provide symptomatic relief without being able to prevent, stop, slow, or reverse the disease. No prior studies have addressed the problem of inefficient production of ATP within the neural tissue by directly increasing ATP supply via the phosphocreatine system. Metabolic diseases such as diabetes result in vascular damage and reduction of sufficient blood flow to tissues, leading to the development of a number of ischemia-related diseases such as diabetic retinopathy (leading cause of blindness worldwide), peripheral artery disease (PAD), optic nerve ischemia, and cerebrovascular diseases. Injury to the vasculature also results in ischemia of nerve tissue and the development of many neurological diseases. Hydrogen peroxide as some-times released as an injurious agent to tissues leading to the release of several cytokines, including Nourin from tissues such as retina, spinal cord and brain. The present invention can protect the vasculatures from damage secondary to diabetes. Thus, there is an unmet need to develop new therapeutic approaches as bioenergetic therapy to preserve the energy source and prevent the development and reduce neuro and neuromuscular disease progression.

While majority of existing treatment paradigm for AD targets the downstream accumulation of Aβ and tau proteins, CCrP approach is a new paradigm that targets upstream ATP by enabling energy stores to be maintained even during reduction of blood reperfusion and tissue oxygenation, thus prevents ischemic injury and blocks the common pathways of cell injury, inflammation, apoptosis that can lead to protein accumulation and brain dysfunction.

A promising way to enhance myocardial tissue ATP stores is via the creatine kinase pathway using the high energy phosphate donor, cyclocreatine phosphate (CCrP). When given prophylactically in a variety of animal models (demand ischemia, spinal cord injury, and others), CCrP was shown to prevent myocardial injury and maintain cardiac contractility. A unique advantage of exploiting the creatine kinase pathway as compared to other therapeutic targets is that it enables ATP to be restored even in the presence of active hypoxia and demand ischemia. In the present disclosure, CCrP was administered therapeutically, i.e., after causing demand ischemia, ATP depletion and myocardial dysfunction in an isoproterenol rat model to simulate a brain-dead donor. The present invention thus hypothesized that restoration of ATP levels after demand ischemia by CCrP will prevent myocardial injury, resuscitate poor heart function, and sustain long-term restoration of EF%.

Heart failure-related left ventricular remodeling is a complex process involving cardiac myocyte death, fibrosis, inflammation, ventricular remodeling, and loss of contractile activity. CAD is a leading cause of HF and that LV remodeling is derived mainly from patients of myocardial infarction. In response to ischemic/reperfusion injury, cardiomyocyte loss is through cell death pathways such as necrosis, apoptosis, or possibly excessive autophagy.

Cardiac remodeling refers to a progressive series of changes in the size, shape, and function of the heart that are initiated by damage to the myocardium or increases in wall stress. Remodeling is a major factor in the development and progression of HF. It involves changes in both the cardiomyocytes and the makeup of the extracellular matrix (ECM). The latter consists of an intricate weave of (predominantly) collagen fibrils that play a vital role in maintaining the structural and functional integrity of the heart.

The immune system plays a significant role in ventricular remodeling, and its persistent activation may lead to long-term cardiac injury. In the next stage of infarct healing, ischemically injured and dying cardiac myocytes release intracellular proteins such as the cardiac-derived inflammatory mediator, Nourin into the circulation and trigger an inflammatory response. Inflammatory cells, including neutrophils, monocytes, macrophages, and lymphocytes infiltrate the tissue. These immune cells remove dead myocytes and pave the way for healing. After resolution of the inflammatory response, cardiac fibroblasts proliferate and secrete extracellular matrix proteins such as collagen 1 to form a fibrotic scar that replaces dead myocytes. The resulting tightly cross-linked, fibrotic scar with significant tensile strength serves to prevent rupture. This remodeling of the LV continues progressively in response to increases in wall stress, provoking cardiac myocyte hypertrophy in the infarct border zone, wall thinning, and chamber dilation. This global adverse remodeling response leads to increases in both LV end-diastolic and end-systolic volumes and reduced ejection fraction.

Ventricular remodeling is also a deposition of excessive extracellular matrix. This surplus extracellular matrix, which constitutes scar or fibrosis, promotes both contractile dysfunction and rhythm disturbances. As a result, cardiac fibrosis contributes to morbidity and mortality in many forms of heart disease. Indeed, the amount of fibrotic scar in the myocardium correlates strongly with the increased incidence of arrhythmias and sudden cardiac death. Extracellular matrix deposition and fibrosis formation occur through the action of cardiac fibroblasts. In the setting of pathological stress, fibroblasts proliferate and differentiate into myofibroblasts, thereby gaining the capacity to contract and secrete collagen I, collagen III, and fibronectin. Within the LV facilitate, both collagenous and myofibroblasts propagate the arrhythmic phenotype of the remodeled heart

Cardiac fibrosis is an independent and predictive risk factor for heart failure. Some evidence suggests that the modulation of cardiac fibrosis alters the arrhythmic phenotype in patients with heart disease. To date, no therapeutic strategy has been developed to specifically target fibrosis in the heart.

The immune system also plays a significant role in ventricular remodeling, and its persistent activation may lead to long-term cardiac injury. The upregulation of miR-106b promotes cardiomyocyte inflammation, which may be an early regulatory mechanism. MicroRNAs involve in the pathophysiological progress in heart failure and it is expected that microRNAs will be widely used in heart failure diagnosis and therapy.

There is a role of biomarkers in HF in conjunction with the clinical and physical assessment. Biomarkers can provide greater diagnostic accuracy than the physical assessment alone. The diagnostic strength of natriuretic peptides is their high sensitivity for “ruling out” HF; however, as the value increases, HF becomes more likely. Defining “rule-in” cutoffs for HF is complicated because multiple factors influence natriuretic peptide levels. The natriuretic peptides are released by the heart in response to myocardial tension and increased intravascular volume and provide accurate tests for the diagnosis of heart failure compared with echocardiography. Brain natriuretic peptides (BNP) and Troponins are the benchmark biomarkers used for the stratification of risk of cardiac dysfunction in patients with AMI. In addition to Troponins as markers of myocardial cell death and BNPs as markers of hemodynamic cardiac stress, other biomarkers of different pathogenetic pathways have been reported. These include: cardiac fibrosis (especially galectin-3), inflammation (C-reactive protein (CRP), growth differentiation factor-15 (GDF-15), osteoprotegerin and extra-cardiac involvement (red blood cell distribution width (RDW)). However, studies indicated that during the early phase of myocardial ischemia, the prognostic value of emergent biomarkers for new-onset HF or deterioration of cardiac function in patients with AMI, suggesting that in most cases, the use of these diagnostic biomarkers of cardiac dysfunction does not translate into efficient risk prediction of HF.

The natriuretic peptides are the best-established and best-evaluated markers to help in the proper diagnosis and exclusion of HF. Natriuretic peptides have led the way as a diagnostic and prognostic tool for the diagnosis and management of HF They can provide important information about disease severity and help in the detection, diagnosis, prognosis, and management of HF. Monitoring their concentrations in blood not only can provide the clinician information about the diagnosis and severity of HF but also can improve prognostication and treatment strategies. However, there is still a critical need for novel diagnostic biomarkers and new therapeutic interventions to decrease the incidence of HF. Inflammatory markers have, also, been evaluated for predicting new-onset HF. In the ABC study (Health, Aging, and Body Composition), IL-6, tumor necrosis factor-α, and CRP were associated with new-onset HF, but when all 3 markers were added to the model, IL-6 emerged as the strongest marker.

Although current therapies for heart failure patients include, angiotensin converting enzyme (ACE) inhibitors, angiotensin receptor blockers (ARBs), aldosterone antagonists, and β-adrenergic receptor blockers (β-blockers), which manifest significant efficacy in reducing morbidity and mortality in patients with chronic systolic heart failure. However, in many instances, disease progression continues unabated. Additionally, novel disease targets are continually being discovered, however, most therapeutics do not demonstrate consistent efficacy in patients: many prove to be ineffective, even deleterious, before reaching Phase III clinical trials.

Cardiac fibrosis is an independent and predictive risk factor for heart failure. Some evidence suggests that the modulation of cardiac fibrosis alters the arrhythmic phenotype in patients with heart disease. To date, no therapeutic strategy has been developed to specifically target fibrosis in the heart. Cardiac remodeling refers to a progressive series of changes in the size, shape, and function of the heart that are initiated by damage to the myocardium or increases in wall stress. Remodeling is a major factor in the development and progression of HF. It involves changes in both the cardiomyocytes and the makeup of the extracellular matrix (ECM). The latter consists of an intricate weave of (predominantly) collagen fibrils that play a vital role in maintaining the structural and functional integrity of the heart. Thus, targeting the reduction of fibrosis and collagen synthesis by the bioenergetic CCrP (Example 4 and Example 5), is a new therapeutic approach for the prevention and treatment of HF.

It was shown that suppression of myocardial contractility plays an important role in the development of heart failure; therefore, there is a need for cardiotonic agents to improve the contractile function of the failing heart. Additionally, studies indicated that the development and progression to HF are associated with a decline in energy reserve capacity that ultimately reaches a threshold after which compensatory mechanisms can no longer support the decreasing energy supply. Growing evidence indicates that derangements in myocardial fuel metabolism and bioenergetics contribute to the development of heart failure. Stored myocardial high-energy phosphate (phosphocreatine) are reduced in humans with pathological ventricular hypertrophy, with further decline during the transition to heart failure. Notably, the [phosphocreatine]/[ATP] ratio correlates with heart failure severity and is a strong predictor of cardiovascular mortality, Thus, targeting energy metabolic disturbances and corresponding upstream regulatory events occurring during the early stages of HF is an important first step toward the identification of new therapeutic targets to improve the outcomes of current therapies. Mitochondrial energy source could, therefore, be a promising therapeutic target to improve mitochondrial biogenesis. Currently, there are no drugs that specifically target mitochondrial biogenesis in HF patients.

Mitochondrial abnormalities and reduced capacity to generate ATP can have a profound impact in heart failure. Abnormal mitochondria are also linked to myocyte injury because they are a major source of reactive oxygen species (ROS) production that can induce cellular damage. Abnormal mitochondria promote programmed cell death through the release of cytochrome c into the cytosolic compartment and activation of caspases. Bendavia was reported to improve cellular ATP levels and prevent pathological ROS formation. However, in the EMBRACE STEMI (Evaluation of Myocardial Effects of Bendavia for Reducing Reperfusion Injury in Patients with Acute Coronary Events-ST-Segment Elevation Myocardial Infarction) trial, Elamipretide did not improve the primary or secondary outcomes. In the randomized placebo-controlled trial of Elamipretide in HF. the drug was shown to reduce left ventricular volumes; however, the confidence intervals were wide in this small study, and there were no changes in biomarker data. Elamipretide is currently being investigated in larger HF studies to determine its effect on cardiac remodeling and clinical outcomes.

Therefore, there is an urgent need for effective new therapeutic drugs which provide protection of heart muscle for HF patients experiencing myocardial ischemia and, thus, save ischemic muscles from progressing to necrosis and heart failure. Saving heart muscles from progressing to permanent damage will particularly be crucial for the outcome of AMI patients who are undergoing angioplasty/percutaneous coronary intervention (PCI) to reduce their progression to heart failure (HF). Depending on the infarct size, up to 50% of AMI patients will proceed to suffer from HF, which is known for its devastating disability. Cyclocreatine Phosphate is a novel mechanism that has the ability to save the reversible ischemic cardiac muscles from progressing to permanent necrosis and, thus, improve AMI patients’ outcome (reducing disability associated with HF), quality of life and patients’ financial burden.

Preservation of mitochondrial energy metabolism by Cyclocreatine Phosphate (CCrP) is a novel therapeutic target potential which can also be applied in a number of additional “ischemic” conditions including: atrial fibrillation (AF). Takotsubo cardiomyopathy, cardiac surgeries, stroke, and Alzheimer’s disease. Currently, there are no drugs specifically target mitochondrial biogenesis in these ischemia-related diseases. Preservation of the energy source ATP, will present a promising therapeutic approach to prevent the development, as well as, treat HF patients. This invention demonstrated that healthy rats treated with CCrP (0.8 gm/kg) for 14 days, showed no toxicity in heart, liver and renal function. Since CCrP showed strong cardioprotective activities against ischemic heart diseases (AMI, bypass, heart transplantation and HF), CCrP can also be useful to prevent and treat other cardiac ischemic diseases (e.g., atrial fibrillation, Takotsubo cardiomyopathy and cardiac surgeries including valve replacement), neurodegenerative diseases which may be aging-related (e.g., cerebral ischemic stroke and Alzheimer), ocular ischemia-induced injury and diseases, optic nerve ischemia-induced injury and diseases, and retinal ischemia-induced injury and diseases.

The present invention demonstrates that healthy rats in the examined ISO model experiments discussed hereinbelow, the rats treated with CCrP at an effective dose of 0.8 gm/kg for 14 days, showed no toxicity in heart, liver and renal function.

Cerebral Ischemia (Stroke) - ischemia and subsequent reperfusion is known to induce irreversible tissue damage with the consequence of more or less pronounced impairments. The prevalence rate of stroke in U.S. was estimated to be approximately 1 in 59 or 1.69% or 4.6 million people. Cerebral ischemia with the resulting strokes is considered as one of the three major causes of death in all countries. In addition, the increase in the number of individuals with physical or mental handicaps after stroke presents considerable problems in terms of quality of life and socioeconomic costs. Despite recent medical and surgical advances, the general approach to prevent acute ischemic brain damage remains inadequate. Up till now, there are no clinical effective protocol for amelioration of brain damage caused by ischemia and reperfusion. Although over the last decades, significant progresses have been made in the development of thrombolytic therapies for acute ischemic stroke, these thrombolytic therapies have restrictive time window. In addition, these therapies have increased risk of cerebral hemorrhage which limits their application for certain patients. Clearly, there is a crucial need to develop neuroprotective new therapies to prevent and treat acute ischemic brain which could be implemented alone or in combination with thrombolytic approaches to improve clinical outcome of more patients with acute ischemic stroke. CCrP can be administrated prophylactically to high-risk patients including aging population to protect against brain ischemic injury. CCrP can also be administered immediately after an ischemic event to protect against deterioration of areas adjacent to ischemic tissues, thus minimize cell injury, loss of function and disability.

Heart and brain are among parts of the body requiring the greatest amounts of energy and they are the most affected during failures of the mitochondria to generate ATP in aging due to diminished vascularization that leads to hypoxia and ischemia. Thus, preservation of the energy source ATP by CCrP. will also present a promising therapeutic approach as a new “age-modifier therapy” to prevent the development and to treat Alzheimer’s disease (AD) similar to HF (described in this invention).

Alzheimer’s disease (AD) - is one of the most common neurodegenerative diseases in the elderly, affecting 40 million people worldwide. The prevalence of AD is strongly correlated with age, imposing a greater socioeconomic burden as life expectancy continues to increase. Recent estimates predict that in the next four decades, the world’s proportion of people aged 65 years and older will account for nearly 22% of the total population-from the present 800 million to 2 billion people. Although this increase in life expectancy is reflective of the healthcare achievements, the socioeconomic costs associated with a higher chronic disease burden have necessitated the development of robust prevention and management strategies that are both safe and immediately executable.

Due to the high energy demands of neurons and glia, a considerable amount of ATP is consumed in the brain. Also, because no energy storage (such as fat or glucose) is available in the central nervous system (CNS), brain cells must continually produce ATP to maintain activity and energy homeostasis. With aging, oxygen delivery to cells and tissues is impaired due to diminished vascularization, thereby increasing the susceptibility of neurons to damage. Thus, hypoxic (neuronal) adaptation is significantly compromised during aging. Many neurological diseases, such as stroke and Alzheimer’s disease (AD) are characterized by hypoxia, a state that is believed to only exacerbate disease progression. AD is a pressing public health problem with no effective treatment. Existing therapies only provide symptomatic relief without being able to prevent, stop or reverse the pathologic process. While the molecular basis underlying this multifactorial neurodegenerative disorder remains a significant challenge, mitochondrial dysfunction appears to be a critical factor in the pathogenesis of this disease. It is therefore important to target mitochondrial dysfunction in the prodromal phase of AD to slow or prevent the neurodegenerative process and restore neuronal function. The relationship between hypoxia and AD could open the avenue for effective preservation and pharmacological treatments of this neurodegenerative disease by using the novel bioenergetic drug, CCrP. It has been previously demonstrated that CCrP crosses blood brain barrier and functions as a potent neuroprotective agent by preventing ischemic injury and restoring organ function. Additionally, similar to heart tissue. Nourin protein was quickly released within 2 minutes by brain and spinal cord tissues in response to ischemia. Because of the great similarities between heart and brain and that both require high demand of ATP, the administration of CCrP will be as effective in preventing ischemic injury and restoring neurologic function in stroke and AD, similar to what has been previously demonstrated in ischemia-induced HF. which can be aging-related.

Clinically, AD is associated with the progressive loss of essential cognitive functions and progressive hippocampal and cortical brain atrophy. Death occurs, on average, 9 years after diagnosis. AD is pathologically defined by the widespread brain distribution of amyloid-beta peptide (Aβ) plaques, neurofibrillary, tangle (NFT) formation, as well as synaptic and neuronal loss. Despite growing understanding of the disease, it remains unclear how these pathological features relate to the specific disease processes. The amyloid cascade hypothesis continues to serve as the predominant model of AD pathology. This hypothesis suggests the overproduction of Aβ as the causal trigger in the disease process. Aβ is derived from the amyloidogenic cleavage of the amyloid precursor protein (APP), protein cleaved by two endoproteases. The disease progression is associated by the accumulation of Aβ peptides and other misfolded proteins such as tau protein (microtubule-associated protein). The accumulation of these peptides eventually leads to cell death and its associated manifestations such as dementia and behavioral changes. These manifestations are brought about due to the triggering of oxidative stress and inflammation. Increasing evidence suggests that hypoxia facilitates the pathogenesis of AD through accelerating the accumulation of Aβ, increasing the hyperphosphoration of tau, impairing the normal functions of blood-brain barrier, and promoting the degeneration of neurons. Additionally, similar to the ischemia-induced HF disease, which can be aging-related, hypoxia in AD results in reduction of ATP production, impaired mitochondrial function, increased ROS production, neuronal injury and inflammation. Mitochondrial function may be improved by enhancing mitochondrial biogenesis through caloric restriction and exercise, as well as the administration of CCrP crosses the blood brain barrier. Resveratrol is a natural product known for its anti-ageing properties due to calorie restriction like effects. It prevents oxidative damage and decreases apoptosis and cell injury. Resveratrol’s take on other neurological disorders is due to its anti-oxidative, anti-apoptotic, anti-inflammatory and cognitive and motor enhancement properties. It decreases oxidative stress and inflammation as noted by the decrease in inflammatory cytokines such as TNF-α, IL-6 and 1L-1β.

AD is a pressing public health problem with no effective treatment. Existing therapies only provide symptomatic relief without being able to prevent, stop or reverse the pathologic process. While the molecular basis underlying this multifactorial neurodegenerative disorder remains a significant challenge, mitochondrial dysfunction appears to be a critical factor in the pathogenesis of this disease. It is therefore important to target mitochondrial dysfunction in the prodromal phase of AD to slow or prevent the neurodegenerative process and restore neuronal function. Studies reported mechanisms of action and translational potential of current mitochondrial and bioenergetic therapeutics for AD including: mitochondrial enhancers to potentiate energy production; antioxidants to scavenge reactive oxygen species and reduce oxidative damage: glucose metabolism and substrate supply: and candidates that target apoptotic and mitophagy pathways to remove damaged mitochondria. While mitochondrial therapeutic strategies have shown promise at the preclinical stage, there has been little progress in clinical trials thus far. Current FDA-approved drugs for AD treatment include: N-methyl-D-aspartic acid (NMDA) receptor antagonist memantine and cholinesterase inhibitors donepezil, galantamine, and rivastigmine. These drugs augment cholinergic neurotransmission or attenuate excitotoxic neuronal injury . However, they only provide palliative benefits at best, with limited impact on the underlying disease mechanisms. Therefore, there is an urgent need for interventions that not only impact the aging process in favor of sustained brain health, but also promote successful brain aging in the context of neurodegenerative diseases.

Since heart and brain require the greatest amounts of energy, they are the most affected during failures of the mitochondria to generate ATP due to hypoperfusion, mitochondrial dysfunction is a key to aging and aging-related disease including cardiovascular diseases and neurodegenerative diseases such as Alzheimer’s disease. There is a link between the energy status of the cell and impaired organ function. Reduction of ATP production and the increase of oxidative stress are major triggers of neurons, and cardiac myocytes dysfunction, thereby contributing to not only “disease development,” but also progression of age-related disorders. The progression of HF is associated with diminished energy metabolism and a decrease in ATP synthesis capacity and a decrease in overall ATP levels. Age-related changes in mitochondria are associated with decline in mitochondrial function and ATP production. Aging is characterized by a general decrease in O₂ supply to tissues and a reduction in tissue pO₂. A diminished vascularization (lack of blood flow) in aging alters the diffusion of O₂ at the capillary tissue level, and at an advanced stage, this can lead to tissue hypoxia.

Autophagy is an intracellular self-digesting pathway to remove abnormal organelles, malformed proteins, and surplus or unnecessary cytoplasmic contents through lysosomal digestion. It is the main lysosomal degradative machinery, plays a major role in maintaining cellular homeostasis and, thus, a healthy state in an organism. This process recycles unnecessary or damaged substances, therefore, not only providing nutrients to maintain vital cellular functions in times of starvation but also eliminating potentially harmful cellular materials. Importantly, the autophagic rate declines with increasing age, suggesting a functional correlation between aging and autophagy. Indeed, the deregulation of autophagy is involved in the onset of various age-related diseases such as cancer, cardiomyopathy, type II diabetes, and neurodegeneration. Early studies on rat hepatocytes suggested that the execution of autophagy depends on energy availability since inhibition of ATP production stalls autophagic flux. Until recently, aging was regarded as an unregulated and inescapable consequence of the accumulation of incidental damage in macromolecules and/or organelles. However, the discovery of multiple ways to extend the lifespan in a variety of different model organisms, e.g., by genetic and pharmacological means, developed the formulation of alternative aging theories that consider aging as a molecular program. Therefore, there is a need to develop future therapeutic interventions to improve energy supply with the goal of improving the quality of life in the elderly and reduce the development and progression of age-related diseases such as HF and Alzheimer.

In the present invention, CCrP is a mitochondria-targeted protective compound which prevents mitochondrial dysfunction and constitutes a potential new therapeutic strategy in the prevention and treatment of ischemic and aging-related involving ischemia-induced injury cardiovascular and central nervous system diseases including but not limited, to HF, Alzheimer’s disease, stroke, ocular ischemia-induced injury and diseases, optic nerve ischemia-induced injury and diseases, and retinal ischemia-induced injury and diseases.

In the present invention, CCrP can also function as anti-aging drug during the aging process due to its ability to preserve mitochondrial function and increase ATP production, thus, decreases apoptosis and inflammation, resulting in restoration of cognitive and motor function.

CCrP is a novel mechanism for preventing development of heart failure. The bioenergetic CCrP is a promising first-in-class cardioprotective drug that prevents the development of heart failure due to ischemia. Thus, preservation of ATP by CCrP treatment prevents ischemic injury, reduces disease progression and restores organ function. In addition to HF as an example of cardiovascular disease, similarly, CCrP will slow down the aging process resulting in organ rejuvenation in neuro diseases characterized as ischemia-induced and/or aging-related diseases including, Alzheimer’s disease, ocular ischemia-induced injury and diseases, optic nerve ischemia-induced injury and diseases, and retinal ischemia-induced injury and diseases, and stroke.

Accordingly, an embodiment of the present disclosure provides a method for preventative and therapeutic treatment of neuro ischemic diseases and disorders involving ischemia-induced injury, including aging-related disorders, the method comprising the steps of: (a) recruiting a subject; (b) monitoring the subject for presence of ischemic events, ischemia-induced injury, and tissue deterioration by assessing the levels of external markers for the ischemic events, ischemia-induced injury, and tissue deterioration known for characterizing neuro ischemic diseases and disorders involving ischemia-induced injury; (c) collecting a first set of samples from the subject; (d) analyzing the first set of samples and assessing for the expression and release of neutrophil chemotactic factor referred to as Nourin protein levels, for ATP levels, and for the levels of internal markers of the downstream determiners for the ischemic events, ischemia-induced injury, and tissue deterioration selected from inflammation, apoptosis, necrosis, organ failure, and loss of organ function markers known for characterizing neuro ischemic diseases and disorders involving ischemia-induced injury; (e) classifying the monitored subject in terms of the levels of said external markers of step (b), and the levels of said internal markers of step (d) to determine the stage and progress of the ischemic events, ischemia-induced injury, and tissue deterioration in the subject to understand the severity of the neuro ischemic diseases and disorders involving ischemia-induced injury and to calculate a therapeutically effective amount of a bioenergetic agent to be administered to the subject; (f) administrating to the subject the therapeutically effective amount of the bioenergetic agent as calculated in step (e); (g) monitoring the subject again at various time-intervals after said administrating of the bioenergetic agent by assessing the levels of external markers for the ischemic events, ischemia-induced injury, and tissue deterioration known for characterizing neuro ischemic diseases and disorders involving ischemia-induced injury; (h) collecting from the subject a second set of samples after administrating the bioenergetic agent and subsequent sets of samples at various time-intervals after said administrating: (i) analyzing the second set of samples and the subsequent sets of samples of step (h) and assessing for the expression and release of neutrophil chemotactic factor referred to as Nourin protein levels, for ATP levels, and for the levels of internal markers of the downstream determiners for the ischemic events, ischemia-induced injury, and tissue deterioration selected from inflammation, apoptosis, necrosis, organ failure, and loss of organ function markers known for characterizing neuro ischemic diseases and disorders involving ischemia-induced injury; (j) calculating the effectiveness of the bioenergetic agent in terms of the expression and release of neutrophil chemotactic factor referred to as Nourin protein, and the ATP levels by comparing their levels in the first set of samples as analyzed in step (d) to the second set of samples and subsequent sets of samples after said administrating as analyzed in step (i); (k) calculating the presence, progress, and stage of the ischemic events, ischemia-induced injury, and tissue deterioration after said administrating of the bioenergetic agent in terms of the levels of external markers for the ischemic events, ischemia-induced injury, and tissue deterioration as assessed in step (b) in comparison to as assessed after said administrating in step (g), and the levels of internal markers of the downstream determiners for the ischemic events, ischemia-induced injury, and tissue deterioration selected from inflammation, apoptosis, necrosis, organ failure, and loss of organ function markers by comparing their levels in the first set of samples as analyzed in step (d) to the second set of samples and subsequent sets of samples after said administrating as analyzed in step (i) to check the effectivity of the bioenergetic agent in halting or reversing progress, prevention, or treatment of neuro ischemic diseases and disorders involving ischemia-induced injury, wherein the bioenergetic agent is a synthetic analogue that maintains and restores mitochondrial bioenergetics associated with ATP generation disrupted in ischemic events and ischemia-induced injury associated with neuro ischemic diseases and disorders involving ischemia-induced injury, wherein the ischemia-induced injury comprises a tissue in an organ of the subject exposed to injury, hypoxia, or ischemia associated with neuro ischemic diseases and disorders involving ischemia-induced injury, and wherein the ischemia comprises warm, cold, or demand ischemia, wherein the bioenergetic agent preserves mitochondrial biogenesis, prevents cell injury, prevents disease development, rejuvenates organ function, and restores normal physical activity, and wherein the bioenergetic agent is selected from 1-carboxymethyl-2-iminoimidazolidine referred to as Cyclocreatine (CCr), and 1-carboxymethyl-2-imino-3-phosphonoimidazolidine referred to as Cyclocreatine phosphate (CCrP).

In another embodiment of the method for preventative and therapeutic treatment of neuro ischemic diseases and disorders involving ischemia-induced injury as disclosed herein, wherein the therapeutically effective amount of a bioenergetic agent given to the subject comprises administrating an amount of the bioenergetic agent in a range between 0.3 g per kg per day and 1.5 g per kg per day of body weight as calculated for administrating the bioenergetic agent once daily or more than once daily for a cumulative amount administered to be in a range of between 0.3 g per kg per day and 1.5 g per kg per day of body weight based on step (e) as disclosed hereinabove.

In another embodiment of the method for preventative and therapeutic treatment of neuro ischemic diseases and disorders involving ischemia-induced injury as disclosed herein, wherein the bioenergetic agent is administered daily at an amount as calculated in step (e) as disclosed hereinabove such that there is no change in heart, liver, brain, and kidney function which is indicative of absence of organ toxicity.

In another embodiment of the method for preventative and therapeutic treatment of neuro ischemic diseases and disorders involving ischemia-induced injury as disclosed herein, wherein the administrating of the bioenergetic agent is selected from a group consisting of: prophylactic administration of the bioenergetic agent by injection daily, in a range of time between 7 days and 10 minutes prior to, or by injection immediately prior to injury or in a range of time between a few hours and 30 days post injury to protect against ischemic damage, and to prevent, treat, inhibit, or reduce tissue deterioration and disease progression associated with the downstream determiners selected from inflammation, apoptosis, necrosis, organ failure, and loss of organ function, for prevention of neuro ischemic diseases and disorders involving ischemia-induced injury, and therapeutic administration of the bioenergetic agent during injury or immediately post-injury for few hours, then daily for 1, 7 to 14 days, weeks, months and years to treat ischemic disease, to prevent and slowdown tissue deterioration, to inhibit or reduce ischemic disease progression associated with the downstream determiners selected from inflammation, apoptosis, necrosis, organ failure, and loss of organ function, and to stop or reverse the pathologic process for treatment of neuro ischemic diseases and disorders involving ischemia-induced injury.

In another embodiment of the method for preventative and therapeutic treatment of neuro ischemic diseases and disorders involving ischemia-induced injury as disclosed herein, wherein the bioenergetic agent 1-carboxymethyl-2-iminoimidazolidine referred to as Cyclocreatine (CCr) is insoluble form of the bioenergetic agent, and 1-carboxymethyl-2-imino-3-phosphonoimidazolidine referred to as Cyclocreatine phosphate (CCrP) is soluble form of the bioenergetic agent, and wherein the bioenergetic agent is administered in a form, in a solvent, and in solution so as to cross the blood-brain barrier to be effective in prevention and treatment of neuro ischemic diseases and disorders involving ischemia-induced injury. The soluble form, CCrP is prepared with 60% weight by weight (w/w) of CCr in the CCrP preparation and 40% weight by weight (w/w) of the phosphorous moiety. CCrP in vivo quickly undergoes phosphorous dephosphorylation to lipophilic CCr form which easily crosses the blood brain barrier. Within the tissue, CCr is rephospharylated and stored until a hypoxic/ischemic episode. In another embodiment of the method for preventative and therapeutic treatment of neuro ischemic diseases and disorders involving ischemia-induced injury as disclosed herein, wherein the bioenergetic agent is administered by a route of administration selected from a group of routes of administration comprising, intravenously, intraperitoneally, orally, intranasally, subcutaneously, intrathecally, intraventricularly, and intramuscularly.

In another embodiment of the method for preventative and therapeutic treatment of neuro ischemic diseases and disorders involving ischemia-induced injury as disclosed herein, wherein the subject is a human being or an animal, wherein the subject is a healthy subject or a diseased subject, and wherein the diseased subject is characterized and classified as suffering from neuro ischemic diseases and disorders involving ischemia-induced injury in terms of the levels of external markers for the ischemic events, ischemia-induced injury, and tissue deterioration, and the levels of internal markers of the downstream determiners for the ischemic events, ischemia-induced injury, and tissue deterioration selected from inflammation, apoptosis, necrosis, organ failure, and loss of organ function markers as compared to known standard levels of said external markers and said internal markers in healthy subjects.

In another embodiment of the method for preventative and therapeutic treatment of neuro ischemic diseases and disorders involving ischemia-induced injury as disclosed herein, wherein the external markers for the ischemic events, ischemia-induced injury, and tissue deterioration known for characterizing neuro ischemic diseases and disorders involving ischemia-induced injury comprise motor and sensory skills, balance and coordination, mental status which assesses the subject’s level of awareness and interaction with the environment, reflexes, cognitive screening tests, and functioning of the nerves, and wherein said external markers are assessed depending on factors, including the initial problem that the subject is experiencing or presenting with, the age of the subject, and the condition of the subject.

In another embodiment of the method for preventative and therapeutic treatment of neuro ischemic diseases and disorders involving ischemia-induced injury as disclosed herein, wherein the internal markers of the downstream determiners for the ischemic events, ischemia-induced injury, and tissue deterioration selected from inflammation, apoptosis, necrosis, organ failure, and loss of organ function markers known for characterizing neuro ischemic diseases and disorders involving ischemia-induced injury comprise inflammatory and anti-inflammatory markers comprising cytokine and chemokine levels, apoptosis and anti-apoptosis markers, necrosis and anti-necrosis markers, markers for organ failure of organs including brain, liver, heart, kidney, vasculature, and markers for loss of organ function of organs including brain, liver, heart, kidney, vasculature. Examples of the said internal markers include TNF-α, TGF-β2, TGF-β3, IL-6, IL-8, IL-2, IL-1β, TLR1 to TLR9, HIF-α, adhesion molecules such as LECAM-1, ICAM-1, ELAM-1, collagenase type IV, N-acetyl-B-glucosaminidase, gelatinases, superoxide anion, BNP, caspase-3, eNOS, and hs-cTnI.

In another embodiment of the method for preventative and therapeutic treatment of neuro ischemic diseases and disorders involving ischemia-induced injury as disclosed herein, wherein the neuro ischemic diseases and disorders involving ischemia-induced injury comprise a group selected from Alzheimer’s disease, hypoxia/ischemia-induced stroke, ocular ischemia-induced injury and diseases, optic nerve ischemia-induced injury and diseases, and retinal ischemia-induced injury and diseases. The aforementioned disorders include aging-related stroke.

An embodiment of the present disclosure provides a method for prevention and treatment of ischemic events and ischemia-induced injury and tissue deterioration associated with neurologic surgical procedures, the method comprising the steps of: (i) recruiting a subject set to undergo a neurologic surgical procedure; (ii) monitoring the subject for presence of ischemic events, ischemia-induced injury and tissue deterioration by assessing the levels of external markers for the presence of ischemic events, ischemia-induced injury and tissue deterioration known to be associated with neurologic surgical procedures; (iii) collecting a first set of samples from the subject before the performance of the neurologic surgical procedure that the subject is set to undergo; (iv) analyzing the first set of samples and assessing for the expression and release of neutrophil chemotactic factor referred to as Nourin protein levels, for ATP levels, and for the levels of internal markers of the downstream determiners for the ischemic events, ischemia-induced injury and tissue deterioration selected from inflammation, apoptosis, necrosis, organ failure, and loss of organ function markers known to be associated with neurologic surgical procedures; (v) classifying the monitored subject in terms of the levels of said external markers of step (ii), and the levels of said internal markers of step (iv) to determine the stage and progress of the ischemic events, ischemia-induced injury and tissue deterioration in the subject to understand the condition of the subject set to undergo the neurologic surgical procedure and to calculate a therapeutically effective amount of a bioenergetic agent to be administered to the subject; (vi) administrating to the subject the therapeutically effective amount of the bioenergetic agent as calculated in step (v); (vii) monitoring the subject again at various time-intervals after said administrating of the bioenergetic agent by assessing the levels of external markers for the ischemic events, ischemia-induced injury and tissue deterioration known to be associated with neurologic surgical procedures; (viii) collecting from the subject a second set of samples after administrating the bioenergetic agent and subsequent sets of samples at various time-intervals after said administrating; (ix) analyzing the second set of samples and the subsequent sets of samples of step (viii) and assessing for the expression and release of neutrophil chemotactic factor referred to as Nourin protein levels, for ATP levels, and for the levels of internal markers of the downstream determiners for the ischemic events, ischemia-induced injury and tissue deterioration selected from inflammation, apoptosis, necrosis, organ failure, and loss of organ function markers known to be associated with neurologic surgical procedures; (x) calculating the effectiveness of the bioenergetic agent in terms of the expression and release of neutrophil chemotactic factor referred to as Nourin protein, and the ATP levels by comparing their levels in the first set of samples as analyzed in step (iv) to the second set of samples and subsequent sets of samples after said administrating as analyzed in step (ix); (xi) calculating the presence, progress, and stage of the ischemic events, ischemia-induced injury and tissue deterioration after said administrating of the bioenergetic agent in terms of the levels of external markers as assessed in step (ii) in comparison to as assessed after said administrating in step (vii), and the levels of internal markers as analyzed in step (iv) in comparison to the as assessed after said administrating as analyzed in step (ix) to check the effectivity of the bioenergetic agent in halting or reversing progress, prevention, or treatment of ischemic events, ischemia-induced injury and tissue deterioration associated with neurologic surgical procedures, wherein the bioenergetic agent is a synthetic analogue that maintains and restores mitochondrial bioenergetics associated with ATP generation disrupted in ischemic events, ischemia-induced injury and tissue deterioration associated with neurologic surgical procedures, wherein the ischemia-induced injury comprises a tissue in an organ of the subject exposed to injury, hypoxia, or ischemia associated with neurologic surgical procedures involving ischemia-induced injury, and wherein the ischemia comprises warm, cold, or demand ischemia, wherein the bioenergetic agent preserves mitochondrial biogenesis, prevents cell injury, prevents disease development, rejuvenates organ function, and restores normal physical activity, and wherein the bioenergetic agent is selected from 1-carboxymethyl-2-iminoimidazolidine referred to as Cyclocreatine (CCr), and 1-carboxymethyl-2-imino-3-phosphonoimidazolidine referred to as Cyclocreatine phosphate (CCrP).

In another embodiment of the method for prevention and treatment of ischemic events and ischemia-induced injury and tissue deterioration associated with neurologic surgical procedures as disclosed herein, wherein wherein the therapeutically effective amount of a bioenergetic agent given to the subject comprises administrating an amount of the bioenergetic agent in a range between 0.3 g per kg per day and 1.5 g per kg per day of body weight as calculated for administrating the bioenergetic agent once daily or more than once daily for a cumulative amount administered to be in a range of between 0.3 g per kg per day and 1.5 g per kg per day of body weight based on step (v) as disclosed hereinabove.

In another embodiment of the method for prevention and treatment of ischemic events and ischemia-induced injury and tissue deterioration associated with neurologic surgical procedures as disclosed herein, wherein the bioenergetic agent is administered daily at an amount as calculated in step (v) as disclosed hereinabove such that there is no change in heart, liver, brain, and kidney function which is indicative of absence of organ toxicity.

In another embodiment of the method for prevention and treatment of ischemic events and ischemia-induced injury and tissue deterioration associated with neurologic surgical procedures as disclosed herein, wherein the administrating of the bioenergetic agent is selected from a group consisting of: prophylactic administration of the bioenergetic agent by injection daily, in a range of time between 7 days and 10 minutes prior to, or by injection immediately prior to when the subject is set to undergo a neurologic surgical procedure or in a range of time between a few hours and 30 days post the neurological procedure to protect against ischemic damage, and to prevent, treat, inhibit, or reduce tissue deterioration and disease progression associated with the downstream determiners selected from inflammation, apoptosis, necrosis, organ failure, and loss of organ function, for prevention of ischemic events, ischemia-induced injury and tissue deterioration known to be associated with neurologic surgical procedures, and therapeutic administration of the bioenergetic agent during the neurological procedure or immediately post the neurological procedure for few hours, then daily for 1, 7 to 14 days, weeks, months and years to treat ischemic disease, to prevent and slowdown tissue deterioration, to inhibit or reduce ischemic disease progression associated with the downstream determiners selected from inflammation, apoptosis, necrosis, organ failure, and loss of organ function, and to stop or reverse the pathologic process for treatment of ischemic events, ischemia-induced injury and tissue deterioration associated with neurologic surgical procedures.

In another embodiment of the method for prevention and treatment of ischemic events and ischemia-induced injury and tissue deterioration associated with neurologic surgical procedures as disclosed herein, wherein the bioenergetic agent 1-carboxymethyl-2-iminoimidazolidine referred to as Cyclocreatine (CCr) is insoluble form of the bioenergetic agent, and 1-carboxymethyl-2-imino-3-phosphonoimidazolidine referred to as Cyclocreatine phosphate (CCrP) is soluble form of the bioenergetic agent, and wherein the bioenergetic agent is administered in a form, in a solvent, and in solution so as to cross the blood-brain barrier to be effective in prevention and treatment of neuro ischemic diseases and disorders involving ischemia-induced injury. The soluble form, CCrP is prepared with 60% weight by weight (w/w) of CCr in the CCrP preparation and 40% weight by weight (w/w) of the phosphorous moiety. CCrP in vivo quickky undergoes phosphorous dephosphorylation to lipophilic CCr form which easily crosses the blood brain barrier. Within the tissue, CCr is rephospharylated and stored until a hypoxic/ischemic episode.

In another embodiment of the method for prevention and treatment of ischemic events and ischemia-induced injury and tissue deterioration associated with neurologic surgical procedures as disclosed herein, wherein the bioenergetic agent is administered by a route of administration selected from a group of routes of administration comprising, intravenously, intraperitoneally, orally, intranasally, subcutaneously, intrathecally, intraventricularly, and intramuscularly.

In another embodiment of the method for prevention and treatment of ischemic events and ischemia-induced injury and tissue deterioration associated with neurologic surgical procedures as disclosed herein, wherein the subject is a human being or an animal.

In another embodiment of the method for prevention and treatment of ischemic events and ischemia-induced injury and tissue deterioration associated with neurologic surgical procedures as disclosed herein, wherein the external markers for the presence of ischemic events, ischemia-induced injury and tissue deterioration known to be associated with neurologic surgical procedures comprise motor and sensory skills, balance and coordination, mental status which assesses the subject’s level of awareness and interaction with the environment, reflexes, cognitive screening tests, and functioning of the nerves, and wherein said external markers are assessed depending on factors, including the initial problem that the subject is experiencing or presenting with, the age of the subject, and the condition of the subject.

In another embodiment of the method for prevention and treatment of ischemic events and ischemia-induced injury and tissue deterioration associated with neurologic surgical procedures as disclosed herein, wherein the internal markers of the downstream determiners for the presence of ischemic events, ischemia-induced injury and tissue deterioration selected from inflammation, apoptosis, necrosis, organ failure, and loss of organ function markers known to be associated with neurologic surgical procedures comprise inflammatory and anti-inflammatory markers comprising cytokine and chemokine levels, apoptosis and anti-apoptosis markers, necrosis and anti-necrosis markers, markers for organ failure of organs including brain, liver, heart, kidney, vasculature, and markers for loss of organ function of organs including brain, liver, heart, kidney, vasculature. Examples of the said internal markers include TNF-α, TGF-β2. TGF-β3. IL-6, IL-8, IL-2, IL-1β, TLR1 to TLR9, HIF-α, adhesion molecules such as LECAM-1, ICAM-1. ELAM-1, collagenase type IV, N-acetyl-B-glucosaminidase, gelatinases, superoxide anion, BNP, caspase-3, eNOS, and hs-cTnI.

In another embodiment of the method for prevention and treatment of ischemic events and ischemia-induced injury and tissue deterioration associated with neurologic surgical procedures as disclosed herein, wherein the neurologic surgical procedures comprise intracerebral hemorrhage surgery, arterial repair, ocular surgery, optic nerve surgery, retinal surgery, non-nerve-related surgery that is capable of causing ischemia of the nervous system and other brain surgery to protect against ischemic damage, and to prevent, inhibit or reduce tissue deterioration and disease progression.

The invention will be further explained by the following Examples, which are intended to purely exemplary of the invention, and should not be considered as limiting the invention in any way.

### EXAMPLES

### Example 1 - Cyclocreatine (CCr) and Cyclocreatine Phosphate (CCrP) Preserve ATP Production During Ischemia.

Mitochondrial dysfunction and reduction of ATP production are known to play an important role in ischemic heart diseases. A critical mechanism of how hypoxia/ischemia causes irreversible myocardial injury is through the exhaustion of ATP. Depletion of ATP during ischemia is one of the major factors in tissue apoptosis and inflammation. Contractile performance decreases precipitously and ceases when only 20% of ATP is depleted. To date, there are no clinical options available that directly address preservation of ATP during ischemia and reperfusion . Thus, CCrP is a new pharmacologic agent - that has the ability to maintain and restore myocardial energetics in the setting of ischemia and reperfusion: thus would address a very important unmet need in the clinical care of patients with myocardial ischemia and necrosis.

Creatine (Cr) is the naturally occurring compound necessary for myocardial contractility. Cyclocreatine (CCr) is a synthetic analogue of Cr and it acts as a potent bioenergetic protective agent by preserving high levels of ATP in ischemic myocardium. In the heart, Cr and CCr are converted to CrP and CCrP, respectively by the mitochondrial Creatine Kinase enzyme. When CCr is administered to animals before ischemia, it gets stored in myocardial tissue as CCrP, while when CCrP is administered intravenously, it loses its phosphorous group in circulation and becomes CCr. In the heart, CCr is converted to CCrP and stored in the myocardium until an ischemic event, in which it will generate ATP by phosphorylating adenosine diphosphate (ADP). During ischemia, the generation of ATP is through the CrP system (i.e., mitochondrial Creatine Kinase enzyme) as well as, glycolysis where the heart alternatively shifts to anaerobic glycolysis for its requisite energy production. Unfortunately, glycolysis is quite inefficient because ischemic heart catabolizes glucose and produces lactic acid. The generated tissue acidity results in a quick reduction of CrP function. On the other hand, CCrP is much more stable and is a superior long-acting phosphagen than CrP as it sustains ATP synthesis longer during ischemia by continuing phosphorylating ADP at low acidity. Thus, CCrP has the ability to sustain high levels of ATP during ischemia.

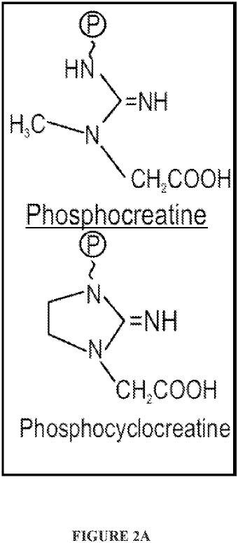

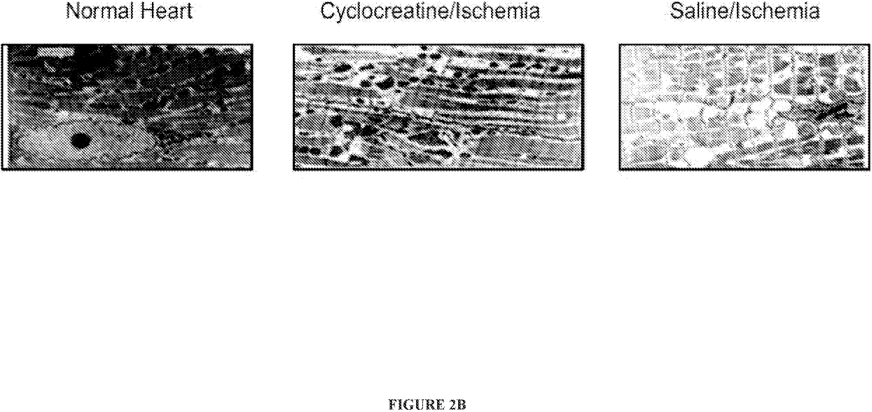

Cyclocreatine crosses the blood-brain barrier. In the Cyclocreatine Phosphate (CCrP) preparation, CCr represents only 40% of CCrP (FIG. 2A). CCrP is a new class of therapy which preserves cellular energy and prevents ischemic Injury. It is a first-in-class therapy which works directly on myocardial cells to prevent ischemic injury and protects again tissue deterioration. It works by preserving cellular ATP during ischemia and thus interfere with and reverse the ischemic pathology. It has been demonstrated that the significant cardioprotection of Nourexal in animal models of myocardial infraction, cardiopulmonary bypass surgery, cardiac arrest, and heart transplantation. Specifically, the FDA has recently awarded Nour Heart the Orphan Drug Status for Nourexal with the unique designation for the “Prevention of Ischemic Injury to Enhance Cardiac Graft Recovery and Survival in Heart Transplantation” (DRU-2015-4951).

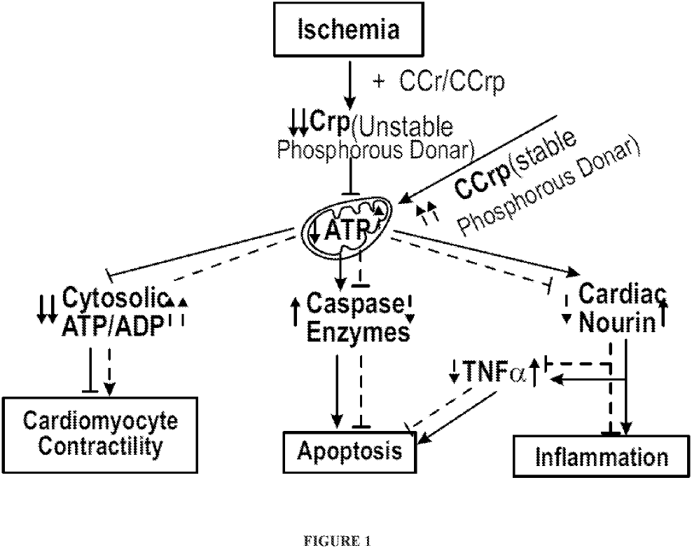

FIG. 1 is a proposed mechanism of action of the cardioprotective effect of Cyclocreatine (CCr) and Cyclocreatine Phosphate (CCrP). Myocardial ischemia is a major denominator of many cardiac diseases, including: CAD. UA, AMI and HF. Myocardial apoptosis and inflammation are the hallmarks of the tissue response to ischemia/ reperfusion injury. Depletion of ATP during ischemia is one of the major factors that accelerates the apoptotic process of healthy myocardial tissue and triggers inflammation. Our proposed mechanism of action of the cardioprotective benefits of CCr and CCrP is through the preservation of high levels of myocardial ATP during ischemia and reduction of tissue injury and circulating Nourin. During reperfusion, CCr and CCrP will also reduce tissue inflammation, apoptosis, and edema resulting in immediate restoration of post-ischemic contractile function, without arrhythmias.

### Example 2 - Cyclocreatine and Cyclocreatine Phosphate Prevent Myocardial Ischemic Injury and Restore Contractile Function.

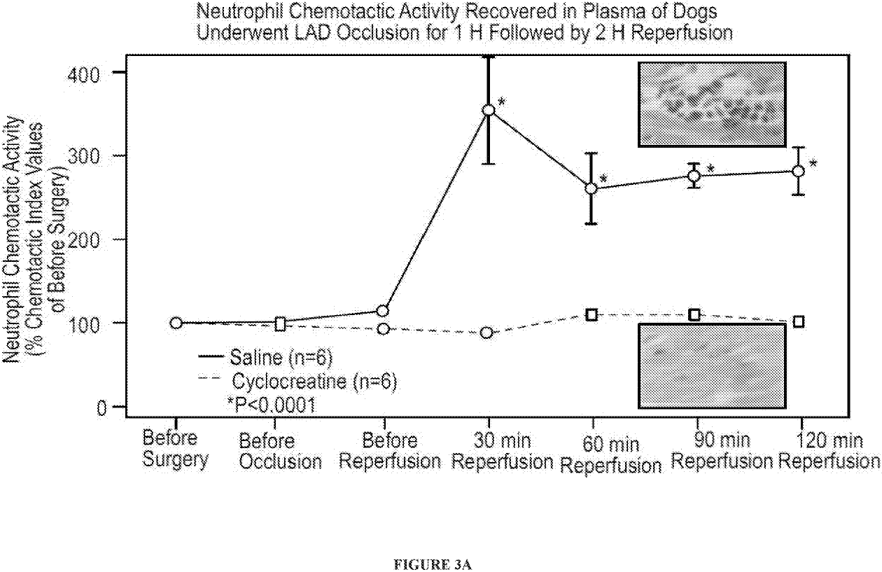

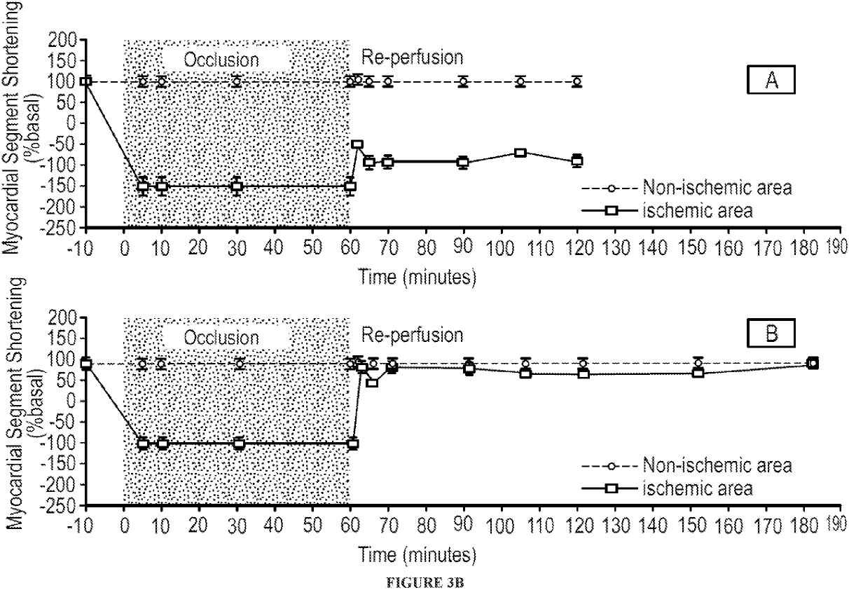

During ischemia, myocardial ATP levels decrease by 65% at 15 minutes and by 90% at 40 minutes, thus contractile performance in vivo decreases precipitously and ceases when only 20% of ATP and 75% of creatine phosphate (CrP) are depleted. It has been demonstrated that when CCr was administered to dogs 60 minutes before the induction of myocardial ischemia by occluding the Left Anterior Descending (LAD) coronary artery for 1 hour. ATP synthesis continued during ischemia and its depletion was delayed resulting in over 85% preservation of of pre-ischemic ATP level with a loss of only 15%, and 97% preservation of the CrP (loss of 3%) during ischemia. This significant preservation is crucial since ATP depletion of more than 20% ceases contractility. Control saline-treated hearts maintained only 66% of the ATP, with a loss of 34%, resulting in loss of reperfusion cardiac contractility. Histologically, the CCr-treated hearts showed markedly less myocardial cell injury when compared to the control (saline) group (FIG. 2B). As indicated in FIG. 3B, Cyclocreatine treatment restored over 80% of contractile function immediately at reperfusion and for an additional 2 hours. On the other hand, contractile function of control saline dogs was ceased completely after LAD occlusion and never recovered during reperfusion. Cyclocreatine also exhibited anti-inflammatory activity by inhibiting the levels of circulating Nourin protein in plasma samples collected during the 2 hour reperfusion, as well as reduces neutrophil accumulation into the myocardium at the end of 2 hours (FIG. 3A). The aforementioned results in FIG. 3 provide evidence for the expression of Nourin protein in response to vascular injury.

Clinically, Myocarditis is a condition where there is inflammation of the heart muscle. Inflammation of the heart muscle limits the heart’s ability to pump and can cause heart failure due to cardiac arrest or dilated cardiomyopathy. Therefore, “early” diagnosis of cardiac inflammation is key to preventing long-term heart damage. Viral infection is the most common cause of Myocarditis. Other causes are side effects of medications, autoimmune disorders, toxins and bacterial infections. Currently, Myocarditis can be diagnosed with the help of: (a) an electrocardiogram, echocardiogram, magnetic resonance imaging (MRI) to detect signs of inflammation of the heart muscle, heart enlargement and poor pumping function and abnormal rhythms the heart; (b) invasive cardiac catheterization and endomyocardial biopsy to check for cardiac inflammation in heart biopsies; and (c) blood tests to measure Troponin levels.

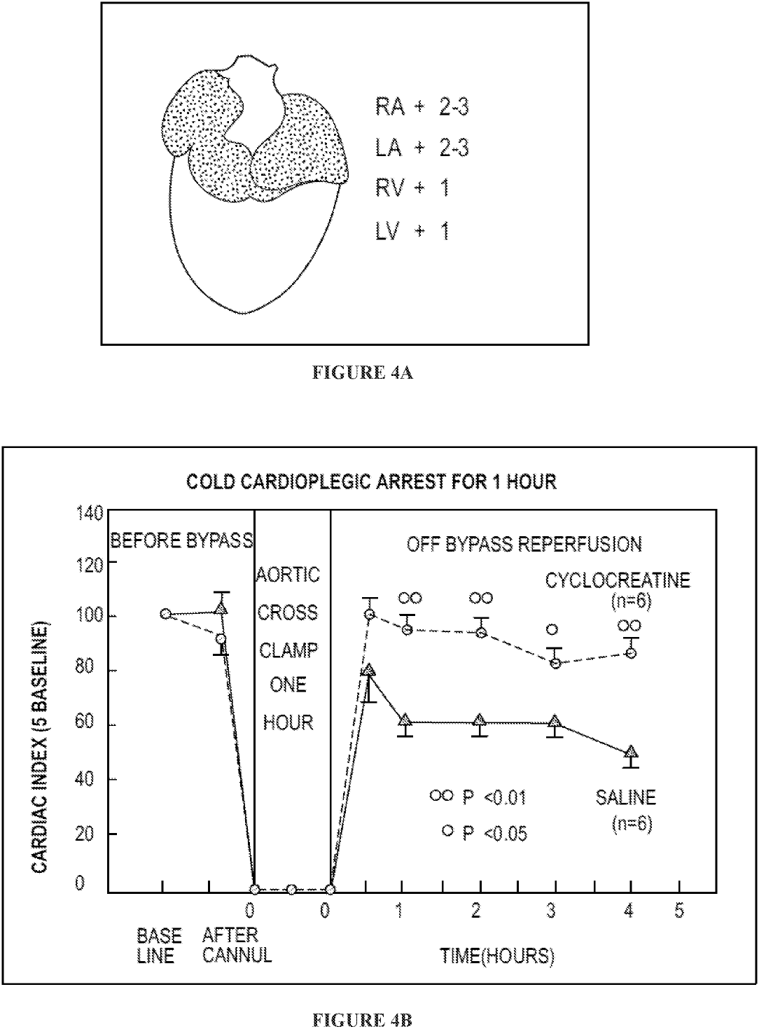

The present invention provides that the Nourin protein and its regulatory molecular network can be used as a non-invasive laboratory test for early diagnosis of cardiac inflammation. As indicated in FIG. 3A and FIG. 4A, high level of the inflammatory mediator, Nourin was released in response to myocardial ischemic injury and was associated with large accumulation of inflammatory cells during early reperfusion. It has also been previously reported that Nourin was released in response to viral infection induced by influenza flu infection both in-vitro and in-vivo. Nourin was detected in serum samples collected as early as 6 hours of mice inoculated with Swine Flu H1N1 virus and was associated with the development of severe lung inflammation. The administration of the Nourin competitive inhibitor, Cyclosporin H significantly reduced lung inflammation and inhibited Nourin activity in-vitro . Additionally, high level of Nourin was detected in plasma samples collected from patients with moderate to severe influenza flu infection compared to mild infection. Furthermore, it was demonstrated that Nourin is released by human vascular endothelial cells treated with the bacterial product, endotoxin. These studies suggest that the release of Nourin in response to viral and bacterial infection is associated with tissue inflammation.

In an intact canine model of cold cardioplegic arrest and aortic cross-clamping for 1 hour followed by reperfusion on bypass for 45 min and then off bypass for 4 hours, much higher neutrophil accumulation after reperfusion was observed in the right and left atria (+2-3) compared to the right and left ventriculars (+1) (FIG. 4A). As shown in FIG. 4B. post-bypass cardiac output was significantly better in CCr-treated hearts compared to that of controls, where the CCr-treated hearts achieved over 90% of the baseline function throughout the 4 hours of reperfusion, while control hearts achieved only 60% of the baseline function. All CCr-treated hearts restored contractile function immediately post-ischemic without arrhythmias, while, all control hearts required defibrillation. Atrial fibrillation (AF) induces cardiac structural remodeling and there is a need to develop more mechanism-directed AF therapies that use the mitochondria as a novel potential therapeutic target in AF. Our studies suggest the use of Cyclocreatine as a new anti-arrhythmic drug to preserve mitichondrial function and prevent inflammation-induced atrial fibrillation.

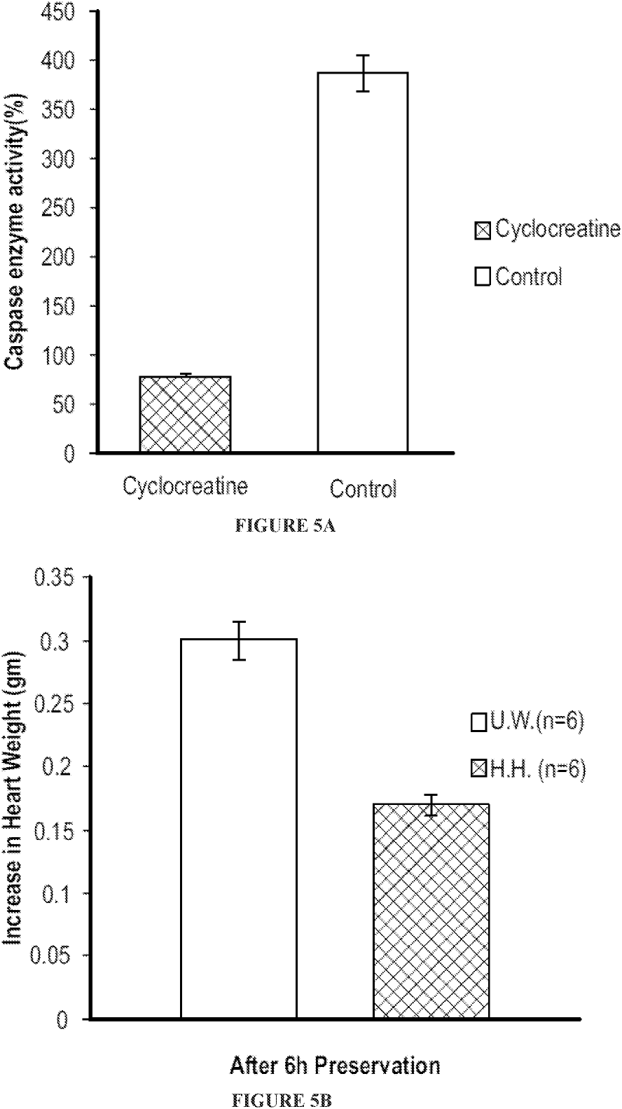

FIG. 5A indicates that Cyclocreatine is an anti-apoptotic agent by reducing apoptotic enzyme activity in the non-heartbeating dog model of heart transplantation. Dog hearts underwent 1 hour of global warm ischemic arrest then hearts were explanted and perfused ex vivo for an additional 4 hours with a cold lactated ringers solution containing Cyclocreatine, while control hearts received cold lactated ringers solution alone. Results indicated that exsanguination to induce global warm ischemia, the heart of the CCr-treated dog took 9 minutes to stop beating and develop asystole, while, control hearts completely stopped beating after an average of only 2 minutes. Similarly, the myocardium of the CCr dog maintained a tissue pH of 7.04±0.1 during the warm ischemia period of 1 hour and throughout the ex vivo perfusion interval, which was close to its baseline level of 7.11. On the other hand, tissue pH in control hearts fell to a nadir of 6.00±0.25 during the induction of warm ischemia and never returned back to baseline levels during the ex vivo preservation period. Furthermore, when compared to controls, CCr treatment demonstrated:
1) Three-fold increase of myocardial ATP content compared to controls,
2) Reduced intracellular edema compared to control as measured by diffusion weighted imaging on MRI,
3) Reduced myocardial tissue lactic acidosis compared to control as measured by spectroscopic imaging on MRI,
4) Reduced level of the cell injury marker Malondialdehyde compared to controls,
5) Significant reduction in apoptosis in CCr heart compared to controls as measured by Caspase enzyme activity.

FIG. 5A describes the reduction of the Caspase enzyme activities in the CCr group (25% reduction of baseline) compared to the significant stimulation observed in control dogs (3.86-fold increase over baseline). Interestingly, the significant reduction of Caspase activities in the CCr group indicates that the enzymes are present more in the “inactive proenzyme” forms.

Cyclocreatine Phosphate (CCrP) also reduced heart weight after 6 hours of cold storage in HH Solution (UW + CCrP) Compared to Control (UW) (FIG. 5B). Results indicated that the recovery of contractile function was significantly better in the CCrP treated-group (HH) compared to saline control. Furthermore, there was a higher weight gain in control hearts (UW) after 6 hours of cold storage compared to the CCrP-treated hearts (HH). As indicated in FIG. 5B, CCrP-treated hearts (HH) weighted only 0.25 gm while control hearts (UW) weighed 0.31 gm. The observed reduction of heart edema in the CCrP hearts (HH) is crucial for the restoration of contractile function during reperfusion at the end of 6-hour storage.

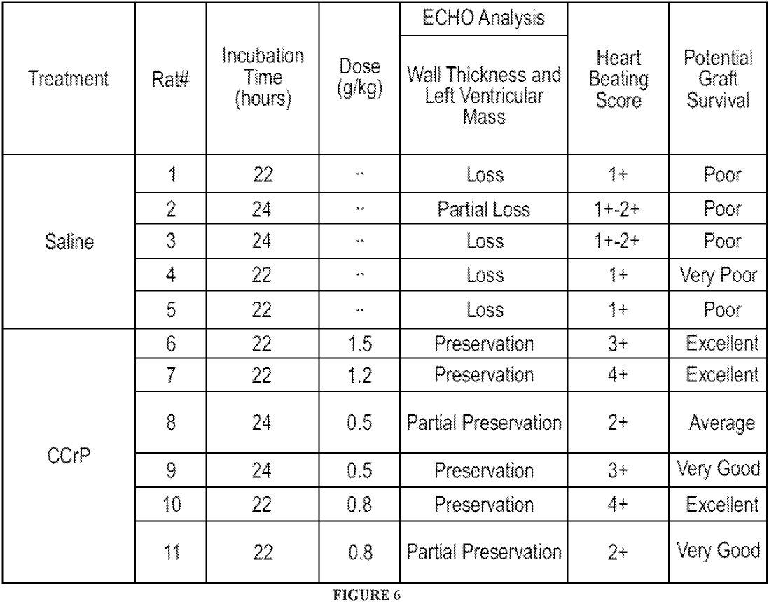

As indicated in FIG. 6, it was further demonstrated that Cyclocreatine Phosphate at 0.8 g/kg protected rat grafted hearts against ischemic injury during harvesting and prolonged cold storage for 22 Hours and 24 hours in the in vivo rat syngeneic abdominal heterotopic heart transplantation for 3 days. The CCrP treatment increased the survival of the grafted hearts in recipient rats for 3 days. Lewis rats were used for both the donors and recipients to avoid immunologic rejection. This approach allowed a focus at determining the in vivo cardioprotective benefits of CCrP treatment and to evaluate whether CCrP would restore cardiac contractility after prolonged cold storage and increase graft survival. Echocardiography (ECHO) analyses were conducted 2 hours after transplantation at day 0 and at day 3 before sacrifice.

CCrP treatment showed significant cardioprotection against early reperfusion injury after transplantation as illustrated by the absence of delayed heart function in the first 1 minute and the restoration of strong contractile function in all CCrP-treated hearts minutes after transplantation. In the contrary, saline-treated control donor grafts showed a slow start of heart beating with weaker contractile function. CCrP-treated hearts at doses of 0.8 gm/kg, 1.2 gm/kg and 1.5 gm/kg showed strong beating scores of 4+ and 3+ respectively at both day 0 and day 3 (score of +4 is the highest). However, the low dose of 0.5 gm/kg. showed strong heart beating scores of 4+ and 3+ right after transplantation at day 0 but partial myocardial protection by day 3 with beating scores ranged from 2+ to 3+. Saline-treated control donor rats were evidently dilated with an increase in the sizes of both ventricles and atria. Additionally, the color was mildly cyanotic and the contractility was poor and irregular in rhythm. In most control grafted hearts, their heart beating score ranged from 1+ to 2+ at day 0 and day 3. Protection was shown in most of CCrP grafted hearts at day 3 where the myocardial color and the consistency of the degree of contractility were almost the same as day zero. Additionally, the day 3 ECHO showed the continued preservation of the myocardial wall thickness and mass which are the main criteria that determine the degree of myocardial ischemia over a period of time. Most the control grafted hearts, on the other hand, continued to show evidence of ischemia as well as loss of wall thickness and cardiac mass by day 0 and day 3 (refer to FIG. 6).

In general, CCrP grafted hearts after 22 hours and 24 hours of incubation had good preservation of myocardial color and perfusion as well as contractile function as indicated by preservation of the myocardial wall thickness and mass compared to control saline grafted hearts. The general overall survival of the cardiac tissue of “CCrP-grafted hearts” was very good to excellent, while the general overall survival of the cardiac tissue of “control-grafted hearts” was poor. Based on these preclinical efficacy studies, the U.S. FDA has awarded the Orphan Drug Designation (ODD) status for CCrP for the: “Prevention of Ischemic Injury to Enhance Cardiac Graft Recovery and Survival in Heart Transplantation”.

### Example 3 - High Gene Expression of Nourin Gene-Based RNA Molecular Network (miR-137, miR-106b, mRNA-FTHL-17, mRNA-ANAPC11 and lncR-CTB89H12.4) as Biomarkers for Left Ventricular Remodeling After Myocardial Injury in Standard Isoproterenol (ISO) Rat Model of Heart Failure, and that Cyclocreatine Phosphate (CCrP) Treatment Prevented Ischemic Injury and Inhibited Gene Expression of the Nourin Gene-Based RNA Molecular Network in the Rat Heart Failure Model.

Mitochondrial abnormalities and reduced capacity to generate ATP can have a profound impact in HF. Abnormal mitochondria are also linked to myocyte injury because they are a major source of reactive oxygen species (ROS) production that can induce cellular damage. Isoproterenol (ISO) is a beta-adrenergic agonist which in high doses cause pathologic and molecular changes in rat heart that are similar to myocardial injury in humans. It causes coronary vasoconstriction, exaggerates myocardial Ca2+ influx and causes shunting of blood away from the subendocardial layer, producing subendocardial ischemia and cellular ATP depletion. ISO undergoes autoxidation, generating highly toxic ROS, which activate apoptotic pathways in the myocardium which in turn lead to contractile dysfunction and cardiomyocyte cell death. Therefore, high doses of ISO are used to induce experimental myocardial injury and to validate the effectiveness of test drug against ischemia-induced HF.

Cardiovascular disease is the leading cause of mortality in the United States and in westernized countries with ischemic heart disease accounting for the majority of these deaths. Myocardial infarction is the most common cause of heart failure. Virtually all episodes of ACS, including UA and both ST elevation myocardial infarction (STEMI - ischemic changes detected by Electrocardiogram (ECG) and non-ST elevation myocardial infarction (NSTEMI - no ischemic changes by ECG), are associated with the loss of myocardiocytes, edema, inflammation, fibrosis, and cardiac remodeling, which all together represent the leading pathophysiological mechanisms of HF. The immune system plays a significant role in ventricular remodeling, and its persistent activation may lead to long-term cardiac injury. As described in this invention, it was demonstrated that the high gene expression of Nourin molecular RNA Network in ACS patients, including UA. STEMI and NSTEMI, but not in healthy individuals.

The standard quantitative real time PCR (qPCR) molecular assay was used to determine the levels of Nourin RNA network in the ISO rat model of ischemia-induced HF. It was tested if the hypothesis that the levels of gene expression of Nourin molecular RNA Network (miR-137, miRNA-106b, mRNA FTHL-17, mRNA ANAPC11 and lncR-CTB89H12.4) will be elevated in HF rats, and that CCrP treatment will reduce their gene expression of Nourin molecular RNA Network in a dose-response manner.

Analysis of Nourin RNAs (lncR-CTB89H12.4, hsa-miR-106b, has-miR-137, mRNA-ANAPC11 and mRNA-FTHL-17) was performed on serum samples collected from 16 Healthy male volunteers with negative Troponin and negative treadmill stress test. Volunteers exercised on treadmill to confirm absence of ischemic heart disease. Samples were collected 30 minutes after the completion of the stress test. Females were excluded from this study because of the high false positive with the treadmill stress test procedure. Blood samples were obtained once within the first 8 hours of chest pain and were centrifuged and the serum was separated, aliquoted and stored immediately at -80° C. for further processing.

Blood samples were collected from 54 positive angina, 7 negative non-angina, 16 AMI patients and 16 healthy controls in primary blood collection tubes without clot activator and without anticoagulants such as EDTA or citrate (red-topped tubes). These blood samples were left at room temperature for a minimum of 30 min (and a maximum of 60 min) to allow complete blood clotting in the red-topped tubes. The clotted blood samples were then centrifuged at 1300xg at 4° C. for 20 min. The upper yellow serum was carefully removed, transferred to a polypropylene capped tube in 1 ml aliquots and stored at -80° C. until they are assayed by qPCR. All serum samples were labeled with a unique identifier to protect the confidentiality of the patients. None of the serum samples were allowed to thaw before analysis to minimize protein degradation and precipitation.

Biomarker validation using qPCR involved (1) extraction of the total miRNAs and total RNAs from serum samples (AMI and healthy); (2) synthesis of cDNA through reverse transcription; (3) measurement of cDNA using qPCR; and (4) evaluation of results by the plot curve analysis software of Rotor Gene to confirm specificities then amplification plot and data analysis. For the extraction of total RNA. including IncRNA, miRNA and mRNA from sera samples, miRNEasy RNA isolation kit (Qiagen, Hilden, Germany) was used according to manufacturer’s instructions. The RNA samples were dissolved in 14 µl of nuclease-free water. The concentration of RNA was determined using a NanoDrop spectrophotometer (Thermo Scientific. USA). Total cDNA including cDNA for miRNA, mRNAs and IncRNA was prepared from sera samples and were loaded to Thermal cycler instrument (Thermo Electron Waltham, MA) using miScript II RT Kit (Qiagen, Germany), and the reaction mix is composed of 2 ul 10× miScript Nucleics Mix, 4 ul 5x miScript HiFlex Buffer. 1 ul miScript Reverse Transcriptase Mix and RNase free water to 2 ug RNA and the mixture was incubated for 60 minutes at 37° C. then for 5 minutes at 95° C.

Quantification of the expression pattern and levels of Nourin gene-based RNA network panel by qPCR included: lncR-CTB89H12.4, mRNA FTHL-17 and mRNA ANAPC11. Expression in sera samples were quantified by adding 10 ul 2×RT²SYBR Green ROX qPCR Mastermix and QuantiTect SYBR Green PCR Kit, respectively, RT²IncRNAq PCR Assay for RT² IncRNA qPCR Assay for Human CSNK1A1 (ENST00000499521) (assay ID: LPH41640A). Hs_FTHL17_1_SG QuantiTect Primer Assay (NM__031894) (assay ID: QT00217966), Hs_ANAPC11 primer assay (assay ID: QT00243964), 2 ul template cDNA and RNase free water to a final volume of 20 ul Hs_ACTB_1_SG QuantiTect Primer Assay (NM_001101) (assay ID: QT00095431), was used as housekeeping gene to normalize our raw data as the invariant control for the samples, and compared with a reference sample. The PCR cycling program for relative lncR-CTB89H12.4 quantification was conducted as follow: firstly, denaturation at 95° C. for 10 min; followed by 45 cycles of denaturation for 15 seconds at 95° C.; then annealing for 30 seconds at 55° C. and extension for 30 seconds at 70° C.

To quantify the expression of hsa-miR-106b and hsa-miR-137. a miScript primer assays which target the hsa-miR-106b and hsa-miR-137 were purchased from Qiagen, Hilden. Germany. The primer assays ID and sequences are: hsa-miR-106b: miRNA: has-miR-106b-5p, assay ID: MIMAT0000680 with provided sequence “5′ UAAAGUGCUGACAGUGCAGAU” (SEQ ID NO:1) and for hsa-miR_137, assay ID: MIMAT0000429 and provided sequence “5′ UUAUUGCUUAAGAAUACGCGUAG” (SEQ ID NO:2). The RUN6 primer assay was used as a housekeeper gene for gene normalization.

For miRNAs amplification by quantitative Real time PCR (qPCR), miScript SYBR Green PCR Kit (Qiagen/SA Biosciences Corporation, Frederick, MD) was used. The 20 µl reaction mix is prepared by adding 10 ul 2x miScript SYBR Green PCR Master Mix, 2 ul 10× miScript Universal Primer. 2 ul 10× miScript Primer, 2 µl of template cDNA and 4 µl RNase free water, miR-106b and RUN6. The real-time cycler was programmed for relative quantification of hsa-miR-106b and Hsa-miR-137 as follows: initial activation step for 15 min at 95° C. to activate HotStarTaq DNA Polymerase. 40 cycle of PCR were performed under the following conditions; 15 seconds at 94° C., 30 seconds at 55° C. and 30 seconds at 72° C. for denaturation, annealing and extension respectively. Each reaction was carried out in triplicate. Relative quantification of RNA-based biomarker panel expression was calculated using Leviak method RQ= 2-ΔΔCt method. The threshold cycle (Ct) value of each sample was calculated using the Rotor Gene real time PCR detection system (Qiagen. Hilden. Germany). Any Ct value more than 36 was considered negative. The results were analyzed by the plot curve analysis software of Rotor Gene. Amplification plots and Tm values were analyzed to confirm the specificities of the amplicons for SybrGreen-base amplification.

For Nourin RNA’s stability in the collected blood samples, previously stored sera samples at -70 for about (3 to 4 months) were assayed. Sera samples were processed within half an hour after collection and aliquoted to minimize freeze thaw cycle. Spin columns with small pore sizes were used in an attempt to concentrate serum RNA before the precipitation step and have checked the concentration and purity of RNA using U/V spectrophotometer. Real time PCR was done after RNA extraction at the same day. Mean delta CT for housekeeping genes were 24 indicating average RNA expression. In general, RNAs are stable in serum for 2 years. miRNA and long non-coding RNA which are already most stable forms of RNA were assayed. In general, miRNAs are detected in serum or plasma in a remarkable stable form and can withstand repetitive freezing and thawing cycles. In addition, circulating miRNAs are resistant against RNase-mediated degradation.

Measurement of cardiac Troponin I was conducted in serum samples collected from angina and AMI patients, non-angina and healthy control samples. The manufacturer of cardiac Troponin I is Siemens (adiva contour) . The cardiac Troponin I assay is a 3-site sandwich immunoassay using direct chemillumenscence. The units for the measurements are ng/ml and the 99th percentile upper reference limit of a range 0.04 ng/ml. In some cases, Troponin T was also used.

All statistical data were executed using SPSS 22. A Shapiro-Wilk test was conducted on numerical results to assess if the variables are normally distributed, A Kruskal-Wallis test was performed to compare skewed variables, the gene expression is expressed as median value as data are not normally distributed. Two-tailed P value of 0.05 or less was supposed to be statistically significant.

Additional procedures to detect the circulating Nourin RNAs in cardiac patients’ samples are by measuring exosomes and extracellular vesicles. Furthermore, in addition to the use of the standard qPCR, the Nourin-based RNA network can be detected in cardiac patients’ samples using the gold coated magnetic nanoparticles as a non-PCR based technique. For this Nanogold assay, the Nourin RNAs will be either extracted or measured directly in patients’ samples without purification or pre-amplification. This assay will measure the Nourin RNA panel of markers in various sera samples. In addition, Nourin-based RNA panel of markers can be detected in cardiac patients’ samples using the technology provided commercially, for example by Multiplex miRNA assays measuring the Nourin-based RNA network via total circulating RNAs, Multiplex miRNA assays with FirePlex® particle technology enable simultaneous profiling of 65 miRNAs directly from small amounts of biofluid or FFPE, without RNA purification or pre-amplification. Assays can be customizable for the Nourin-based RNA panel of markers and suitable for both discovery and verification studies. Readout uses a standard flow cytometer. Additionally, sensor chip procedures can be used to detect the Nourin-based RNA network and the Nourin protein including and not limited to Nourin epitope N-f-MII.

Furthermore, the Point-of-Care (POC) procedures can be used to rapidly within 15 minutes detect in cardiac patients’ samples the circulating Nourin RNAs including lncR-CTB89H12.4, has-miRNA-106b, has-miRNA-137, mRNA ANAPC11 and mRNA FTLH-17, as well as the Nourin epitope N-f-MII. The POC diagnostics has been emerged as a promising real-world application. The POC ecosystem is evolving faster than ever and new technology has to fit into a broader landscape. Some of the main advantages of POC diagnostic device include the use of smaller sample volume, lower test costs and faster turn-around-times i.e., 15 minutes vs, 4 hours to 24 hours for PCR. Beside its rapid and precise response, its portability, low cost and non-requirement of specialized equipment are important advantages. The challenge is that the POC devices use smaller sample volumes to achieve the same detection limit as standardized laboratory equipment. It requires the integration of assay chemistry, fluidics, hardware and software.

A POC device can use a chip-based technology to examine different analytes in various samples including blood, urine and tissue biopsies. Microfluidics and biosensor can use numerous materials such as glass, silicon, polymer, and paper for the fabrication of microfluidics-based POC devices along with their wide range of biosensor applications. Recent development in nanomaterials, device design, and microfabrication technologies have made it possible to obtain POC devices with enhanced sensing characteristics. Breakthroughs such as the recently published method of 3D printing microfluidics lab-on-a-chip devices could help lead to cheaper mass-production of diagnostic devices. The use of smartphones paired to microfluidics could enable an increased range and ability of POC testing, with the development of devices such as the TRI analyzer on the horizon, it is possible to achieve limits of detection that are comparable to those obtained for the same assay measured with a conventional laboratory microplate reader, demonstrating the flexibility of the system to serve as a platform for rapid, simple translation of existing commercially available bio sensing assays to a POC setting. POC portable devices identification method can be based on microarray platform require extensive testing and validation comparing the outcome with more traditional methods of detection. Thus, the high-performance RNA-detection methods for all types of clinically relevant RNAs (mRNAs, miRNAs and IncRNAs) are based on molecular-biology techniques including and not limited to qPCR, microarrays, nanoparticles, microfluidics and biosensor.

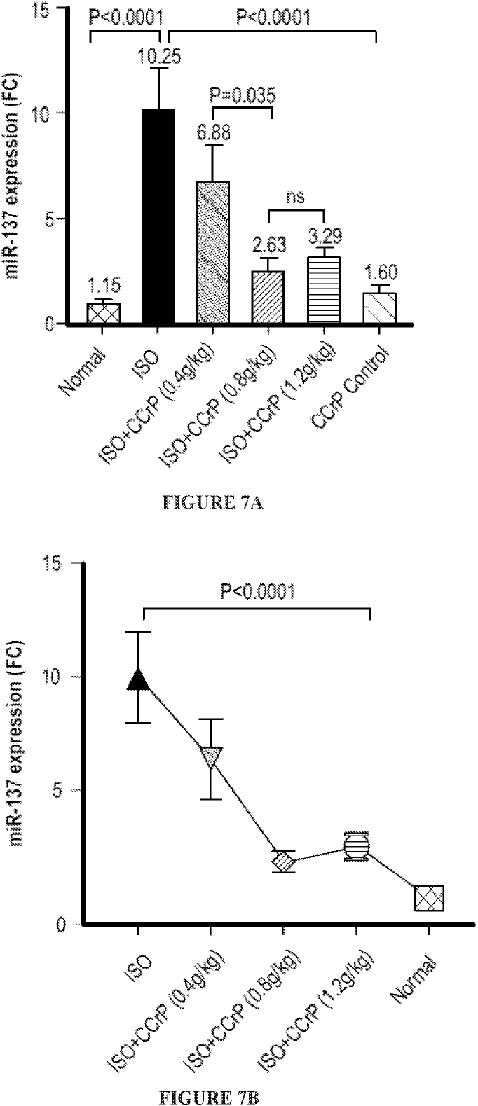

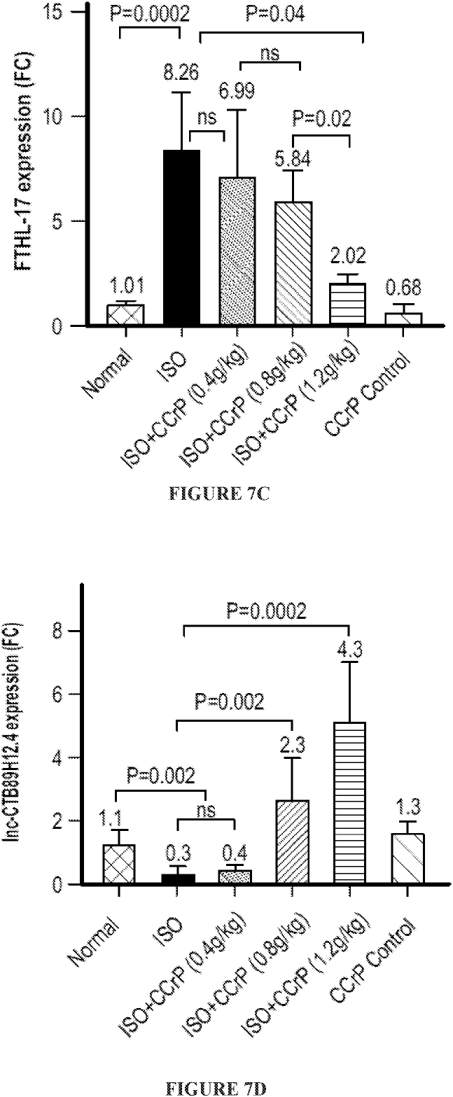

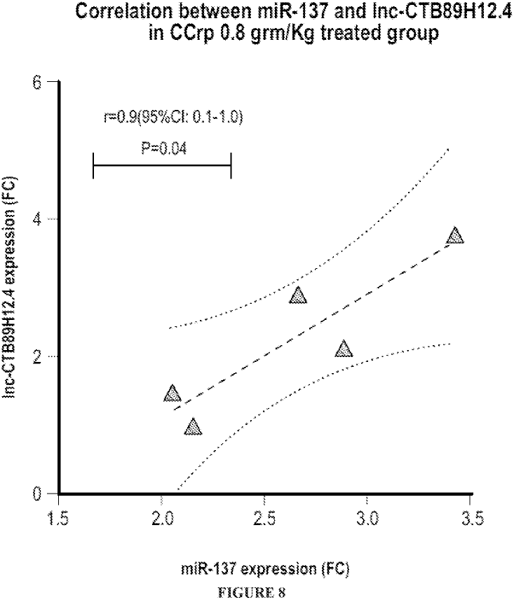

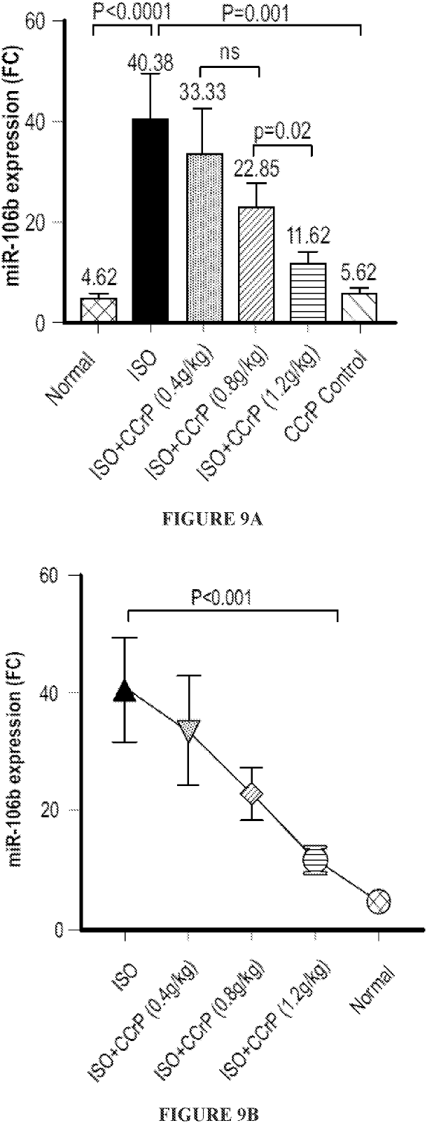

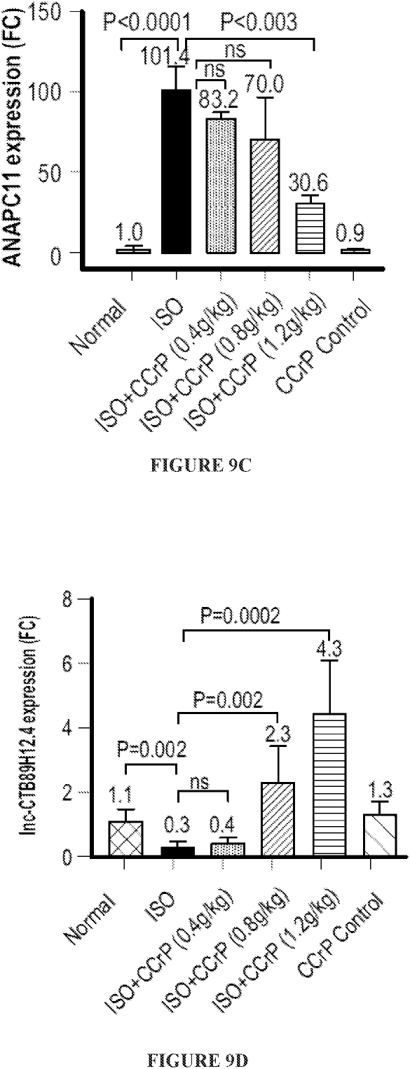

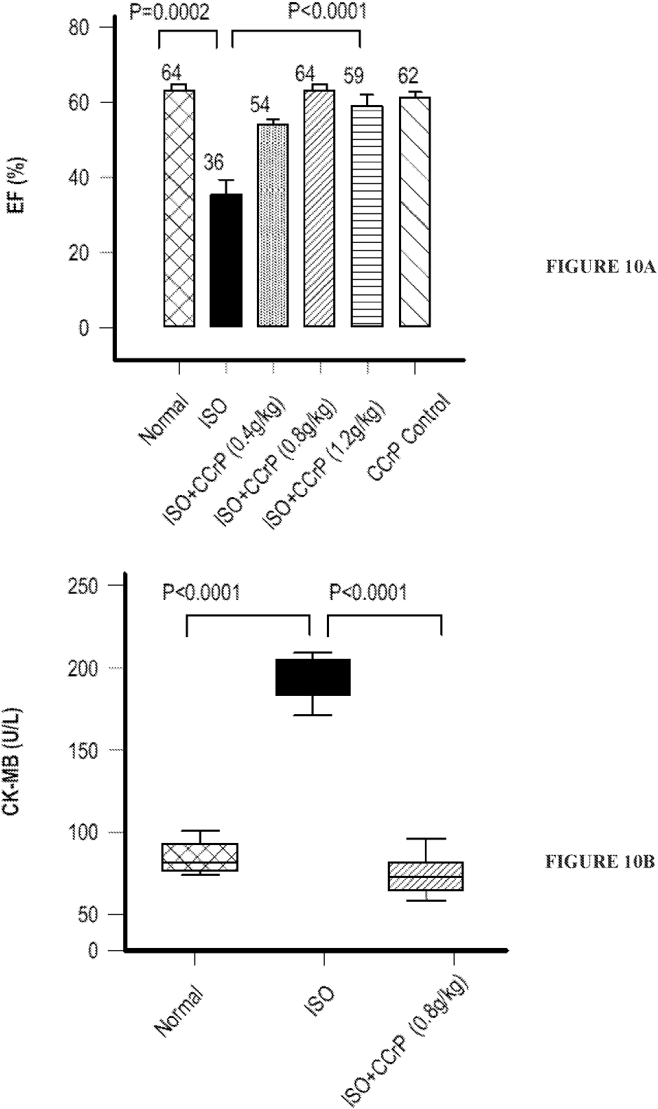

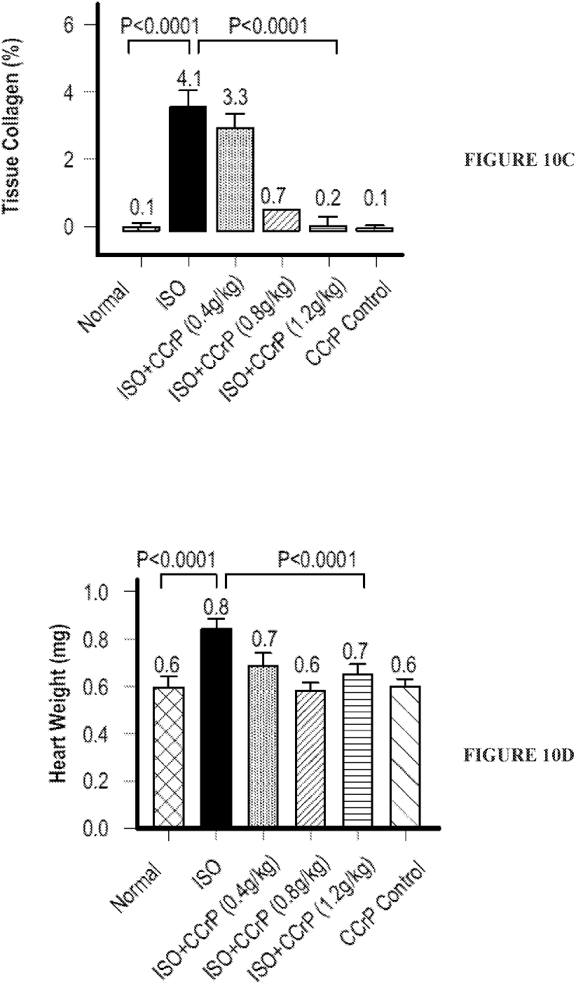

25 male Wistar rats (180-220 g) were used: ISO/saline (n=6), ISO/CCrP 0.4 gm/kg/day (n=3), ISO/CCrP 0.8 gm/kg/day (n=5). ISO/CCrP 1.2 gm/kg/day (n=2), control/saline (n=5), and control/CCrP 0.8 gm/kg/day (n=4). Rats were injected S.C. with ISO for two consecutive days at doses of 85 and 170 mg/kg/day, respectively, then left for an additional 2 weeks. CCrP and saline were injected IP (1 ml) 24 hours and 1 hour before ISO administration, then daily for 2 weeks. Serum creatine kinase-MB (CK-MB) (U/L) measured 24 hours after last ISO injection. After 14 days, gene expression of Nourin-dependent miR-137 and miR-106b and their signaling pathways mRNA-FTLH-17, mRNA-ANAPC11 and IncR-CTB89H12.4 were evaluated by qRT-PCR. After 24 hours of the second and last ISO injection, it was confirmed that the development of myocardial injury by measuring the levels of necrotic biomarker, CK-MB (FIG. 10B). Although the saline-treated ISO rats (ISO/saline) had very high levels of CK-MB indicative of the presence of myocardial injury, CCrP-treated ISO (ISO/CCrP) rats showed low levels of CK-MB, which were comparable to the baseline healthy-saline rats (FIG. 10B). The absence of elevation of CK-MB in ISO/ CCrP rats suggests that CCrP prevented ischemic injury and protected rats from the development of myocardial injury, thus, maintained healthy hearts. After 14 days after the second and last ISO injection (end of study), Nourin-dependent miR-137 and miR-106b and their signaling pathways mRNA-FTLH-17, mRNA-ANAPC11 and IncR-CTB89H12.4 were measured in rat serum samples (n=25). As indicated in FIG. 7A - FIG. 7D, FIG. 8, and FIG. 9A - FIG. 9D, there was a significantly upregulation of gene expression ofNourin-related miR-137, miR-106b, mRNA-FTLH-17 and mRNA ANAPC11 in serum samples from ISO/saline rats, while normal “saline” rats showed very low gene expression. The ISO/saline rats had downregulation of IncR-CTB89H12.4 compared to healthy rats received “saline”.

Specifically, as indicated in FIG. 7A - FIG. 7B, there is a significantly high gene expression level of miR-137 in serum samples collected at day 14 from ISO rats compared to normal rats. The ISO/saline rats had upregulation by 8.91-folds (Mean=10.25) compared to healthy rats received saline (1.15) (p<0.0001). CCrP treatment signicantly (p<0.0001) reduced miR-137 gene expression at doses of 0.4 g/kg, 0.8 g/kg and 1.2 g/kg. CCrP at doses of 0.4 g/kg, 0.8 g/kg, and 1.2 g/kg by 33%, 75% and 68%, respectively. Additionally, CCrP adminstration to healthy rats at 0.8 g/kg did not increase miR-137 gene expression (Mean=1.60) and it was comparable to the level expressed in saline-treated healthy rats (1.15). The ISO/saline rats had upregulation of mRNA-FTHL-17 by 8.17-fold (Mean=8.26) compared to healthy rats received saline (1.01) (p=0.0002) (FIG. 9C). CCrP treatment signicantly (p=0.04) reduced mRNA-FTHL-17 gene expression at doses of 0.4 g/kg. 0.8 g/kg and 1.2 g/kg by 16%, 30% and 75%, respectively. Additionally, CCrP administration to healthy rats at 0.8 g/kg did not increase mRNA-FTHL-17 gene expression (Mean =0.67) and it was comparable to the level expressed in saline-treated healthy rats (1.01). The ISO/saline rats had downregulation of IncRNA-CTB89H12.4 (Mean=0.3) compared to healthy rats received saline (1.1) (p=0.002) (FIG. 9D). CCrP treatment signicantly (p=0.002) increased IncRNA-CTB89H12.4 gene expression at doses of 0.4 g/kg, 0.8 g/kg and 1.2 g/kg by 1.33-fold, 7.66-folds and 14.33-folds, respectively. Additionally, CCrP administration to healthy rats at 0.8 g/kg did not affect IncRNA-CTB89H12.4 gene expression (Mean=1.3), and its expression level is comparable to saline-treated healthy rats (1.1). FIG. 8 shows a correlation analysis which was conducted between miR-137/FTHL-17/lncR-CTB89H12.4 in the ISO (heart failure) rats treated with CCrP (0.8 g/kg). The only significant correlation was found between miR-137 and IncRNA-CTB89H12.4 (p=0.04) in the ISO/CCrP group where reduction of Nourin-related miR-137 is due to the “corrective” effect of CCrP on myocardial ischemia. No significant correlation was detected between miR-137/FTHL-17/ lncR-CTB89H12.4 in the ISO/saline group (p>0.05). Similarly, FIG. 9A - FIG. 9D present gene expression level of Nourin RNA network composed of miR-106b/ANAPC11/ lncR-CTB89H12.4 in the standard isoproterenol (ISO) rat model of HF (ISO/saline) and how the administration of Cyclocreatine Phosphate (CCrP) (ISO/CCrP) inhibited gene expression of Nourin RNA network. (FIG. 9A) and (FIG. 9B) indicate the significantly high gene expression level of miR-106b in serum samples collected at day 14 from ISO/saline rats compared to normal rats. The ISO/saline rats had upregulation by 8.74-folds (Mean=40.38) compared to healthy rats received saline (4.62) (p<0.0001). CCrP treatment signicantly (p<0.001) reduced miR-106b gene expression at doses of 0.4 g/kg, 0.8 g/kg, and 1.2 g/kg by 18%, 44% and 72%, respectively. Additionally, CCrP adminstration to healthy rats at 0.8 g/kg did not increase miR-106b gene expression (Mean=5.62) and it was comparable to the level expressed in saline-treated healthy rats (4.62). The ISO/saline rats had upregulation of mRNA ANAPC11 by 101.4-fold (Mean=101.4) compared to healthy rats received saline (1.0) (p=0.0002) (FIG. 9C). CCrP treatment signicantly (p=0.04) reduced mRNA ANAPC11 gene expression at doses of 0.4 g/kg, 0.8 g/kg and 1.2 g/kg by 18%, 31% and 70%, respectively. Additionally, CCrP adminstration to healthy rats at 0.8 g/kg did not increase mRNA FTLH-17 gene expression (Mean=0.9) and it was comparable to the level expressed in saline-treated healthy rats (1.0). The ISO/saline rats had downregulation of lncRNA-CTB89H12.4 (Mean=0.3) compared to healthy rats received saline (1.1) (p=0.002) (FIG. 9D). CCrP treatment signicantly (p=0.002) increased IncRNA-CTB89H12.4 gene expression at doses of 0.4 g/kg, 0.8 g/kg and 1.2 g/kg by 1.33-folds, 7.66-folds and 14.33-folds, respectively. Additionally. CCrP adminstration to healthy rats at 0.8 g/kg had IncR-CTB89H12.4 gene expression (Mean=1.3) had a comparable level of expression as the saline-treated healthy rats (1.1). No significant correlation was detected between miR-106b/ANAPC11/ lncR-CTB89H12.4 in the ISO/saline group (p>0.05). Similarly, no significant correlation was detected between miR-106b/ANAPC11/ lncR-CTB89H12.4 the ISO/CCrP at dose of 0.8 g/kg (p>0.05).

Additionally, it was evaluated if the toxic effect of CCrP administration daily for 14 days to normal rats and whether CCrP would cause myocardial injury and stimulates expression of Nourin-dependent RNA network. As indicated in FIG. 7A - FIG. 7D and FIG. 9A - FIG. 9D, there was very low gene expression in CCrP control rats and there was no difference between CCrP control and normal saline control rats, suggesting lack of CCrP cardiac toxicity. Recent evidence suggests that miRNAs are involved in the development and progression of HF. Several miRNAs have been identified as potential candidates that could be used as diagnostic biomarkers for HF to provide valuable clinical information. Additionally, they may be important tools in monitoring the progress of therapeutic treatments, since medical interventions are also associated with changes in miRNA levels. Experimentally, circulating levels of miR-423-5p and miR-106 were markedly increased in hypertension-induced HF, which was confirmed via RT-qPCR analysis of plasma RNA from hypertensive rats. Additionally, miR-106b is upregulated in cardiac tissue of patients with dilated cardiomyopathy and that miR-106b and miR-15b modulate apoptosis and angiogenesis in myocardial infarction. The expression of miR-137 was also detected by RT-qPCR and western blot analysis in spontaneously hypertensive rat hearts. miR-137 may promote cardiac remodeling in these rats by upregulation of Ang II and the TGF-B1/Smad3 signaling pathway; in addition, captopril intervention can inhibit miR-137 expression. Therefore, miR-137 not only indicates the presence of high blood pressure, it may also reflect its severity. These results indicate that several miRs can reflect disease progression to a certain extent, and may be used as biomarkers of hypertensive HF. Levels of serum miR-1 were also positively associated with myocardial infarct size. In post-AMI patients, miR-1 was significantly correlated with (a) the absolute change in infarct volume, (b) showed a trend for positive correlation with LV ejection fraction, and (c) was associated with AMI mortality. AMI patients, also, had significantly higher levels of plasma miR-21, compared to healthy controls. miR-21 was shown to be a novel biomarker that was predictive of LV remodeling after AMI, which correlated with several traditional markers of AMI: creatine kinase-MB (CK-MB), creatine kinase (CK) and cardiac troponin I (cTnI), with comparable diagnostic accuracy.

In summary, the disclosure according to the present invention indicates:
1) the higher expression level of Nourin-related miR-137 observed in HF rats compared to normal “saline” group with a highly significant difference (p<0.0001), could be explained by its over expression in response to ischemia;
2) similarly. Nourin- related miR-106b expression was markedly increased in the HF model compared to normal “saline” group with a highly significant difference (p<0.0001), which explains the effect of myocardial ischemia on the release of the inflammatory mediator Nourin protein as a consequence effect of overexpression of miR-106b;
3) after an ischemic event in the ISO / HF rat model, there was downregulation of IncR-CTB89H12.4 and up-regulation of miR-137 and miR-106b which activated mRNA FTHL-17 and mRNA ANAPC11, respectively, resulting in an increase in translation of the Nourin protein;
4) Nourin-dependent RNA network expression level in ISO rats was compared to ISO/CCrP treated rats using 3 doses of CCrP (0.4 gm/kg/day, 0.8 gm/kg/day and 1.2 gm/kg/day);
5) significant reduction of the expression was detected in CCrP-treated HF model in a dose response manner with maximum efficiency with the effective dose of 0.8 g/kg/day;
6) reduction of Nourin- related gene expression observed after CCrP treatment, is due to the “corrective” effect ofCCrP on myocardial ischemia;
7) the effect of CCrP on Nourin RNA network gene expression was comparable at the 3 doses:
   a) downregulation of miR-137 gene expression, by CCrP at doses of 0.4 g/kg, 0.8 g/kg, and 1.2 g/kg was 33%, 75% and 68%, respectively,
   b) downregulation of mRNA-FTHL-17 gene expression by CCrP at doses of 0.4 g/kg, 0.8 g/kg, and 1.2 g/kg was 16%, 30% and 75%, respectively,
   c) downregulation of miR-106b gene expression by CCrP at doses of 0.4 g/kg, 0.8 g/kg and 1.2 g/kg was 18%, 44% and 72%, respectively,
   d) downregulation of mRNA-ANAPC11 gene expression by CCrP at doses of 0.4 g/kg, 0.8 g/kg and 1.2 g/kg was 18%, 31% and 70%, respectively, and
   e) upregulation of IncRNA-CTB89H12.4 gene expression by CCrP at doses of 0.4 g/kg, 0.8 g/kg and 1.2 g/kg was 1.33-fold. 7.66-fold and 14.33-fold, respectively;
8) no observed toxic effect of CCrP when administered daily for 14 consecutive days to normal healthy rats. CCrP did not cause myocardial injury and did not stimulate expression of Nourin-dependent RNA network. There was a very low gene expression in CCrP-treated healthy rats which was not different than normal saline rats, indicating lack of CCrP cardiac toxicity;
9) the upregulation of Nourin RNA network can be used as early diagnostic and prognostic biomarkers for cardiomyocyte injury and inflammation in HF patients;
10) CCrP prevented ischemic injury and inhibited gene expression of the Nourin molecular RNA network in ISO / HF rats; and
11) Nourin RNA network can be used as molecular therapeutic targets to prevent cell injury and inflammation in patients with ischemic diseases using the bioenergetic drug, CCrP.

### Example 4 - The Bioenergetic Drug, Cyclocreatine Phosphate Is A “Novel Mechanism” to Prevent the Development of Heart Failure in ISO Rat Model by Preventing Ischemic Injury, Reducing Fibrosis and Remodeling, Resulting in Rejuvenation of Cardiac Function and Restorion of Normal Physical Activities.

Myocardial infarction is the most common cause of heart failure. The recent improvements in medical and surgical treatments of acute coronary syndrome are leading to an increasing number of “survivors” who are then developing heart failure, which characterized with reduced left ventricular myocardial function, dyspnea (difficulty breathing) and limited exercise tolerance. Depending on the time of onset, HF with reduced ejection fraction (EF) of 40%, is classified as acute or chronic and patients.

Virtually all episodes of ACS, including UA and both STEMI and NSTEMI, are associated with the loss of myocardiocytes, edema, inflammation, fibrosis, and cardiac remodeling, which all together represent the leading pathophysiological mechanisms of HF. A hallmark feature of ventricular remodeling is deposition of excessive extracellular matrix. This surplus extracellular matrix, which constitutes scar or fibrosis, promotes both contractile dysfunction and rhythm disturbances. As a result, cardiac fibrosis contributes to morbidity and mortality in many forms of heart disease. Indeed, the amount of fibrotic scar in the myocardium correlates strongly with the increased incidence of arrhythmias and sudden cardiac death. Extracellular matrix deposition and fibrosis formation occur through the action of cardiac fibroblasts. In the setting of pathological stress, fibroblasts proliferate and differentiate into myofibroblasts, thereby gaining the capacity to contract and secrete collagen I, collagen III, and fibronectine. Both collagenous and myofibroblasts propagate the arrhythmic phenotype of the remodeled heart.

Isoproterenol (ISO) is a beta-adrenergic agonist which in high doses cause pathologic and molecular changes in rat heart that are similar to myocardial injury in humans. It causes coronary vasoconstriction, exaggerates myocardial Ca2+ influx and causes shunting of blood away from the subendocardial layer, producing subendocardial ischemia cellular ATP depletion . ISO undergoes autoxidation, generating highly toxic ROS, which activate apoptotic pathways in the myocardium which in turn lead to contractile dysfunction and cardiomyocyte cell death. Therefore, high doses of ISO are used to induce experimental myocardial injury and to validate the effectiveness of test drug against ischemia-induced HF.

Demand ischemia causes irreversible myocardial injury through exhaustion of cellular ATP. It was demonstrated that enhancing myocardial ATP stores during ischemia using Cyclocreatine and its water-soluble salt Cyclocreatine Phosphate, prevents myocardial injury and maintains cardiac contractility in a variety of models. It was therefore tested if the hypothesis that CCrP administration will prevent ischemic injury and the subsequent development of heart failure in standard isoproterenol (ISO) rat model. 25 male Wistar rats (180-220 g) were used: ISO/saline (n=6), ISO/CCrP at three doses. 0.4 gm/kg/day (n=3), 0.8 gm/kg/day (n=5) and 1.2 gm/kg/day (n=2), control/saline (n=5), and control/CCrP 0.8 gm/kg/day (n=4). From our previous studies. CCrP at 0.8 gm/kg/day is the most effective dose to prevent ischemic injury and restores cardiac function. Rats were injected S.C. with ISO for two consecutive days at doses of 85 and 170 mg/kg/day, respectively, then left for 2 weeks . CCrP and saline were injected IP (1 ml) 24 hours and 1 hour before ISO administration, then daily for 2 weeks. Serum CK-MB (U/L) measured 24 hours after last ISO injection. After 14 days, ECHO analysis for Ejection Fraction (EF%) was conducted, as well as heart weight (mg), histologic analysis for fibrosis and deposition of collagen. Mean ± S.E.M and one-way ANOVA analysis were used.

FIG. 10B shows evidence of myocardial injury after 24 hours by high elevation of CK-MB in ISO rats (206.20±6.25), while significant protection was seen in ISO/CCrP rats (70.67± 5.79) at 0.8 g/kg (p<0.0001). ISO/CCrP rats had CK-MB level comparable to control saline (82.60±5.2), indicating that CCrP treatment prevented myocardial ischemic injury.

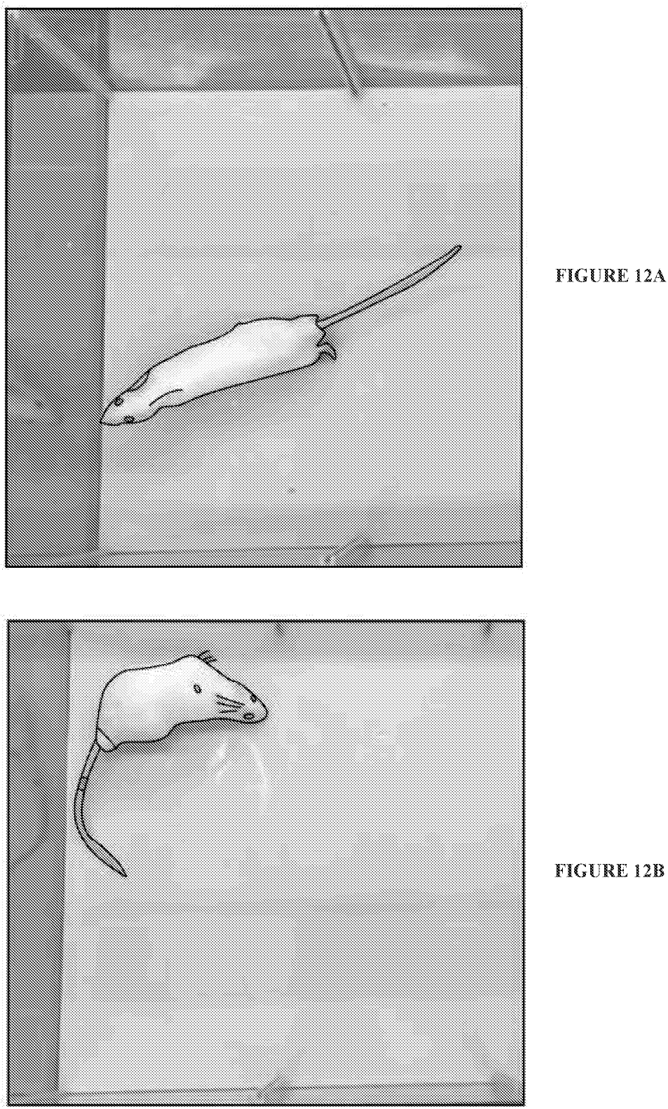

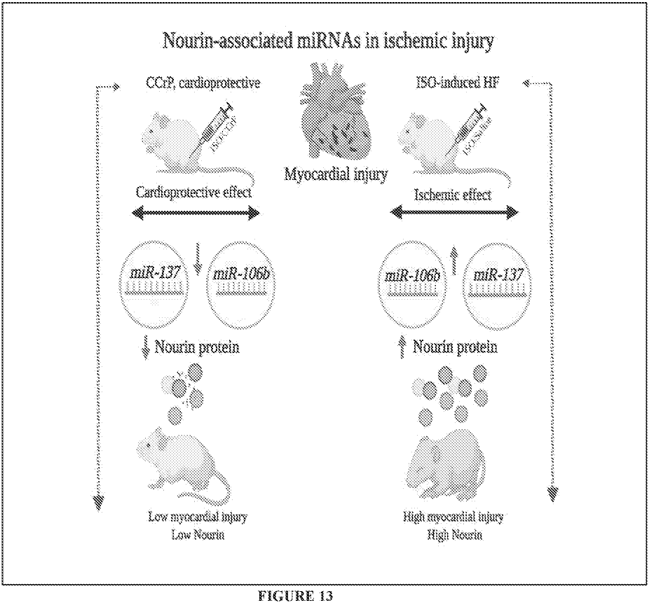

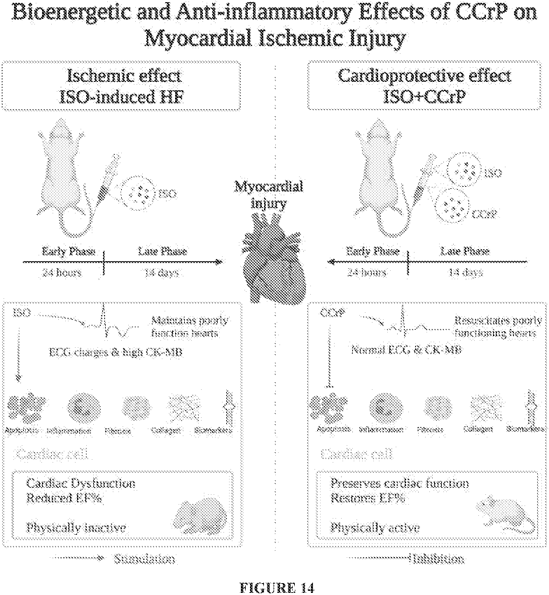

FIG. 10A summarizes results of EF% measured in various groups after 14 days using rat ECHO. While ISO rats showed significant drop in EF% of 36 indicative of acute heart failure, ISO/CCrP rats showed normal EF% of 64 at 0.8 g/kg/day (p<0.0001), which was comparable to control saline EF% of also. 64. Furthermore, ISO/CCrP group (n=5) (FIG. 12B) showed “high physical activity” at day 14 before sacrifice: activity which is comparable to control healthy “saline” rats. ISO/saline rats (n=6), on the other hand showed “low physical activity” and rats mainly stayed in place (FIG. 12A). These results indicate that treating ISO rats with CCrP prevented the development of heart failure and restored normal heart function and physical activities. Similarly, treating ISO/saline rats with CCrP at doses of 0.4 g/kg and 1.2 g/kg continued to show good EF% of 54 and 59. respectively, further confirming the cardioprotective benefits of CCrP in preventing the development of acute heart failure . Finally, normal rats treated daily for 14 days with CCrP at .8 g/kg also had normal EF% of 61 which was comparable to control saline EF% of 64, suggesting lack of toxicity of CCrP on heart function.

FIG. 10C indicates that while ISO/saline rats showed significant high collagen % of 4.1, ISO/CCrP rats showed low collagen % of 0.7 at 0.8 g/kg/day (p<0.0001), which was comparable to control saline collagen % of 0.1. Furthermore, treating ISO rats with CCrP at 1.2 g/kg continued to show low collagen % of 0.2 compared to ISO/saline rats (4.1) (p<0.0001), further confirming the cardioprotective benefits of CCrP in preventing cardiac remodeling. Only partial reduction of collagen % of 3.3 was seen using CCrP at doses of 0.4 g/kg. Additionally, normal rats treated daily for 14 days with CCrP at 0.8 g/kg also had normal collagen % of 0.1, which was comparable to control saline collagen % of 0.1, suggesting lack of toxicity of CCrP on heart remodeling (FIG. 10C). Finally, normal rats treated daily for 14 days with CCrP at 0.8 g/kg also had normal collagen % of 0.1, which was comparable to control saline collagen % of 0.1, suggesting lack of toxicity of CCrP on heart remodeling (FIG. 10C).

FIG. 10D shows a significant increase in heart weight (0.8 mg) in ISO rats compared to normal saline rats (0.6 mg) (p<0.0001), suggesting heart damage and edema. On the other hand, ISO/CCrP rats showed significantly lower heart weight at doses of 0.4 g/kg (0.7 mg), 0.8 g/kg (0.6 mg), and 1.2 g/kg (0.7 mg) (p<0.0001), confirming the cardioprotective benefits of CCrP in protecting against heart damage and weight gain. Interestingly, the ability of CCrP to prevent heart weight gain in rat model of heart failure supports our previous reporting in FIG. 42B demonstrating that CCrP reduced the gain of heart weight after 6 hours of cold storage. The observed reduction of heart edema in the CCrP hearts is crucial for the restoration of contractile function during reperfusion. Finally, normal rats treated daily for 14 days with CCrP at 0.8 g/kg showed normal heart weight of 0.6 mg, which was comparable to normal saline rats (0.6 mg), suggesting lack of toxicity of CCrP on the heart by maintain normal heart weight.

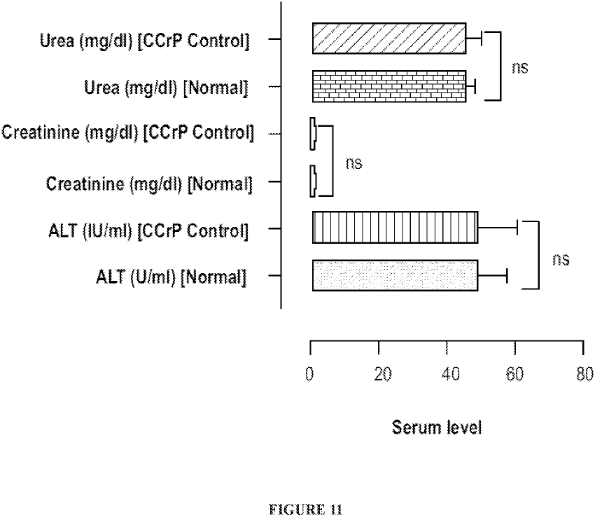

FIG. 11 indicates the safety of CCrP at a dose of 0.8 g/kg, where healthy rats were treated daily with CCrP for 14 days and showed no toxicity in heart, liver and renal function. There was no significance difference between serum levels of normal rats treated with saline or CCrP for the levels of Nourin gene-based RNA network, liver enzyme ALT, kidney Creatinine and Urea, as well as EF%, collagen% and heart weight.

In summary, this study indicates that:
a. ISO/saline rats showed:
   i. high elevation of CK-MB
   ii. significant drop in EF%
   iii. marked increase in collagen deposition
   iv. marked fibrosis
   v. significant increase in heart weight
   vi. increase in expression level of Nourin gene-based RNA network (miR-137, miRNA-106b, mRNA-FTLH-17, mRNA-ANAPC11, and IncR-CTB89H12.4)
   vii. toxicity of heart
   viii. very low physical activity at day 14.
b. ISO/CCrP rats showed:
   i. no elevation of CK-MB
   ii. no drop in EF%
   iii. no collagen deposition
   iv. no fibrosis
   v. no increase in heart weight
   vi. no increase in expression level of Nourin gene-based RNA network (miR-137, miRNA-106b, mRNA-FTLH-17, mRNA-ANAPC11, and IncR-CTB89H12.4)
   vii. no toxicity in heart
   viii. normal physical activity at day 14.
c. CCrP administration in the ISO rat model of HF likely prevented the development of HF by:
   i. preventing ischemic injury as indicated by normal level of the cardiac biomarker CK-MB;
   ii. preventing cardiac remodeling by reducing fibrosis and collagen deposition;
   iii. preventing cardiac injury and gain in heart weight; and
   iv. restoring normal ejection fraction, cardiac function
   v. restoring organ rejuvenation and physical activities.
d. CCrP not only prevented ischemic injury and the “development” of myocardial injury (MI) by 24 hours after ISO administration, but, also, protected cardiac tissue from remodeling and prevented the “progression” of MI to acute heart failure at day 14.
e. Thus, the bioenergetic CCrP is a promising first-in-class novel mechanism of cardioprotection that prevents ischemic injury, as well as prevents development and progression of heart failure, thus, rejuvenate cardiac function and restores normal physical activity.

The gene expression of Nourin miRNAs miR-137 and miR-106b-5p is thus upregulated at day 14 in ISO/saline “HF rats” and that the administration of CCrP reduces myocardial injury and the expression levels of Nourin miRNAs in ISO/CCrP “non-HF rats.” FIG. 14 indicates that ISO-induced myocardial ischemic injury resulted in upregulation of miR-137 and miR-106b-5p with a likely increase of translation and production of the Nourin protein, as has been previously demonstrated using the intact canine models of AMI and bypass surgery, where the circulating Nourin protein was markedly elevated.

### Example 5 - CCrP Administration Preserved ATP Levels and Restored Heart Function in HF (ISO) Model.

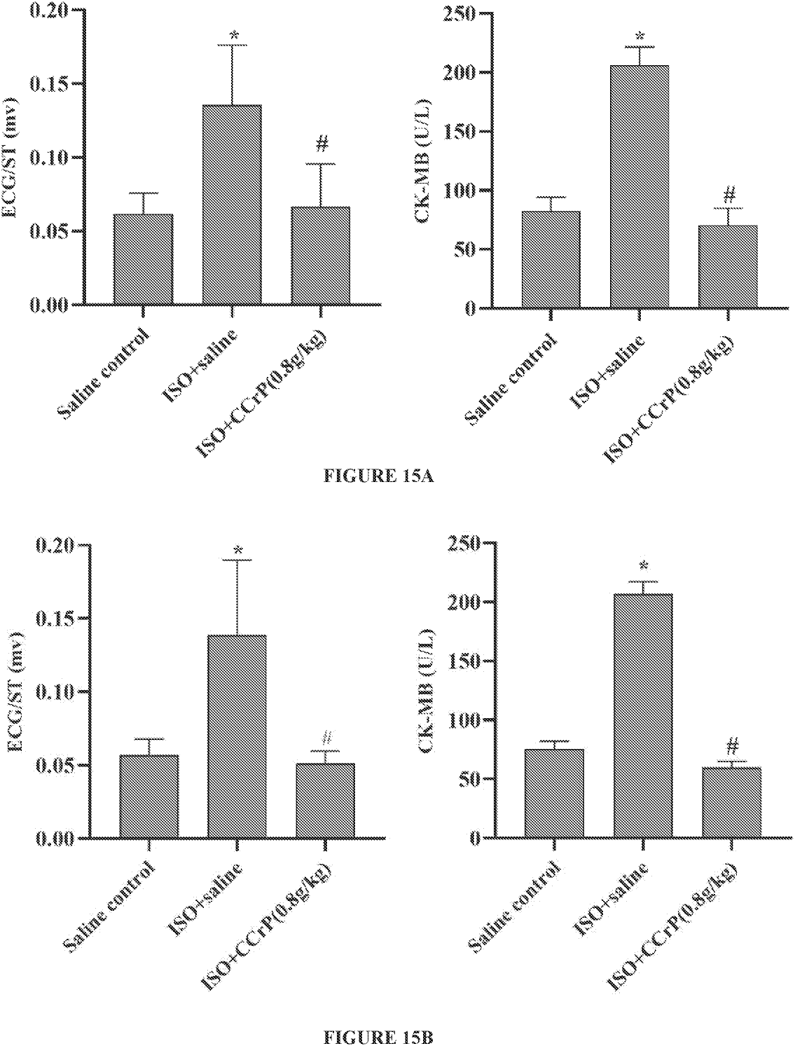

In this example, the cardioprotective benefits of CCrP in the standard ISO rat model of demand ischemia-induced HF was explored. The hypothesis that CCrP treatment will prevent ischemic injury and development of HF, when administered prophylactically, and will salvage poorly functioning hearts when administered therapeutically was tested. As depicted in FIG. 15, CCrP administered prophylactically: 1) prevented ischemic injury in the acute phase of 24 hours after the second ISO injection as shown by normal ECG/ST and CK-MB levels; and 2) significantly protected against the development of HF after 14 days by reducing the downstream harmful events, through marked reduction of cardiac inflammation, apoptosis, biomarkers (hs-TnI, BNP, TNF-α, and TGF-β), remodeling (fibrosis/collagen deposition), and heart weight, but increased expression of eNOS and the cardiac conduction and function, connexin-43 β-actin with the restoration of normal ejection fraction, cardiac function, and physical activity. When administered therapeutically, CCrP resuscitates poorly functioning hearts in the acute phase of 24 hours after the second ISO injection, and the observed normal cardiac function was sustained for an additional 14 days, with the restoration of normal physical activity. This example shows that CCrP administration prevents ischemic injury and the development of heart failure, as well as salvages poorly functioning hearts in the standard ISO rat model of demand ischemia-induced stress cardiomyopathy. CCrP cardioprotection was observed in both the early acute phase during the first 24 hours and the late phase after 14 days.

The model provides evidence that myocardial injury was documented in the ISO/saline rats 24 hours after the second ISO injection by the detection of a high elevation of CK-MB levels (2.78-fold, p<0.05) and ECG/ST changes (2.70-fold, p<0.05) compared to the saline/control group. Results demonstrate that CCrP treatment both prophylactically as seen with FIG. 15A and therapeutically as seen with FIG. 15B prevented myocardial ischemic injury, as indicated by normalized ST segment together with CK-MB level compared to those detected in the ISO/saline rats (p<0.05).

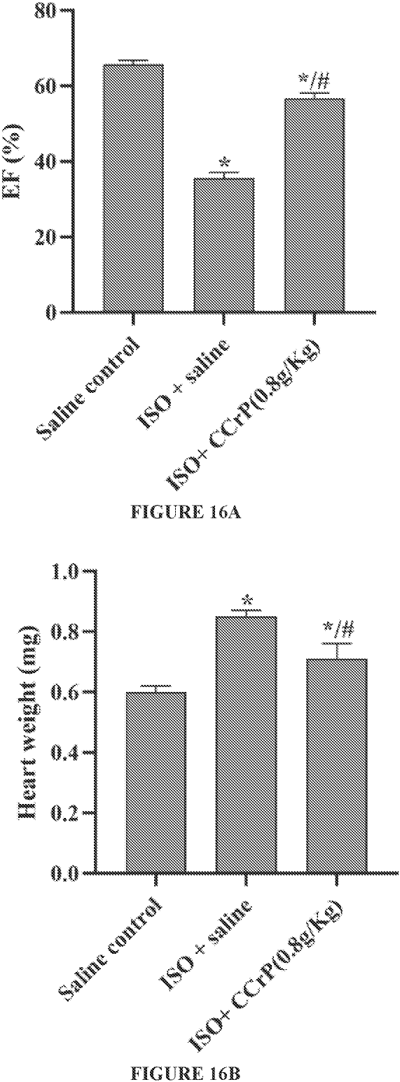

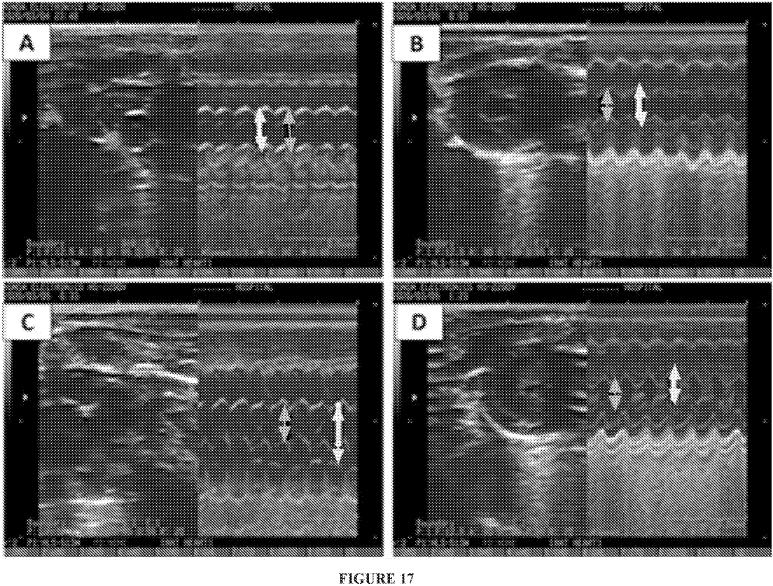

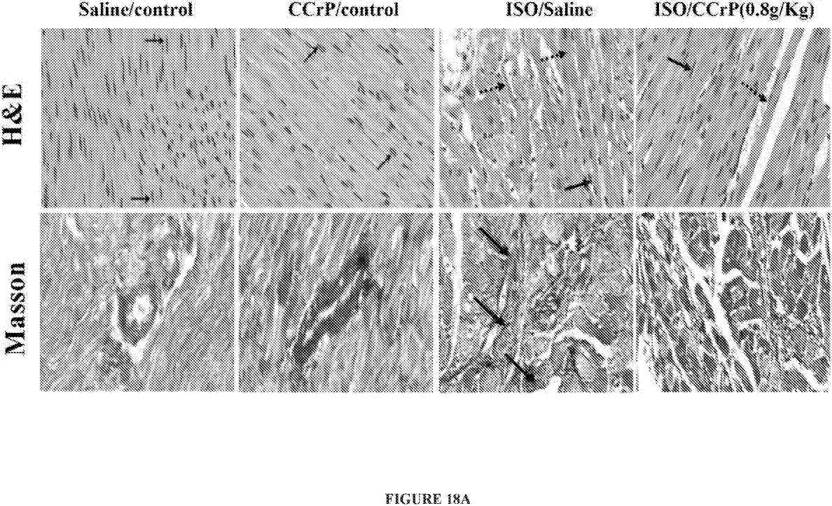

CCrP further prevented cardiac dysfunction and led to an increase in Heart Weight Index (HWI) as seen with FIG. 16A and FIG. 16B, respectively. When assessing cardiac function in terms of ejection fraction percent (EF%) as shown in FIG. 16A, after 14 days, ISO/saline rats showed a significant drop in EF% (35.57%±2.25) compared to saline/control rats (63.87%±0.29) (p<0.05). Whereas, rats in the ISO/CCrP group showed normal EF% of 63.67%±0.13 at the dose of 0.8 g/kg/day, compared to the ISO+saline group (35.57%±2.25) (p<0.05). Similar restoration of normal EF% was seen in ISO/CCrP rats that received CCrP at doses of 0.4 g/kg/day (53.93±0.77) and 1.2 g/kg/day (59.43±1.43) (p<0.05). Further, the administration of CCrP to healthy rats at a dose of 0.8 g/kg/day for 14 days showed a normal EF% of 61.57%±0.64 (p<0.05), suggesting a lack of heart toxicity. Next, the heart weight index (HWI) as demonstrated in FIG. 16B showed that ISO/saline rats showed a significant increase in HWI (0.85±0.02 mg) compared to saline/ control (0.60±0.02 mg) (1.32-fold, p<0.05), indicating myocardial hypertrophy. On the other hand, ISO/CCrP rats at 0.8 g/kg/day reduced HWI by 83.25% of that of ISO/saline rats (p<0.05). Additionally, CCrP treatment at 0.4 and 1.2 g/kg/day showed reduction of HWI by 78.88% and 80.09%, respectively.

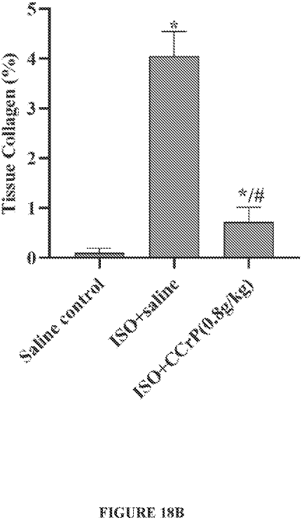

Additionally, an ECHO analysis was performed for verification, and images are presented in FIG. 17 as panels A to D. FIG. 17 revealed a significant increase in left ventricular end-diastolic diameter (LVEDD) (1.30-fold) (p<0.05), left ventricular end-systolic diameter (LVESD) (1.66-fold) (p<0.05), and E/A ratio in the ISO-induced HF rat model (FIG. 17, panel C) compared to saline/control rats (FIG. 17, panel A). Treatment with CCrP at a dose of 0.8 g/kg/day in the ISO-induced HF model reverted LVEDD and LVESD to normal conditions (FIG. 17, panel C, and FIG. 17, panel D), with EF% values of 63.67%±0.13 comparable to saline/control rats (FIG. 17, panel A). Further, healthy rats received CCrP at a dose of 0.8 g/kg/day for 14 days showed normal LVEDD (7.93±0.03) and LVESD (5.53±0.09) comparable to those seen in saline/ control rats, suggesting lack of heart toxicity by CCrP administration (FIG. 17, panel B).

It was furhter observed that CCrP reduced cardiac inflammation and remodeling evaluated in terms of Fibrin and Collagen deposition as shown in FIG. 18A and FIG. 18B. Histopathological analysis of hematoxylin-eosin (H&E) and Masson’s trichrome-stained heart sections was conducted by two independent pathologists blinded to the various groups. Results indicated the detection of a marked cardiac inflammatory response and an intense increase in fibrin and collagen deposition in ISO/saline rats (score: +2 to +3), which were not seen in ISO/CCrP rats at the dose of 0.8 g/kg/day (score: 0 to +1) as shown in FIG. 18A. Analysis further showed extensive fibrous deposition in ISO/saline rats, while ISO/CCrP rats showed delicate fibrous tissue between the myocardial bundles, almost close to normal. The quantitative percentage of tissue collagen deposition as presented in FIG. 18B. ISO/saline rats showed a marked increase in collagen deposition (4.05-fold) (p<0.05), while CCrP at a dose of 0.8 g/kg/day showed strong protection by inhibiting collagen deposition by 83% as shown in FIG. 18B. At a dose of 1.2 g/kg/day collagen deposition was inhibited by 94%. Fibrin and collagen deposition were undetectable in the saline/control rats (refer FIG. 18A and FIG. 18B). Similarly, both fibrin and collagen deposition were undetectable in the CCrP control rats where healthy rats received CCrP at a dose of 0.8 g/kg/day for 14 days, suggesting lack of heart toxicity by CCrP administration.

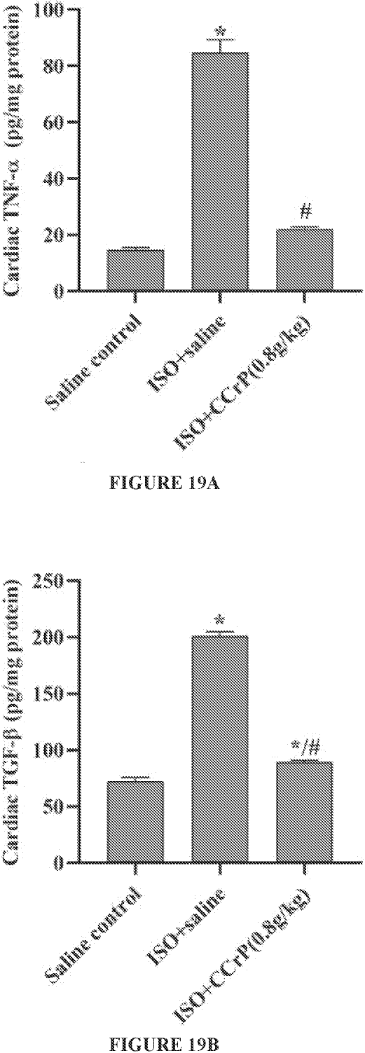

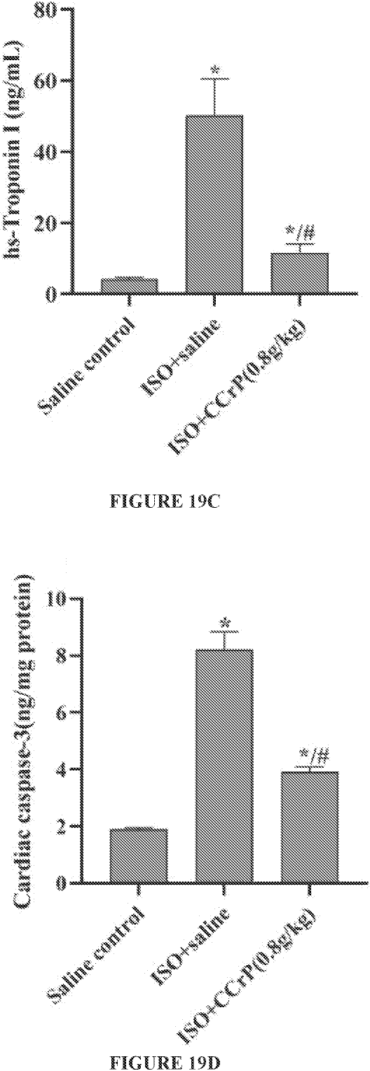

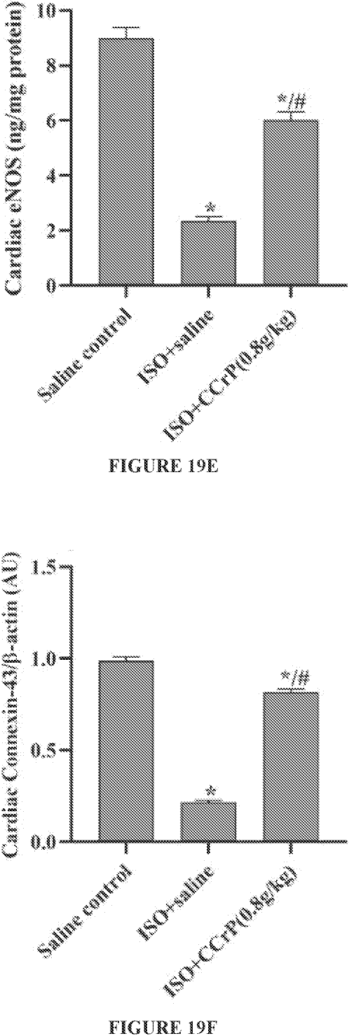

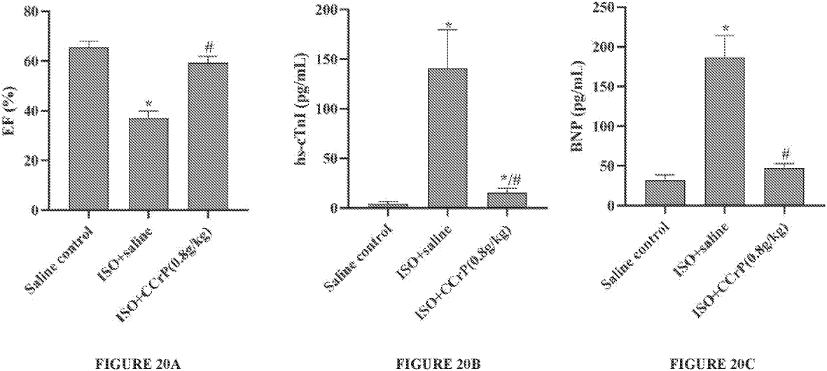

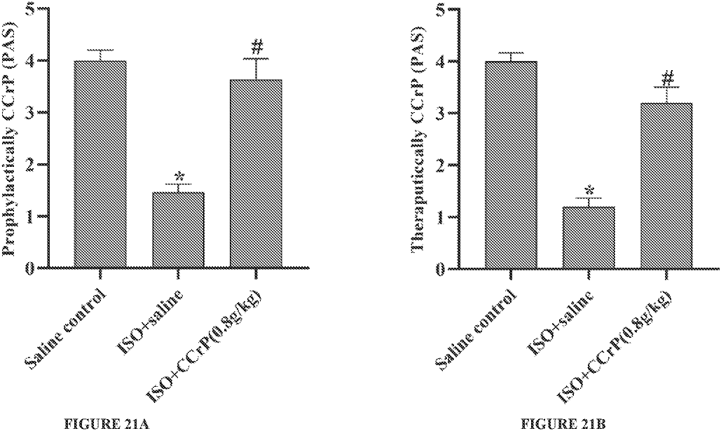

ISO/saline rats further showed marked elevation of TNF-α, TGF-β, hs-cTnI, and caspase-3 contents and reduction in eNOS and the cardiac conduction and function, connexin-43 β-actin as shown in FIG. 19A to FIG. 19F. Isoproterenol-treated rats showed a substantial elevation in cardiac TNF-α (4.2-fold) as shown in FIG. 19A, TGF-β (2.9-fold) as shown in FIG. 19B, hs-cTnI (16.6-fold) as shown in FIG. 19C, and caspase-3 contents (5.4-fold) as shown in FIG. 19D compared to saline/control rats, indicating stimulation of fibrotic and inflammatory pathways, as well as myocardial injury and apoptosis. Such a rise was markedly mitigated (50% to 80%) by CCrP treatment at the effective dose of 0.8 g/kg/day when compared to the levels detected in ISO-saline rats (FIG. 19). Additionally, ISO/saline rats showed a marked decline in cardiac eNOS contents (32.8%) as shown in FIG. 19E and connexin-43 β-actin expression (21.8%) as shown in FIG. 19F. CCrP treatment at a dose of 0.8 g/kg/day markedly enhanced expression of cardiac eNOS (2.74-fold) and connexin-43 β-actin contents (3.74-fold) compared to ISO/saline rats. Further, CCrP treatment at doses of 0.4 g/kg/day and 1.2 g/kg/day significantly decreased levels of TNF-α. TGF-β, and hs-cTnI, and caspase-3 contents by up to 80%, and increased expression of cardiac eNOS (up to 2.38-fold) and connexin-43 β-actin contents (up to 3.41-fold) when compared to ISO/saline rats.

FIG. 20A to FIG. 20C indicate that therapeutically-administered CCrP salvages poor heart function and sustains normal cardiac function. Therapeutically-administered CCrP at a dose of 0.8 g/kg/day showed quick restoration of poorly functioning hearts in rats with high elevation of CK-MB and ECG/ST changes observed in the acute phase after 24 hours after the second ISO injection (FIG. 16). The observed preservation of heart function in the ISO/CCrP rats was sustained over the long term of an additional 14 days, as indicated by normal EF% (56.68±1.42%) compared to the significantly dropped EF% in ISO/saline rats (35.58±1.53%) (p<0.05) (FIG. 7a). Saline/control rats showed normal EF% of 65.70±1.07% as shown in FIG. 20A. CCrP treatment at 0.8 g/kg/day also mitigated the high serum levels of hs-cTnI (pg/mL) in ISO/saline rats (148±11.16) (36.9-fold) compared to saline control (4.71±0.74) (p<0.05), where ISO/CCrP rats showed reduced levels of hs-Tnl (21.08±2.99) (p<0.05) as shown in FIG. 20B. Similarly, ISO/saline showed elevated serum levels of BNP (pg/ml) (185±10.39) (5.7-fold) compared to saline control (32.45±2.32), where CCrP treatment at 0.8 g/kg/day showed reduced levels of hs- BNP (57.71±4.11) (p<0.05) as shown in FIG. 20C. The marked rise in serum levels of hs-cTnl and BNP confirms the presence of cardiac dysfunction and HF as shown in FIG. 20C. Both increases were significantly hampered by CCrP treatment, suggesting the ability of CCrP to salvage poorly functioning hearts from stress cardiomyopathy both in the acute and late phases of the disease.

Further, the example provides that CCrP restores normal physical activity as observed with FIG. 21A and FIG. 21B where after 14 days. ISO/saline rats showed low physical activity scores in both Prophylactically (as shown in FIG. 21A) and therapeutically (as shown in FIG. 21B) administered CCrP. while ISO/CCrP rats showed normal physical activity.

### Example 6 - Clinical Applications of Nourin Protein and Its Regulatory RNA Molecular Network as “Diagnostic” and “Prognostic” Biomarkers for HF and Aging- and IschemiaRelated- Neuro Diseases.

The immune system plays a significant role in post-ischemic cardiac inflammation and ventricular remodeling, and its persistent activation may lead to long-term cardiac injury. MicroRNAs are small non-coding RNAs present in circulation and regulate expression of multiple genes involved in atherogenesis and myocardial ischemia. miRNA-expression profiles are novel diagnostic and prognostic biomarkers for multiple human diseases due to their remarkably high stability in body fluids. They are also easy to obtain through non-invasive methods, are highly sensitive to early detection and have high specificity to different disease entities. Both Nourin-related miRNAs have specific roles in myocardial ischemia, where miR-137 is a marker of cell injury and miR-106b is a marker of inflammation. Thus, using both markers with different modes of action increases diagnostic accuracy. In the present disclosure, using standard rat ISO model of HF: (a) the association between serum Nourin RNA molecular network and, myocardial injury (CK-MB), left ventricle ejection fraction, cardiac fibrosis, remodeling, and heart weight, and (b) whether circulating Nourin level will be reduced by treating ISO rats with the cardioprotective, Cyclocreatine Phosphate (CCrP) were evaluated. Nourin RNA molecular network was highly expressed in response to ischemic cardiac injury, and were not expressed in healthy hearts. The Nourin-related miR-137 is a marker of cell damage, while miR-106b is a marker of inflammation and their signaling pathways include: mRNA-FTHL-17, mRNA-ANAPC11 and lncR-CTB89H12.4. miR-106b is expressed if ischemic myocardial cell damage is associated with an inflammatory response. miR-137 is first expressed in response to injury followed by the expression of miR-1 06b for tissue inflammation. miR-137 and miR-106b are not expressed in normal healthy tissues and only baseline values were detected. Fast release of Nourin-related miR-137 and miR-106b which are specific of ischemic injury and inflammation, will allow them to be used as cardiac markers accuracy than each alone. Their circulating level of expression can indicate the degree of myocardial cell damage and inflammation, thus classify the degree of ischemia as: low, medium and severe.

As seen in the examples above, the Nourin RNA molecular network was expressed in HF - high expression level of miR-137 and miR-106b and signaling pathways in the ISO / HF model was positively associated with: (a) CK-MB as indicative of myocardial injury, (b) echocardiographic LV ejection fraction, (c) cardiac fibrosis and remodeling, and (d) heart weight. Furthermore, since the level of Nourin biomarkers reflects the “severity” of myocardial injury and inflammation, it will accurately predict patients with high risk of developing HF after AMI. High level of Nourin is indicative of high probability of development of HF, while low level of Nourin is indicative of low probability of HF. Thus, Nourin is a new indicator of the degree of LV remodeling after AMI and and can be used as predictive of LV remodeling after AMI. Nourin will have the advantage over BNP of “independently” monitoring the progression of AMI patients for the development of HF without a need of all other clinical and physical assessment. The diagnostic strength of BNP is their high sensitivity for “ruling out” HF; as the value increases, HF becomes more likely. However, defining “rule-in” cutoffs for HF is complicated because multiple factors influence natriuretic peptide levels. Thus, Nourin RNA molecular network: (a) presents a new biomarker for left ventricular remodeling after myocardial infarction to “rule-in” HF patients, (b) prognostic value for “new-onset” HF, (c) risk prediction of progression and deterioration of cardiac function in patients with HF, (c) monitoring response to medical and surgical treatments to determine improvement or deterioration compared to before treatments (disease management), and (d) monitoring patients’ hearts in clinical trials to determine drug-induced cardiac toxicity (such in the case of Isoproterenol), as well as drugs that improve and prevent cardiac deterioration (such in the case of Cyclocreatine Phosphate).

CCrP treatment prevented ischemic injury and the development of HF in ISO rats by down regulation of gene expression of Nourin RNA network and cardiac inflammation. In several animal models of ischemia/reperfusion, there was, also, a significant reduction in the level of circulating Nourin protein, cardiac inflammation after CCrP treatment.

The Nourin family are tissue-derived inflammatory mediators rapidly released by various tissues in response to ischemic injury. Although tissue-derive Nourins share the same molecular weight of 3 KDa, they differ in their isoelectric points. The 3 KDa Nourin protein released by ischemic heart is designated as Nourin-1, while Nourin-2 is for ischemic brain, and Nourin-3 is for ischemic spinal cord. As indicated in this invention, the amino acid sequence of cardiac Nourin-1 and its genetic regulatory pathways, as well as clinical relevance have been determined. The amino acid sequences of brain Nourin-2 and spinal cord Nourin-3 have not determined yet.

Brain inflammation has been shown to play an important role in the development of reperfusion injury in brain ischemia and spinal cord and trauma. Since recruited neutrophils contribute to brain destruction in reperfusion injury, the release of Nourin-2 by brain tissues was investigated. Four pigs were sacrificed and brains were immediately removed, cut and incubated in Hank’s Balanced Salt Solution (HBSS) at room temperature (1 gm brain / 2 ml HBSS). After 5, 10, 20, 40, 60, and 240 minutes, 100 ul aliquots of supernatant solutions were collected and tested for the level of Nourin-2 using human peripheral neutrophils as indicator cells. Modified Boyden chambers were used to test for Nourin-2 neutrophil chemotactic activity in supernatant solutions (100 ul). The synthetic f-Met-Leu-Phe (f-MLF) (10⁻⁹ Molar) (SEQ ID NO:3) was used as positive control for 100% response. HBSS was used as negative control. Results were expressed as maximum chemotactic response of f-MLP. Results indicate significant release of Nourin-2 by ischemic brain tissues as early as 5 minutes (23-55% f-MLP) reaching maximum release by 40 minutes (77-91% f-MLP), then plateau for the remaining 4 hours (80-91% f-MLP). Samples collected from the 2-hour incubation were also processed using size exclusion high performance liquid chromatography (HPLC) using the 1-300 KDa fractionation column. High activity (57-102% f-MLP) was detected in fractions corresponded to fractions below 5 KDa. In conclusion, isolated ischemic pig brains rapidly produce a small molecular weight neutrophil chemotactic factor, Nourin-2, which in-vivo would not only promote inflammation but may also be useful as therapeutic target to reduce brain inflammation in stroke and trauma. Similarly, using pig spinal cord, the 3 KDa Nourin-3 was also rapidly released within 5 minutes of ischemia. Cyclocreatine which crosses blood brain barrier, protected pigs against ischemic injury and showed neuroprotective activity by restoring organ function (unpublished observation).

### Example 7- Clinical Applications of Cyclocreatine Phosphate as a Novel “Bioenergetic Therapy” to Prevent and Treat Ischemic as Well as Aging-Related Cardiovascular and Neurodegenerative Diseases

Heart and brain are among parts of the body requiring the greatest amounts of energy and they are the most affected during failures of the mitochondria to generate ATP due to ischemia and hypoperfusion. Mitochondrial dysfunction is relevance to ischemia, aging, ischemia-induced, and aging-related diseases and disorders such as cardiovascular, ocular ischemia-induced injury and diseases, optic nerve ischemia-induced injury and diseases, and retinal ischemia-induced injury and diseases, stroke, and Alzheimer’s disease. There is a link between the energy status of the cell and impaired organ function. Reduction of ATP production and the increase of oxidative stress are major triggers of neurons, and cardiac myocytes dysfunction, thereby contributing to the development and progression of age-related disorders. The progression of HF is associated with diminished energy metabolism and a decrease in ATP synthesis capacity and a decrease in overall ATP levels. Age-related changes in mitochondria are associated with decline in mitochondrial function and ATP production. Aging is characterized by a general decrease in O₂ supply to tissues and a reduction in tissue pO2. A diminished vascularization (lack of blood flow) in aging alters the diffusion of O2 at the capillary tissue level, and at an advanced stage, this can lead to tissue hypoxia. Preservation of ATP by CCrP treatment prevents ischemic injury, reduces disease progression and restores organ function. It will also slow down the aging process resulting in organ rejuvenation in of the aging-related diseases, HF, stroke, and Alzheimer’s disease, etc.

This example demonstrated that healthy rats treated with CCrP (0.8 gm/kg) for 14 days, showed no toxicity in heart, liver and renal function (Example 3). Since CCrP showed strong cardioprotective activities against ischemic heart diseases (AMI, bypass and HF), CCrP can also be useful to prevent and treat other cardiac ischemic diseases, including: atrial fibrillation. Takotsubo cardiomyopathy and cardiac surgeries, as well as neurodegenerative diseases, including: cerebral ischemic stroke and Alzheimer, and other neuro diseases such as ocular ischemia-induced injury and diseases, optic nerve ischemia-induced injury and diseases, and retinal ischemia-induced injury and diseases. Despite many therapies for patients with heart failure with reduced ejection fraction, such as angiotensin converting enzyme (ACE) inhibitors, angiotensin receptor blockers (ARBs), β blockers, and mineralocorticoid receptor antagonists, and advanced device therapies, hospital admissions for acute heart failure continue to increase and to date, no new therapies have improved clinical outcomes. Therefore, new drugs with “novel mechanisms of action”, such as CCrP which can improve contractile function in patients with reduced left ventricular ejection fraction may likely address these unmet needs for patients with heart failure.Additionally, although the survival rates for patients with heart failure have improved through current therapies (β-blockers, ACE inhibitors, angiotensin-II receptor blockers, and aldosterone antagonists) to relieve symptoms and reduced left ventricular remodeling and post-MI mortality, they did not result in prevention of the progression of disease. This could be related to complexity of the disease and the involvement of a number of underlying problems with structure or function of heart. Accordingly, heart transplantation is so far the only therapeutic option for end-stage heart failure.

Mitochondrial abnormalities and reduced capacity to generate ATP can have a profound impact in HF. Abnormal mitochondria are also linked to myocyte injury because they are a major source of reactive oxygen species (ROS) production that can induce cellular damage. Abnormal mitochondria also promote programmed cell death through the release of cytochrome c into the cytosolic compartment and activation of caspases. Bendavia was reported to improve cellular ATP levels and prevent pathological ROS formation. However, in the EMBRACE STEMI (Evaluation of Myocardial Effects of Bendavia for Reducing Reperfusion Injury in Patients With Acute Coronary Events - ST-Segment Elevation Myocardial Infarction) trial, elamipretide did not improve the primary or secondary outcomes. In the randomized placebo-controlled trial of elamipretide in HF, the drug was shown to reduce left ventricular volumes; however, the confidence intervals were wide in this small study, and there were no changes in biomarker data. Elamipretide is currently being investigated in larger HF studies to determine its effect on cardiac remodeling and clinical outcomes.

It was shown that depression of myocardial contractility plays an important role in the development of heart failure; therefore, there is a need for cardiotonic agents to improve the contractile function of the failing heart. Additionally, studies indicated that the development and progression to HF are associated with a decline in energy reserve capacity that ultimately reaches a threshold after which compensatory mechanisms can no longer support the decreasing energy supply. Growing evidence indicates that derangements in myocardial fuel metabolism and bioenergetics contribute to the development of heart failure. Stored myocardial high-energy phosphate (phosphocreatine) are reduced in humans with pathological ventricular hypertrophy, with further decline during the transition to heart failure. Notably, the [phosphocreatine]/[ATP] ratio correlates with heart failure severity and is a strong predictor of cardiovascular mortality. Thus, targeting energy metabolic disturbances and corresponding upstream regulatory events occurring during the early stages of HF is an important first step toward the identification of new therapeutic targets to improve the outcomes of current therapies. Mitochondrial energy source could, therefore, be a promising therapeutic target to improve mitochondrial biogenesis. Currently, there are no drugs that specifically target mitochondrial biogenesis in HF patients.

The immune system plays a significant role in ventricular remodeling, and its persistent activation may lead to long-term cardiac injury. Specifically, activation of a variety of inflammatory molecules and pathways, such as the complement system. T cells, and the formation of autoantibodies, have been reported in heart failure patients. Consequently, a number of strategies have been proposed to mitigate the harm caused by these inflammatory events; most have failed. In the 1970s, it became apparent that immunosuppression with glucocorticoids or nonsteroidal anti-inflammatory agents conferred risk in patients with ischemic heart disease. The degree of impaired contractile function after AMI is determined by the scar size: large scars result in progressive chronic heart failure. Furthermore, the influx of large number of neutrophils and inflammatory mediators after an AMI have been proposed as major contributors for microvascular obstruction and post-AMI adverse LV remodeling leading to heart failure.

Although inflammation is an important contributor to the pathogenesis of early and late myocardial reperfusion injury, and it also plays a key role in the “healing” process essential for cardiac repair and scar formation. Therefore, it is critical to achieve the right balance between limiting the early ‘harmful’ inflammation in the first few minutes to hours after reperfusion and allowing the ‘beneficial’ inflammation required for tissue repair. Since treating AMI patients with corticosteroids had a serious negative effect because they impaired and retarded wound healing, there is a need for new anti-inflammatory drugs that can control inflammation without affecting the healing process. It has been previously experimentally established that the adminstration of Cyclocreatine and Cyclocreatine Phosphate proved to be safe and effective with strong anti-inflammatory activity which protected ischemic hearts against reperfusion injury in 4 different animal models of ischemia/reperfusion: (1) AMI (2 hours reperfusion), (2) bypass surgery (4 hours), (3) heart transplantation (3 days), and the present disclosure provides it for (4) HF (14 days).

Since heart and brain are among parts of the body requiring the greatest amounts of energy and they are the most affected during failures of the mitochondria to generate ATP, preservation of the energy source ATP by CCrP will present a promising therapeutic approach as a new “age-modifier therapy” to prevent the development and to treat Alzheimer’s disease (AD) similar to HF (described in this invention). Due to the high energy demands of neurons and glia, a considerable amount of ATP is consumed in the brain. Also, because no energy storage (such as fat or glucose) is available in the central nervous system (CNS), brain cells must continually produce ATP to maintain activity and energy homeostasis . With aging, oxygen delivery to cells and tissues is impaired due to diminished vascularization, thereby increasing the susceptibility of neurons to damage. Thus, hypoxic (neuronal) adaptation is significantly compromised during aging . Many neurological diseases, such as stroke and Alzheimer’s disease (AD) are characterized by hypoxia, a state that is believed to only exacerbate disease progression. AD is a pressing public health problem with no effective treatment. Existing therapies only provide symptomatic relief without being able to prevent, stop or reverse the pathologic process. While the molecular basis underlying this multifactorial neurodegenerative disorder remains a significant challenge, mitochondrial dysfunction appears to be a critical factor in the pathogenesis of this disease. It is therefore important to target mitochondrial dysfunction in the prodromal phase of AD to slow or prevent the neurodegenerative process and restore neuronal function.

Studies reported mechanisms of action and translational potential of current mitochondrial and bioenergetic therapeutics for AD including: mitochondrial enhancers to potentiate energy production: antioxidants to scavenge reactive oxygen species and reduce oxidative damage; glucose metabolism and substrate supply; and candidates that target apoptotic and mitophagy pathways to remove damaged mitochondria. While mitochondrial therapeutic strategies have shown promise at the preclinical stage, there has been little progress in clinical trials thus far. Current FDA-approved drugs for AD treatment include: N-methyl-D-aspartic acid (NMDA) receptor antagonist memantine and cholinesterase inhibitors donepezil, galantamine, and rivastigmine. These drugs augment cholinergic neurotransmission or attenuate excitotoxic neuronal injury. However, they only provide palliative benefits at best, with limited impact on the underlying disease mechanisms. Therefore, there is an urgent need for interventions that not only impact the aging process in favor of sustained brain health but also promote successful brain aging in the context of neurodegenerative diseases.

The relationship between hypoxia and AD could open the avenue for effective preservation and pharmacological treatments of this neurodegenerative disease by using new therapeutic drugs like the novel bioenergetic drug, CCrP. CCrP provides protection of heart muscle against ischemic injury in CAD, UA, AMI, HF and cardiac surgical patients and thus save ischemic muscles from progressing to necrosis and heart failure, and will be protective against ischemic injury in stroke and AD. It has been previously demonstrated that Cyclocreatine crosses the blood brain barrier and functions as a potent neuroprotective agent by preventing ischemic injury and restoring organ function (unpublished observation). Since paraplegia following surgery of the descending thoracic aorta is a serious complication in adult and pediatric surgery, the neuroprotective effect of Cyclocreatine in Yorkshire pigs which underwent 30 minutes of aortic cross-clamping then left to survive for 4 days was tested. Majority of Cyclocreatine-treated animals were able to stand and walk, while, only few of the saline-treated control pigs were able to stand. This study suggests that Cyclocreatine administration prior to the induction of neural ischemia, protects against tissue injury and the development of paraplegia. As indicated in Example 4, similar to heart tissue (Nourin-1), Nourin protein was quickly released within 5 minutes by brain (Nourin-2) and spinal cord (Nourin-3) in response to ischemia. Because of the great similarities between heart and brain and that they are both require high demand of ATP, CCrP will be as effective in preventing ischemic injury and restoring neurologic function in stroke and AD, similar to HF.

The disclosure according to the invention provides a “novel mechanism of action” for tissue protection against ischemic injury using Cyclocreatine Phosphate to preserve cellular ATP energy source as a promising therapeutic approach to prevent the development and to treat HF patients. CCrP effective therapeutic approache, targeting preservation of ATP in ischemic myocardium, can mitigate the impact of inflammation and apoptosis and help restore post-ischemic cardiac function and normal physical activities.

As a novel mitochondria-targeted protective compound which prevents mitochondrial dysfunction, CCrP can be used for prevention and treatment not only cardiovascular, but also central nervous system diseases, including but not limited to Alzheimer and stroke. Since there is no energy storage (such as fat or glucose) is available in the central nervous system, brain cells must continually produce ATP to maintain activity and energy homeostasis. Additionally, since hypoxia is believed to continue to play a role in disease progression in HF, stroke and AD, continuing production of ATP by CCrP will be crucial for disease treatment by slowing or preventing disease progression and possibly reversing the pathologic process.

In summary, since hypoxia and reduction of ATP production are major triggers of cardiac myocytes and neurons dysfunction and they contribute to the “development” and “progression” of ischemia-induced and aging-related disorders, the below therapeutic strategies summarize a number of clinical protocols for CCrP adminstration to “prevent” and “treat” ischemia-induced and aging-related cardiovascular and neurodegenerative diseases, including:

1) HF - Since hypoxia and reduction of ATP production are major triggers and contributors not only in the “development” of HF, but also in disease “progression”, AMI patients will be treated IV with CCrP immediately after the ischemic event for 6 hours (IV), then orally daily for an additional 14 days to few weeks and months to prevent apoptosis and development of HF particularly for patients with large infarct scar size. CCrP can also be daily administered orally to patients with existing HF to provide the crucial cellular energy needed to prevent disease progression and thus, restore cardiac function and physical activity.

2) Stroke - CCrP can be orally administered prophylactically to high risk patients of brain stroke and aging population to protect against hypoxia/ischemic injury. CCrP can also be given as a “therapy” immediately after an ischemic event to protect brain tissue against deterioration of areas adjacent to ischemic tissues, thus, minimize cell injury and loss of brain function and disability.

3) Alzheimer - CCrP can be orally administered prophylactically to high risk patients of memory loss and aging population to protect against hypoxia/ischemic injury and, thus, reduces the loss of cognitive functions. CCrP can also be given as a therapy shortly after initiation of reduced cognitive, to prevent progression of tissue damage and loss of functions, thus minimizing severity of Alzheimer disease. By early providing the crucial ATP cellular energy, CCrP treatment may be able to prevent, stop or reverse the pathologic process of AD.

4) Aging - CCrP can function as anti-aging drug due to its ability to preserve mitochondrial function, thus will increase ATP production during the aging process. By decreasing apoptosis and inflammation, CCrP can preserve cognitive and motor functions. As an age-modifier therapy, CCrP can rejuvenation tissue by not only providing cellular energy (ATP), but also by maintaining healthy autophagy by inhibiting gene expression of Nourin-dependent m-R-137 (marker of ischemic injury) and miR-106b (marker of inflammation) with potential of reducing and slow down aging.

5) Based on the fact that CCrP prevented ischemic injury and the development of the ischemia-induced and aging-related heart failure disease, and since hypoxia/ischemia and reduction of ATP production are major triggers of neurons dysfunction and they contribute to the “development” and “progression” of ischemia-induced and and aging-related disorders such as Alzheimer’s disease, CCrP will potentially have therapeutic benefits in AD.

6) Cyclocreatine crosses the blood-brain barrier since in the blood. CCrP looses the phosphorous moiety and converts to Cyclocreatine.

7) It can be used in patients who will undergo nerve-related surgery, such as aneurysms, tumor, intracerebral hemorrhage surgery, vascular surgeries, ocular surgery, optic nerve surgery, retinal surgery, and other similar procedures (neuro muscular diseases), as well as patients who will undergo “non-nerve” related surgery that is capable of causing ischemia of the nervous system.

8) The present invention can be used during many treatment stages:
a) prophylactically prior to ischemia to protect against heart attack, stroke, peripheral nerve damage in high-risk patients including aging populations, diabetic patients, patients with vascular diseases including CAD, and patients with prior heart attack and transient ischemic attack (TIA).
b) immediately administered during ischemia to patients experiencing ischemic stroke at presentation to hospital ED, even beyond the three-hour therapeutic time window that often lead to treatment disqualification with thrombolytic therapy such as tPA. Unlike tPA therapy, CCrP would not be expected to have neurotoxic or vasoactive side effects, alter the blood brain barrier, or pose a risk of hemorrhage.

9) CCrP can be administered prophylactically, therapeutically during injury, or post-injury for continued therapy or prophylactically against recurrence.

10) CCrP can be administered by any suitable means, including, but not limited to injection, orally, topically, by inhalation, or by other means to prevent ischemic injury and treat ischemia-relating and aging diseases.

11) Thus, the present invention involves a system that provides for a three-stage treatment of (i) prevention, (ii) immediate therapy during ischemia, and (iii) post-ischemia rehabilitation to preserve, restore, and sustain organ function.

12) When administered shortly after incidence of ischemia it will:
a) preserve the “salvageable tissue” surrounding the ischemic and necrotic areas, by protecting injured tissues with the goal of preventing them from becoming irreversible damaged, thus, minimize devastating disability.
b) prevent and slow down disease progression.

### Example 8 - Expression of Nourin Protein in Response to Cardiac Ischemic Injury (Organ Culture)

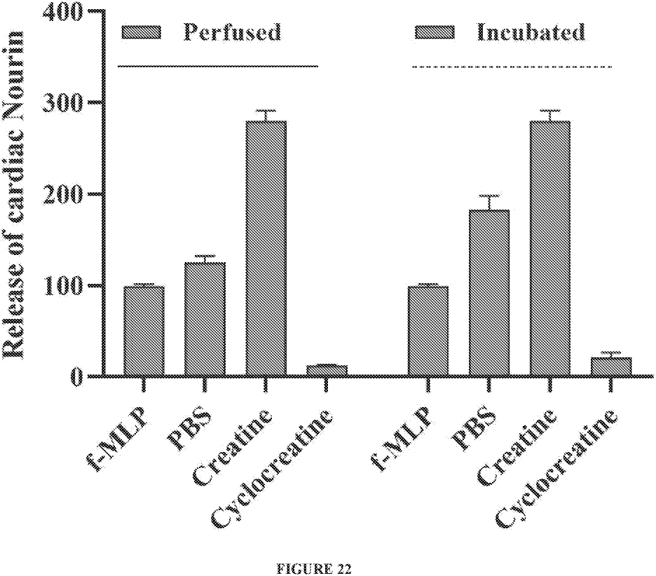

In this example, with the use of rabbit hearts, Cyclocreatine treatment was observed to inhibit the levels of cardiac Nourin released by said rabbit hearts (perfused and incubated) compared to non-treated hearts (creatine and buffer). FIG. 22 shows the high release of cardiac Nourin by isolated ischemic rabbit hearts. In this experiment, rabbits were injected intravenously 30 minutes before sacrifice with 5% cyclocreatine, 5% creatine, or saline, respectively. Hearts were then removed and perfused using the Langendorff technique or incubated in a beaker for 120 minutes with the above-described solutions. A minimum of four rabbit hearts were used for each variable. High neutrophil chemotactic activity (cardiac protein) recovered in perfusates and supernatants of ischemic hearts treated with creatine or PBS, while cyclocreatine-treated hearts showed marked reduction in detected Nourin protein. Interestingly, treating rabbits with creatine resulted in three-fold increase in Nourin protein as compared with control buffer, PBS.

This example demonstrates that using a heart organ culture model, there high elevated level of Nourin protein expression due to ischemic injury treated with creatine or buffer, which was remarkably reduced by the treatment with Cyclocreatine (CCr). The effects of CCr on high-energy phosphate measured in ischemic rabbit hearts (perfused) using high performance liquid chromatography (HPLC), revealed that ATP was quite low at isolated hearts treated with PBS buffer (0.10±0.01 umol/g wet weight) and creatine (0.18±0.03 umol/g wet weight). On the contrary, CCr treatment preserved high levels of ATP in these ischemic hearts (1.10±0.08 umol/g wet weight) with levels of 39% of healthy non-ischemic hearts (3.90±0.40 umol/g wet weight). Loss of 20% and more of ATP in ischemic hearts ceases cardiac function.

### Example 9 - Expression of Nourin Protein in Response to Vascular Ischemic Injury (Organ Culture)

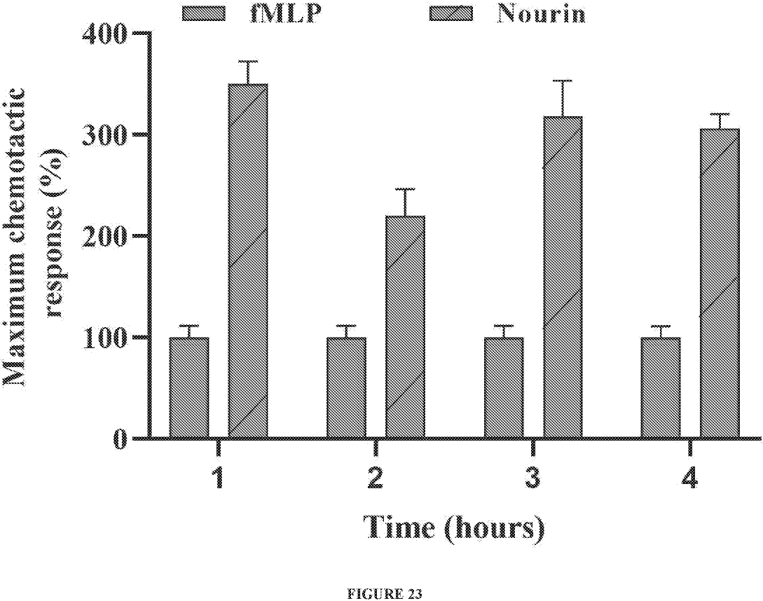

In this example as read with FIG. 23, it is shown that the release of Nourin protein occurred from isolated bovine coronary artery segments. The release of neutrophil chemotactic factor (Nourin protein) from vascular tissues was demonstrated using isolated bovine coronary arteries. Coronary arteries were isolated from fresh bovine hearts then cut into pieces and incubated for 1, 2, 3. and 4 hours at room temperature. High levels of chemotactic activity (2 to 3-fold of the positive control fMLF (SEQ ID NO: 1) was released by the 3 first hour of incubation and continued on for additional three hours (refer to Elgebaly et al., 1987).

Isolated Canine Vein Grafts: Significant release of neutrophil chemotactic factor was also detected in extended vein grafts of dogs. For these studies. leg veins were isolated and extended under pressure (300 mmHg) for 15 minutes then the inner surfaced allowed to incubate with buffer for an additional 45 minutes. High levels of chemotactic activity (5- to 7-fold) were detected in the inner buffer solutions isolated from the extended vein grafts compared to the in-situ control veins where the blood was removed and replaced with buffer for 1 hour without any extension (refer to Elgebaly et al., 1990).

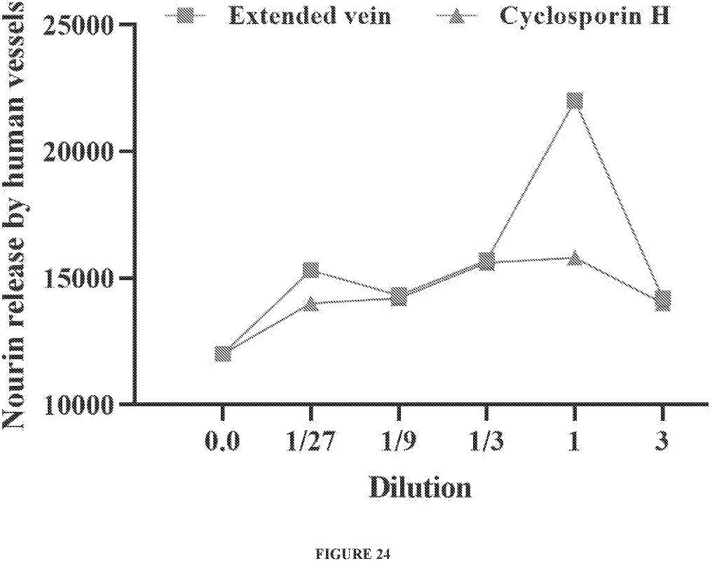

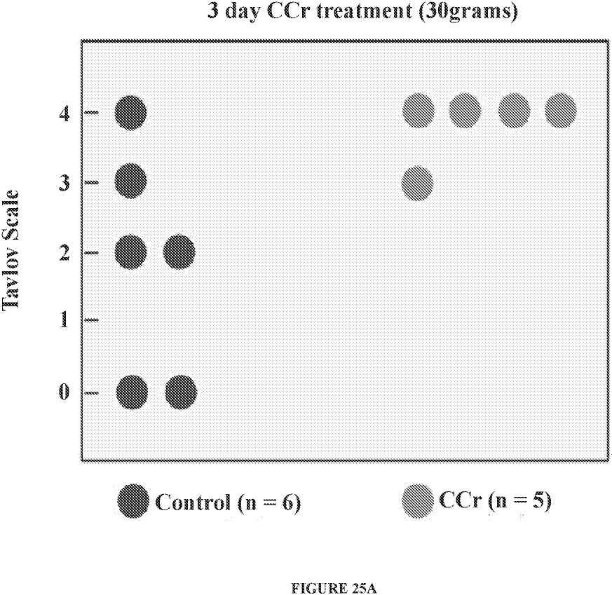

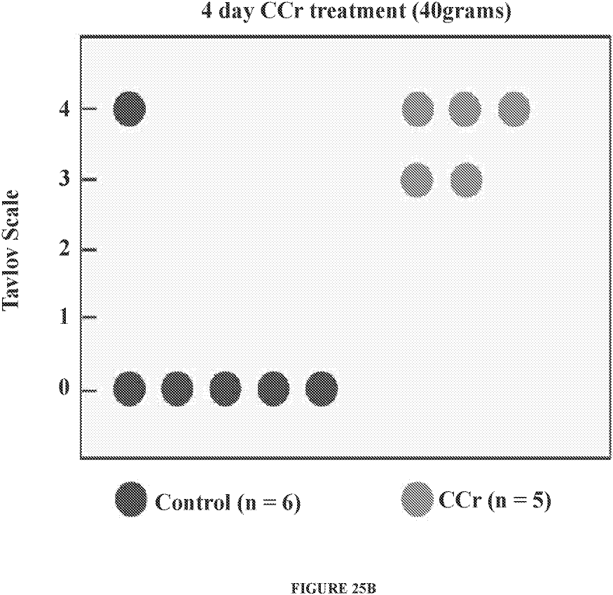

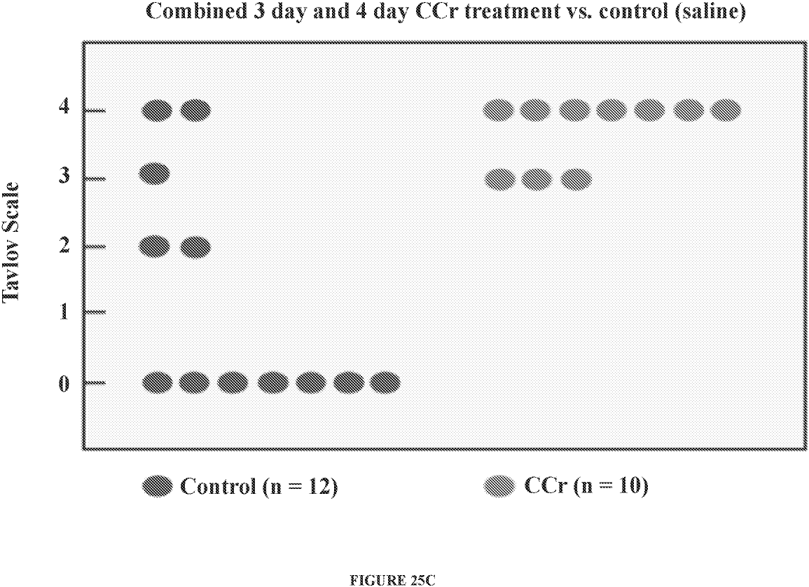

Additionally, FIG. 24 provides data for the release of Nourin protein from with isolated human vein grafts. Patients’ leg veins were removed from five patients scheduled for coronary bypass surgery. Veins from each patient were distended for 10 minutes at 300 mm Hg pressure and incubated in buffer (HBSS) for 1 hour at room temperature. Un-distended vein grafts were incubated at room temperature in HBSS for one hour and then the solutions were tested for chemotactic activity. Since Nourin can be characterized as an Alarmin and used as a crucial therapeutic target, Cyclosporin H was tested as a potent anti-inflammatory agent which competes with formyl peptides for the cellular formyl peptide receptor (FPR) on phagocytes. Cyclosporin H significantly inhibited in-vitro neutrophil chemotaxis induced by Nourin protein released in cell culture and in mice in response to influenza flu infection. Furthermore, in-vivo Nourexin-4 markedly reduced leukocyte infiltration to the lungs in the mouse model of influenza flu infection. Since Nourexin-4 is a specific competitive antagonist of Nourin on FPR, it will not affect other host defense immunity and will not subject patients to immunosuppression as seen in steroid treatments.

Additionally, the study reports an inhibition of the human vascular Nourin by the N-formyl methionyl blocker t-Boc-FLFLF (SEQ ID NO:4). t-Boc FLFLF, the amino acid sequence of FLFLF as set forth in SEQ ID NO:4 with a N-terminal phenylalanine is chemically modified to t-butoxycarbonyl-phenylalanine referred to as Boc-Phe, which is a FPR antagonist and was tested for its ability to inhibit Nourin activity on neutrophil migration. In the chemotaxis chamber, t-Boc-FLFLF (SEQ ID NO:4) was used at a final concentration of 10⁻⁵ Molar. Results indicated that that t-Boc-FLFLF (SEQ ID NO:4) at 10⁻⁵ M inhibited up to 60% of neutrophil chemotaxis induced by vascular Nourin. In one human sample, t-Boc-FLFLF (SEQ ID NO:4) inhibited 100% of neutrophil chemotaxis. Similarly, the FPR antagonist Spinorphin inhibited neutrophil chemotaxis stimulated by Nourin.

Results of this human study support that a potent chemotactic factor is rapidly released within 10 minutes by vascular tissues under non-physiological conditions including, ischemia and pressure extension. Vascular Nourin might, therefore be considered as an early potent mediator of vascular inflammation.

### Example 10 - Neuromuscular Protection by CCr Administration Against Ischemic Injury

Since the creatine analog Cyclocreatine (CCr) is a potent cardioprotective agent which maintains elevated levels of ATP during ischemia, its neuroprotection was evaluated in a pig model of ischemic-induced spinal cord injury. Paraplegia following surgery of the descending thoracic aorta is a serious complication in adult and pediatric surgery. This example shows that CCr is a neuromuscular protective agent against ischemia-induced spinal cord injury. Twenty-two Yorkshire swine underwent 30 minutes of aortic cross-clamping 1 centimeter distal to the left subclavian artery and at the level of the diaphragm.

CCr is water insoluble compound and it is soluble fully at 1% concentration. In this example, CCr-treated animals received daily intravenous infusion (i.e., one liter over 6-8 hours daily) of 1% CCr in saline solution (0.9% sodium chloride) for three days (total 30 gm, n=5), or four days (total 40 gm, n=5) prior to cross-clamping. Control animals received equal volume of saline for three (n=6) or four days (n=6). CCr function was evaluated 24 hours post-operatively using standard Tarlov Scale. As indicated in FIG. 25A to FIG. 25C. animals in both CCr groups were more likely to stand and walk than the controls (3 days results as shown in FIG. 25A: 100% (5/5) vs. 33% (2/6); 4 days results as shown in FIG. 25B: 100% (5/5) vs. 17% (⅙)). Combined, all ten CCr-treated animals were able to stand and walk, while three of the 12 controls were able to stand and walk (p=0.0001) results as shown in FIG. 25C. This study suggests that CCr administration prior to thoracic aortic cross-clamping protects against the development of paraplegia. In summary, administration of Cyclocreatine (CCr) shortly prior to the induction of ischemia via aortic cross-clamping (for example, four days or less prior to an ischemic condition) can be effective to restore post-ischemic nerve function. Such administration can have application to high-risk patients to protect against ischemic damage and reperfusion, as well as to presurgical patients to prevent loss of nerve function associated with post-ischemic reperfusion.

A follow up experiment using the sodium soluble salt, cyclocreatine phosphate (CCrP) - The major advantage of using CCrP instead of the insoluble CCr in the experiments as disclosed herein is to reduce the injection volume to patients scheduled for surgery of the descending thoracic aorta. However, unlike the heart, there is a need for the active ingredient. CCr/CCrP to accumulate in enough concentration in the neuro system to be effective. Accordingly, CCrP will be injected intravenously for 3 or 4 days, up to one week to patients prior to the date of the scheduled surgery of the descending thoracic aorta to receive the appropriate neuro protection.

### Example 11 - Expression of Nourin Protein in Response to Spinal Cord Ischemic Injury (Organ Culture)

In this example, three pigs were sacrificed and the spinal cord were immediately removed, cut (1 gm spinal cord/2 ml HBSS), and incubated in Hank’s Balance Salt Solution (HBSS) at room temperature. After 5, 10, 20,40,60, and 240 minutes. 100 micro-liter (µl) aliquots of supernatant solutions were collected and tested for the level of neutrophil chemotactic activity using human peripheral neutrophils as indicator cells. Samples collected from the 2-hour incubation were also processed on the size exclusion HPLC using the 1-300 KDa column. Modified Boyden chambers were used to test for the chemotactic activity in aliquot samples as undiluted, as well as diluted 1:5, and 1:25 in HBSS. The synthetic f-Met-Leu-Phe (SEQ ID NO:3) (10⁻⁹ Molar) (f-MLF) was used as the positive control for 100% response, while HBSS was used as the negative control for random migration . Neutrophil migration was reported as chemotactic index of cell density (O.D. Units) using a LKB laser densitometer.

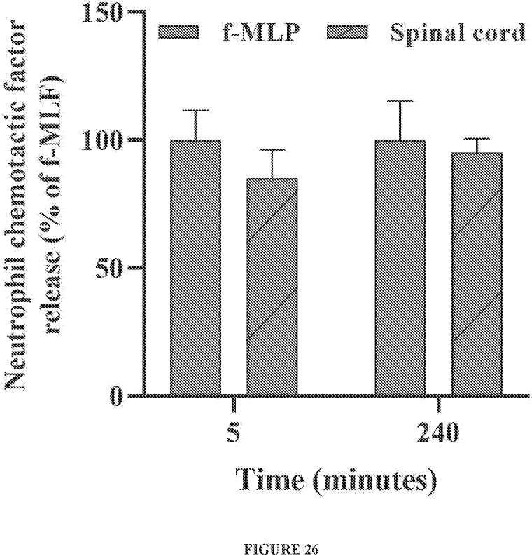

As seen with the data presented in FIG. 26, Nourin protein was released rapidly in response to ischemia injury in the spinal cord assessed as high levels of neutrophil chemotactic activity (75-95% of f-MLF (SEQ ID NO:3)), which was detected as early as 5 minutes post-ischemia. Activity continued to be detected (50 to 110 % of f-MLF (SEQ ID NO:3)) for the additional four-hour incubation. When culture supernatant solutions were fractionated using size exclusion HPLC (1-300 separation), high levels of chemotactic activity (50-100% f-MLF (SEQ ID NO:3)) was detected in fractions below 5 KDa. Activity was detected with undiluted, as well as fractions diluted as 1:5 and 1:25. This finding demonstrates the release of a potent low-molecular-weight chemotactic factor from ischemic spinal cord tissue minutes after ischemia and that the release of the factor was sustained for the duration of ischemia of 4 hours.

### Example 12 - Expression of Nourin Protein in Response to Brain Ischemic Injury (Organ Culture)

Brain inflammation has been shown to play an important role in the development of reperfusion injury in brain ischemia and trauma. Since recruited neutrophils contribute to brain destruction in reperfusion injury, in this example, the expression of neutrophil chemotactic factors (NCF) (Nourin protein) by brain tissues was investigated.

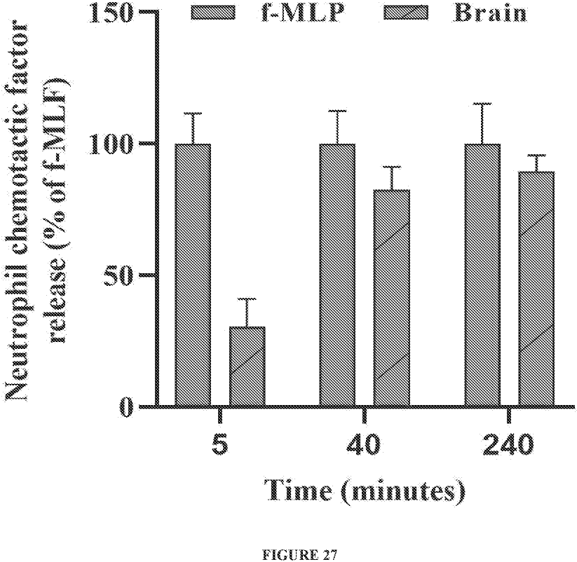

To begin determining the nature of NCF produced by brain tissues injured in-vitro, a simple brain organ culture was developed where the release of NCF from isolated pig brains was characterized. Four pigs were sacrificed and the brains were immediately removed, cut (1 gm brain / 2 ml HBSS) and incubated in Hank’s Balanced Salt Solution (HBSS) at room temperature . After 5, 10, 20, 40, 60, and 240 minutes, 100 µl aliquots of supernatant solutions were collected and tested for the level of neutrophil chemotactic activity using human peripheral neutrophils as indicator cells in the Boyden chambers. The synthetic f-Met-Leu- Phe (fMLF) was used as the positive control for 100% response. HBSS buffer was used as the negative control. Results were expressed as maximum chemotactic response of f-MLF (SEQ ID NO:3). As shown with the results in FIG. 27, a significant release of neutrophil chemotactic factors by ischemic brain tissues as early as 5 minutes (23-55% f-MLF) reaching maximum release by 40 minutes (77-91% f-MLF) was observed, which then plateaued for the remaining 4 hours (80-91% f-MLF). When culture supernatant solutions were collected after two hours of incubation and were then fractionated using size exclusion high performance liquid chromatography, high activity (57-102% f-MLF) was detected in fractions corresponded to fractions below 5 KDa.

In conclusion, isolated pig brains when injured in-vitro rapidly produce a small molecular weight neutrophil chemotactic factor, Nourin, which in-vivo would not only promote inflammation but may also be useful as an early marker of brain injury and inflammation. Furthermore, future characterization of this novel brain-derived neutrophil chemotactic factor and the development of blocking agents may likely provide an immuno-therapeutic approach to the treatment of stroke.

In summary, Nourin is released within minutes by ischemic brain. High and Low molecular weight Nourin Develop inhibitors against brain Nourin to protect against post-ischemic brain inflammation after stroke. Neuroprotection after stroke by cyclocreatine is demonstrated.

### Example 13 - Expression of Nourin Protein in Response to Retinal Ischemic and Oxidative Injury (Organ Culture)

In this example, isolated bovine choroids were incubated with buffer (ischemic injury) or with glucose (1 mg/ml) and glucose oxidase (20 U/ml) (oxidative injury) (Elgebaly SA et al., 1994). Chemotactic activity was measured using the standard modified Boyden chamber assay. F-MLF was used as 100% positive control and HBSS buffer as a negative control in the Boyden chamber. Using size exclusion HPLC, the low MW Nourin (below 5 KDa) in these samples was identified. Furthermore, pre-treatment of rabbits with dexamethasone (topically 1% t.i.d.) for 4 days did not inhibit the release of Nourin by isolated retinal tissue (Tyles E et al., 1994).

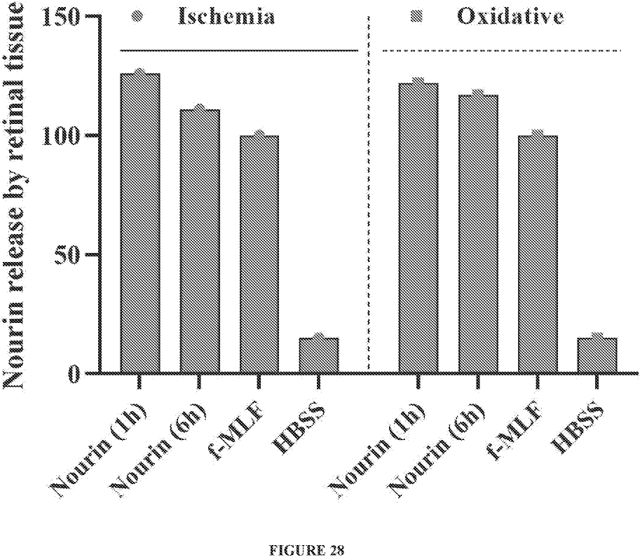

FIG. 28 shows high levels of the retinal chemotactic factor Nourin, which as shown therein is released after 1 hour and 6 hours in response to ischemic and oxidative injury in levels higher than the positive control f-MLP. Thus, Nourin is shown herein to be an effective target that can be targeted in a similar manner as disclosed herein to prevent and/or treat the effects of ischemic and oxidative injury in retinal system.

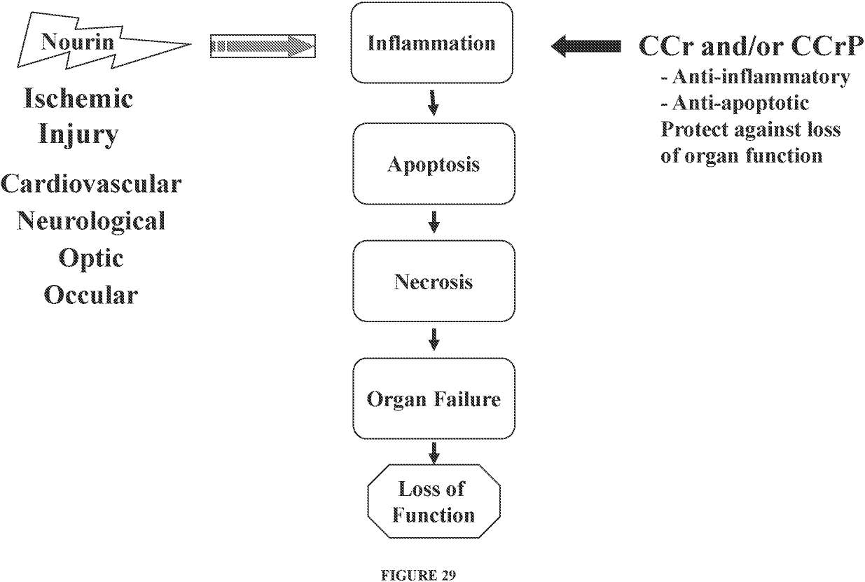

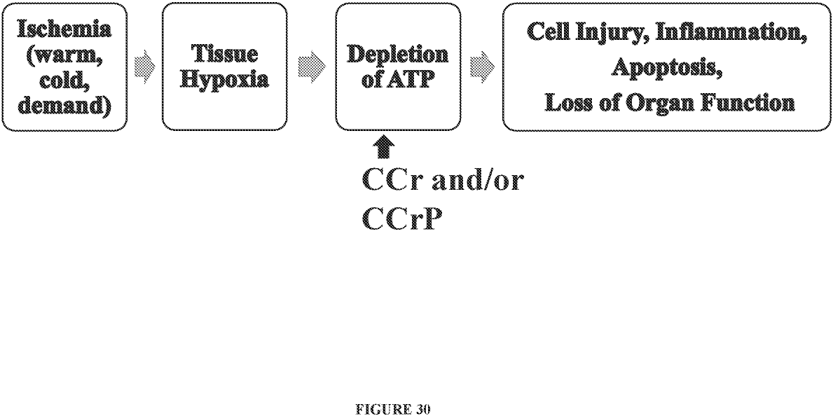

The above discussed examples considered together can be summarized in terms of FIG. 29 and FIG. 30 that provide the updated understanding and a new paradigm based on Nourin protein up-regulation in response to ischemic injury in high energy or ATP requiring organs particularly the heart or cardiac system and the brain or neuro system and in aging-related disorders involving ischemia injury and targeting the same with the use of cyclocreatinine (CCr) and/or cyclocreatine phosphate (CCrP) owing to its anti-inflammatory and anti-apoptotic functions key in ultimately protecting against loss of organ function (refer FIG. 29). Moreover, as established in cardiac systems with their cardioprotective roles. CCrP and CCr underlie a paradigm shift in the treatment of myocardial ischemia/hypoxia (refer FIG. 30). Bioenergetics prevents ischemic injury and endpoints of ischemia: inflammation, apoptosis and organ dysfunction and the same is protected against by CCrP and/or CCr as has been established for cardiac ischemia and its protection. The present disclosure establishes a similar new paradigm in neuro protection and treatment. While majority of existing treatment paradigm for ischemia-induced neuro and sometimes, aging-related neuro diseases such as Alzheimer’s disease targets the downstream accumulation of Aβ and tau proteins, the presently disclosed CCrP approach is a new paradigm that targets upstream ATP by enabling energy stores to be maintained even during reduction of blood reperfusion and tissue oxygenation, thus preventing ischemic injury and blocking the common pathways of cell injury, inflammation, apoptosis that can lead to protein accumulation and brain dysfunction as established herein.

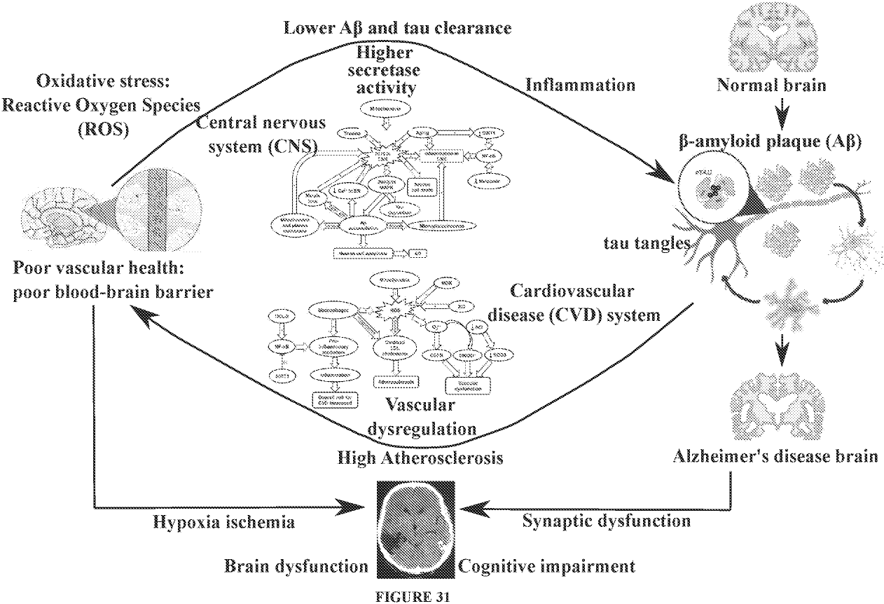

Finally, FIG. 31 summarizes and provides schematic representation showing the molecular mechanisms underlying (oxidative stress: reactive oxygen species (ROS))-, (inflammation)-, and (dysfunctional mitochondrial bioenergetics associated with ATP generation disruption)-mediated deregulation in neuro ischemic diseases and disorders involving ischemia-induced injury of central nervous system (CNS), including aging-related disorders as disclosed in the present disclosure based on the paradigm developed and presently disclosed in the cardiovascular disease (CVD) system. In the CNS, in neuro ischemic diseases and disorders involving ischemia-induced injury, including aging-related disorders, such as Alzheimer’s disease, where for instance, pathogenesis of Alzheimer’s disease is linked with mitochondrial dysfunction and reduction of cellular ATP, as well as intracellular deposition of tau neurofibrillary tangles or tau tangles and extracellular accumulation of β-amyloid plaques (Aβ) that transform a normal brain into Alzheimer’s disease brain, characterized by lower Aβ and tau clearance, higher secretase activity, as well as vascular dysregulation and high intracranial atherosclerosis also referred to as stenosis resulting in poor vascular health owing to poor blood-brain barrier in Alzheimer’s disease brain, which respectively lead to synaptic dysfunction and hypoxia ischemia ending in brain dysfunction and cognitive impairment. The present disclosure targets the aforementioned molecular mechanisms via the disclosed bioenergetic drugs, cyclocreatine (CCr) and/or cyclocreatine phosphate (CCrP) to counter the depletion of ATP in such organs to combat and prevent consequent cell injury, inflammation, apoptosis, and ultimate loss of organ function.

It is the Nourin target therapy as provided in the present disclosure that works similarly for cardiovascular diseases as it does for neuro system diseases including but not limited to Alzheimer’s disease, hypoxia/ischemia-induced stroke, aging-related stroke, ocular ischemia-induced injury and diseases, optic nerve ischemia-induced injury and diseases, and retinal ischemia-induced injury and diseases, as well as neurological surgical procedures. As disclosed herein, Nourin targeting preserves mitochondrial biogenesis, prevents cell injury, prevents disease development, rejuvenates organ function, restores normal physical activity driven by CCr/CCrP’s anti-inflammatory and anti-apoptotic activity, which is responsible for prevention of ischemic injury and for restoring organ function without affecting the host defense immunity, which in turn does not subject the patients to immunosuppression as seen with steroid treatments given to such patients. The present disclosure takes advantage of and is a continuation of the Nourin targeting based treatment paradigm first established in the cardiovascular system (heart and vessels) via CCr/CCrP targeting inflammation and apoptosis as shown in FIG. 1 providing the current protocols of cardiovascular protection, which is extrapolated in the present disclosure to the neuro system (brain, optic, ocular, spinal cord, and retina system), which is similar in its requirements of the greatest amounts of energy and for being the most affected during failures of the mitochondria to generate sufficient cellular adenosine triphosphate (ATP) during hypoxia and ischemia as well as in an aging-related manner. The underlying reasoning stems from the fact that both the cardiovascular system and neuro systems as describe hereinabove are sensitive to changes in oxygen supply and ischemia and inflammation play a key role in the said organ systems. The bioenergetic drugs, CCr and CCrP of the present disclosure work by impacting ATP depletion, oxidative stress, and inflammation in ischemic cardiac and neuro systems, which in turn are critical contributors to the pathogenic events that triggers cell injury, inflammation/fibrosis, apoptosis, leading to organ dysfunction, and ultimately organ function loss in patients as summarized in FIG. 29 and FIG. 31.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed:

1. A method for preventative and therapeutic treatment of neuro ischemic diseases and disorders involving ischemia-induced injury, the method comprising the steps of:
(a) recruiting a subject;
(b) monitoring the subject for presence of ischemic events, ischemia-induced injury, and tissue deterioration by assessing the levels of external markers for the ischemic events, ischemia-induced injury, and tissue deterioration known for characterizing neuro ischemic diseases and disorders involving ischemia-induced injury;
(c) collecting a first set of samples from the subject:
(d) analyzing the first set of samples and assessing for the expression and release of neutrophil chemotactic factor referred to as Nourin protein levels, for ATP levels, and for the levels of internal markers of the downstream determiners for the ischemic events, ischemia-induced injury, and tissue deterioration selected from inflammation, apoptosis, necrosis, organ failure, and loss of organ function markers known for characterizing neuro ischemic diseases and disorders involving ischemia-induced injury;
(e) classifying the monitored subject in terms of the levels of said external markers of step (b), and the levels of said internal markers of step (d) to determine the stage and progress of the ischemic events, ischemia-induced injury, and tissue deterioration in the subject to understand the severity of the neuro ischemic diseases and disorders involving ischemia-induced injury and to calculate a therapeutically effective amount of a bioenergetic agent to be administered to the subject;
(f) administrating to the subject the therapeutically effective amount of the bioenergetic agent as calculated in step (e);
(g) monitoring the subject again at various time-intervals after said administrating of the bioenergetic agent by assessing the levels of external markers for the ischemic events, ischemia-induced injury, and tissue deterioration known for characterizing neuro ischemic diseases and disorders involving ischemia-induced injury;
(h) collecting from the subject a second set of samples after administrating the bioenergetic agent and subsequent sets of samples at various time-intervals after said administrating;
(i) analyzing the second set of samples and the subsequent sets of samples of step (h) and assessing for the expression and release of neutrophil chemotactic factor referred to as Nourin protein levels, for ATP levels, and for the levels of internal markers of the downstream determiners for the ischemic events, ischemia-induced injury, and tissue deterioration selected from inflammation, apoptosis, necrosis, organ failure, and loss of organ function markers known for characterizing neuro ischemic diseases and disorders involving ischemia-induced injury;
(j) calculating the effectiveness of the bioenergetic agent in terms of the expression and release of neutrophil chemotactic factor referred to as Nourin protein, and the ATP levels by comparing their levels in the first set of samples as analyzed in step (d) to the second set of samples and subsequent sets of samples after said administrating as analyzed in step (i);
(k) calculating the presence, progress, and stage of the ischemic events, ischemia-induced injury, and tissue deterioration after said administrating of the bioenergetic agent in terms of the levels of external markers for the ischemic events, ischemia-induced injury, and tissue deterioration as assessed in step (b) in comparison to as assessed after said administrating in step (g), and the levels of internal markers of the downstream determiners for the ischemic events, ischemia-induced injury, and tissue deterioration selected from inflammation, apoptosis, necrosis, organ failure, and loss of organ function markers by comparing their levels in the first set of samples as analyzed in step (d) to the second set of samples and subsequent sets of samples after said administrating as analyzed in step (i) to check the effectivity of the bioenergetic agent in halting or reversing progress, prevention, or treatment of neuro ischemic diseases and disorders involving ischemia-induced injury,
wherein the bioenergetic agent is a synthetic analogue that maintains and restores mitochondrial bioenergetics associated with ATP generation disrupted in ischemic events and ischemia-induced injury associated with neuro ischemic diseases and disorders involving ischemia-induced injury,
wherein the ischemia-induced injury comprises a tissue in an organ of the subject exposed to injury, hypoxia, or ischemia associated with neuro ischemic diseases and disorders involving ischemia-induced injury, and wherein the ischemia comprises warm, cold, or demand ischemia, wherein the bioenergetic agent preserves mitochondrial biogenesis, prevents cell injury, prevents disease development, rejuvenates organ function, and restores normal physical activity, and
wherein the bioenergetic agent is selected from 1-carboxymethyl-2-iminoimidazolidine referred to as Cyclocreatine (CCr), and 1-carboxymethyl-2-imino-3-phosphonoimidazolidine referred to as Cyclocreatine phosphate (CCrP).

2. The method of claim 1, wherein the therapeutically effective amount of a bioenergetic agent given to the subject comprises administrating an amount of the bioenergetic agent in a range between 0.3 g per kg per day and 1.5 g per kg per day of body weight as calculated for administrating the bioenergetic agent once daily or more than once daily for a cumulative amount administered to be in a range of between 0.3 g per kg per day and 1.5 g per kg per day of body weight based on step (e) of claim 1.

3. The method of claim 1, wherein the bioenergetic agent is administered daily at an amount as calculated in step (e) of claim 1 such that there is no change in heart, liver, brain, and kidney function which is indicative of absence of organ toxicity.

4. The method of claim 1, wherein the administrating of the bioenergetic agent is selected from a group consisting of:
prophylactic administration of the bioenergetic agent by injection daily, in a range of time between 7 days and 10 minutes prior to, or by injection immediately prior to injury or in a range of time between a few hours and 30 days post injury to protect against ischemic damage, and to prevent, treat, inhibit, or reduce tissue deterioration and disease progression associated with the downstream determiners selected from inflammation, apoptosis, necrosis, organ failure, and loss of organ function, for prevention of neuro ischemic diseases and disorders involving ischemia-induced injury, and
therapeutic administration of the bioenergetic agent during injury or immediately post-injury for few hours, then daily for 1, 7 to 14 days, weeks, months and years to treat ischemic disease, to prevent and slowdown tissue deterioration, to inhibit or reduce ischemic disease progression associated with the downstream determiners selected from inflammation, apoptosis, necrosis, organ failure, and loss of organ function, and to stop or reverse the pathologic process for treatment of neuro ischemic diseases and disorders involving ischemia-induced injury.

5. The method of claim 1, wherein the bioenergetic agent 1-carboxymethyl-2-iminoimidazolidine referred to as Cyclocreatine (CCr) is insoluble form of the bioenergetic agent, and 1-carboxymethyl-2-imino-3-phosphonoimidazolidine referred to as Cyclocreatine phosphate (CCrP) is soluble form of the bioenergetic agent, and wherein the bioenergetic agent is administered in a form, in a solvent, and in solution so as to cross the blood-brain barrier to be effective in prevention and treatment of neuro ischemic diseases and disorders involving ischemia-induced injury.

6. The method of claim 1, wherein the bioenergetic agent is administered by a route of administration selected from a group of routes of administration comprising, intravenously, intraperitoneally, orally, intranasally, subcutaneously, intrathecally, intraventricularly, and intramuscularly.

7. The method of claim 1, wherein the subject is a human being or an animal, wherein the subject is a healthy subject or a diseased subject, and wherein the diseased subject is characterized and classified as suffering from neuro ischemic diseases and disorders involving ischemia-induced injury in terms of the levels of external markers for the ischemic events, ischemia-induced injury, and tissue deterioration, and the levels of internal markers of the downstream determiners for the ischemic events, ischemia-induced injury, and tissue deterioration selected from inflammation, apoptosis, necrosis, organ failure, and loss of organ function markers as compared to known standard levels of said external markers and said internal markers in healthy subjects.

8. The method of claim 1, wherein the external markers for the ischemic events, ischemia-induced injury, and tissue deterioration known for characterizing neuro ischemic diseases and disorders involving ischemia-induced injury comprise motor and sensory skills, balance and coordination, mental status which assesses the subject’s level of awareness and interaction with the environment, reflexes, cognitive screening tests, and functioning of the nerves, and wherein said external markers are assessed depending on factors, including the initial problem that the subject is experiencing or presenting with, the age of the subject, and the condition of the subject.

9. The method of claim 1, wherein the internal markers of the downstream determiners for the ischemic events, ischemia-induced injury, and tissue deterioration selected from inflammation, apoptosis, necrosis, organ failure, and loss of organ function markers known for characterizing neuro ischemic diseases and disorders involving ischemia-induced injury comprise inflammatory and anti-inflammatory markers comprising cytokine and chemokine levels, apoptosis and anti-apoptosis markers, necrosis and anti-necrosis markers, markers for organ failure of organs including brain, liver, heart, kidney, vasculature, and markers for loss of organ function of organs including brain, liver, heart, kidney, vasculature.

10. The method of claim 1, wherein the neuro ischemic diseases and disorders involving ischemia-induced injury comprise a group selected from Alzheimer’s disease, hypoxia/ischemia-induced stroke, ocular ischemia-induced injury and diseases, optic nerve ischemia-induced injury and diseases, and retinal ischemia-induced injury and diseases.

11. A method for prevention and treatment of ischemic events and ischemia-induced injury and tissue deterioration associated with neurologic surgical procedures, the method comprising the steps of:
(i) recruiting a subject set to undergo a neurologic surgical procedure:
(ii) monitoring the subject for presence of ischemic events, ischemia-induced injury and tissue deterioration by assessing the levels of external markers for the presence of ischemic events, ischemia-induced injury and tissue deterioration known to be associated with neurologic surgical procedures;
(iii) collecting a first set of samples from the subject before the performance of the neurologic surgical procedure that the subject is set to undergo;
(iv) analyzing the first set of samples and assessing for the expression and release of neutrophil chemotactic factor referred to as Nourin protein levels, for ATP levels, and for the levels of internal markers of the downstream determiners for the ischemic events, ischemia-induced injury and tissue deterioration selected from inflammation, apoptosis, necrosis, organ failure, and loss of organ function markers known to be associated with neurologic surgical procedures;
(v) classifying the monitored subject in terms of the levels of said external markers of step (ii), and the levels of said internal markers of step (iv) to determine the stage and progress of the ischemic events, ischemia-induced injury and tissue deterioration in the subject to understand the condition of the subject set to undergo the neurologic surgical procedure and to calculate a therapeutically effective amount of a bioenergetic agent to be administered to the subject:
(vi) administrating to the subject the therapeutically effective amount of the bioenergetic agent as calculated in step (v);
(vii) monitoring the subject again at various time-intervals after said administrating of the bioenergetic agent by assessing the levels of external markers for the ischemic events, ischemia-induced injury and tissue deterioration known to be associated with neurologic surgical procedures;
(viii) collecting from the subject a second set of samples after administrating the bioenergetic agent and subsequent sets of samples at various time-intervals after said administrating;
(ix) analyzing the second set of samples and the subsequent sets of samples of step (viii) and assessing for the expression and release of neutrophil chemotactic factor referred to as Nourin protein levels, for ATP levels, and for the levels of internal markers of the downstream determiners for the ischemic events, ischemia-induced injury and tissue deterioration selected from inflammation, apoptosis, necrosis, organ failure, and loss of organ function markers known to be associated with neurologic surgical procedures;
(x) calculating the effectiveness of the bioenergetic agent in terms of the expression and release of neutrophil chemotactic factor referred to as Nourin protein, and the ATP levels by comparing their levels in the first set of samples as analyzed in step (iv) to the second set of samples and subsequent sets of samples after said administrating as analyzed in step (ix);
(xi) calculating the presence, progress, and stage of the ischemic events, ischemia-induced injury and tissue deterioration after said administrating of the bioenergetic agent in terms of the levels of external markers as assessed in step (ii) in comparison to as assessed after said administrating in step (vii), and the levels of internal markers as analyzed in step (iv) in comparison to the as assessed after said administrating as analyzed in step (ix) to check the effectivity of the bioenergetic agent in halting or reversing progress, prevention, or treatment of ischemic events, ischemia-induced injury and tissue deterioration associated with neurologic surgical procedures,
wherein the bioenergetic agent is a synthetic analogue that maintains and restores mitochondrial bioenergetics associated with ATP generation disrupted in ischemic events, ischemia-induced injury and tissue deterioration associated with neurologic surgical procedures,
wherein the ischemia-induced injury comprises a tissue in an organ of the subject exposed to injury, hypoxia, or ischemia associated with neurologic surgical procedures involving ischemia-induced injury, and wherein the ischemia comprises warm, cold, or demand ischemia,
wherein the bioenergetic agent preserves mitochondrial biogenesis, prevents cell injury, prevents disease development, rejuvenates organ function, and restores normal physical activity, and
wherein the bioenergetic agent is selected from 1-carboxymethyl-2-iminoimidazolidine referred to as Cyclocreatine (CCr), and 1-carboxymethyl-2-imino-3-phosphonoimidazolidine referred to as Cyclocreatine phosphate (CCrP).

12. The method of claim 11, wherein the therapeutically effective amount of a bioenergetic agent given to the subject comprises administrating an amount of the bioenergetic agent in a range between 0.3 g per kg per day and 1.5 g per kg per day of body weight as calculated for administrating the bioenergetic agent once daily or more than once daily for a cumulative amount administered to be in a range of between 0.3 g per kg per day and 1.5 g per kg per day of body weight based on step (v) of claim 11.

13. The method of claim 11, wherein the bioenergetic agent is administered daily at an amount as calculated in step (v) of claim 11 such that there is no change in heart, liver, brain, and kidney function which is indicative of absence of organ toxicity.

14. The method of claim 11, wherein the administrating of the bioenergetic agent is selected from a group consisting of:
prophylactic administration of the bioenergetic agent by injection daily, in a range of time between 7 days and 10 minutes prior to, or by injection immediately prior to when the subject is set to undergo a neurologic surgical procedure or in a range of time between a few hours and 30 days post the neurological procedure to protect against ischemic damage, and to prevent, treat, inhibit, or reduce tissue deterioration and disease progression associated with the downstream determiners selected from inflammation, apoptosis, necrosis, organ failure, and loss of organ function, for prevention of ischemic events, ischemia-induced injury and tissue deterioration known to be associated with neurologic surgical procedures, and
therapeutic administration of the bioenergetic agent during the neurological procedure or immediately post the neurological procedure for few hours, then daily for 1, 7 to 14 days, weeks, months and years to treat ischemic disease, to prevent and slowdown tissue deterioration, to inhibit or reduce ischemic disease progression associated with the downstream determiners selected from inflammation, apoptosis, necrosis, organ failure, and loss of organ function, and to stop or reverse the pathologic process for treatment of ischemic events, ischemia-induced injury and tissue deterioration associated with neurologic surgical procedures.

15. The method of claim 11, wherein the bioenergetic agent 1-carboxymethyl-2-iminoimidazolidine referred to as Cyclocreatine (CCr) is insoluble form of the bioenergetic agent, and 1-carboxymethyl-2-imino-3-phosphonoimidazolidine referred to as Cyclocreatine phosphate (CCrP) is soluble form of the bioenergetic agent, and wherein the bioenergetic agent is administered in a form, in a solvent, and in solution so as to cross the blood-brain barrier to be effective in prevention and treatment of ischemic events and ischemia-induced injury and tissue deterioration associated with neurologic surgical procedures.

16. The method of claim 11, wherein the bioenergetic agent is administered by a route of administration selected from a group of routes of administration comprising, intravenously, intraperitoneally, orally, intranasally, subcutaneously, intrathecally, intraventricularly, and intramuscularly.

17. The method of claim 11, wherein the subject is a human being or an animal.

18. The method of claim 11, wherein the external markers for the presence of ischemic events, ischemia-induced injury and tissue deterioration known to be associated with neurologic surgical procedures comprise motor and sensory skills, balance and coordination, mental status which assesses the subject’s level of awareness and interaction with the environment, reflexes, cognitive screening tests, and functioning of the nerves, and wherein said external markers are assessed depending on factors, including the initial problem that the subject is experiencing or presenting with, the age of the subject, and the condition of the subject.

19. The method of claim 11, wherein the internal markers of the downstream determiners for the presence of ischemic events, ischemia-induced injury and tissue deterioration selected from inflammation, apoptosis, necrosis, organ failure, and loss of organ function markers known to be associated with neurologic surgical procedures comprise inflammatory and anti-inflammatory markers comprising cytokine and chemokine levels, apoptosis and anti-apoptosis markers, necrosis and anti-necrosis markers, markers for organ failure of organs including brain, liver, heart, kidney, vasculature, and markers for loss of organ function of organs including brain, liver, heart, kidney, vasculature.

20. The method of claim 11, wherein the neurologic surgical procedures comprise intracerebral hemorrhage surgery, arterial repair, ocular surgery, optic nerve surgery, retinal surgery, non-nerve-related surgery that is capable of causing ischemia of the nervous system and other brain surgery to protect against ischemic damage, and to prevent, inhibit or reduce tissue deterioration and disease progression.
